SEQUENCE LISTING (576 sequences)

SEQ ID NO: 1 - protein, 26 aa, synthetic construct
  REGION 1..26: note = H-FR1 Peptide
QVQLVQSGAEVKKPGSSVKVSCKASG

SEQ ID NO: 2 - protein, 14 aa, synthetic construct
  REGION 1..14: note = H-FR2 Peptide
WVRQAPGQGLEWMG

SEQ ID NO: 3 - protein, 30 aa, synthetic construct
  REGION 1..30: note = H-FR3 Peptide
RVTITADESTSTAYMELSSLRSEDTAVYYC

SEQ ID NO: 4 - protein, 11 aa, synthetic construct
  REGION 1..11: note = H-FR4 Peptide
WGQGTMVTVSS

SEQ ID NO: 5 - DNA, 78 bp, synthetic construct
  misc_feature 1..78: note = H-FR1 DNA
  source 1..78: mol_type = other DNA
caggtgcagctggtgcagtctggggctgaggtgaagaagcctgggtcctcggtgaaggtctcctgcaaggcttctgga

SEQ ID NO: 6 - DNA, 42 bp, synthetic construct
  misc_feature 1..42: note = H-FR2 DNA
  source 1..42: mol_type = other DNA
tgggtgcgacaggcccctggacaagggcttgagtggatggga

SEQ ID NO: 7 - DNA, 90 bp, synthetic construct
  misc_feature 1..90: note = H-FR3 DNA
  source 1..90: mol_type = other DNA
agagtcacgattaccgcggacgaatccacgagcacagcctacatggagctgagcagcctgagatctgaggacacggcggtgtactactgc

SEQ ID NO: 8 - DNA, 33 bp, synthetic construct
  misc_feature 1..33: note = H-FR4 DNA
  source 1..33: mol_type = other DNA
tggggtcagggtacaatggtcaccgtctcctca

SEQ ID NO: 9 - protein, 9 aa, synthetic construct
  REGION 1..9: note = HCDR1 Peptide
GTFSNYAIS

SEQ ID NO: 10 - protein, 17 aa, synthetic construct
  REGION 1..17: note = HCDR2 Peptide
GIIPIFGTANYAQKFQG

SEQ ID NO: 11 - protein, 16 aa, synthetic construct
  REGION 1..16: note = HCDR3 Peptide
AREARVAGTRPRGFDI

SEQ ID NO: 12 - DNA, 27 bp, synthetic construct
  misc_feature 1..27: note = HCDR1 DNA
  source 1..27: mol_type = other DNA
ggcaccttcagcaactatgctatcagc

SEQ ID NO: 13 - DNA, 51 bp, synthetic construct
  misc_feature 1..51: note = HCDR2 DNA
  source 1..51: mol_type = other DNA
gggatcatccctatctttggtacagcaaactacgcacagaagttccagggc

SEQ ID NO: 14 - DNA, 48 bp, synthetic construct
  misc_feature 1..48: note = HCDR3 DNA
  source 1..48: mol_type = other DNA
gccagagaggctagagtagcaggaacacgaccacgtggattcgacata

SEQ ID NO: 15 - protein, 123 aa, synthetic construct
  REGION 1..123: note = VH Peptide
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCAREARVAGTRPRGFDIWGQGTMVTVSS

SEQ ID NO: 16 - DNA, 369 bp, synthetic construct
  misc_feature 1..369: note = VH DNA
  source 1..369: mol_type = other DNA
caggtgcagctggtgcagtctggggctgaggtgaagaagcctgggtcctcggtgaaggtctcctgcaaggcttctggaggcaccttcagcaactatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagagaggctagagtagcaggaacacgaccacgtggattcgacatatggggtcagggtacaatggtcaccgtctcctca

SEQ ID NO: 17 - protein, 23 aa, synthetic construct
  REGION 1..23: note = L-FR1 Peptide
EIVLTQSPATLSLSPGERATLSC

SEQ ID NO: 18 - protein, 15 aa, synthetic construct
  REGION 1..15: note = L-FR2 Peptide
WYQQKPGQAPRLLIY

SEQ ID NO: 19 - protein, 32 aa, synthetic construct
  REGION 1..32: note = L-FR3 Peptide
GIPARFSGSGSGTDFTLTISSLEPEDFAVYYC

SEQ ID NO: 20 - protein, 10 aa, synthetic construct
  REGION 1..10: note = L-FR4 Peptide
FGGGTKVEIK

SEQ ID NO: 21 - DNA, 69 bp, synthetic construct
  misc_feature 1..69: note = L-FR1 DNA
  source 1..69: mol_type = other DNA
gaaattgtgttgacacagtctccagccaccctgtctttgtctccaggggaaagagccaccctctcctgc

SEQ ID NO: 22 - DNA, 45 bp, synthetic construct
  misc_feature 1..45: note = L-FR2 DNA
  source 1..45: mol_type = other DNA
tggtaccaacagaaacctggccaggctcccaggctcctcatctat

SEQ ID NO: 23 - DNA, 96 bp, synthetic construct
  misc_feature 1..96: note = L-FR3 DNA
  source 1..96: mol_type = other DNA
ggcatcccagccaggttcagtggcagtgggtctgggacagacttcactctcaccatcagcagcctagagcctgaagattttgcagtttattactgt

SEQ ID NO: 24 - DNA, 30 bp, synthetic construct
  misc_feature 1..30: note = L-FR4 DNA
  source 1..30: mol_type = other DNA
tttggcggagggaccaaggttgagatcaaa

SEQ ID NO: 25 - protein, 11 aa, synthetic construct
  REGION 1..11: note = LCDR1 Peptide
RASQSVSSYLA

SEQ ID NO: 26 - protein, 7 aa, synthetic construct
  REGION 1..7: note = LCDR2 Peptide
DASNRAT

SEQ ID NO: 27 - protein, 9 aa, synthetic construct
  REGION 1..9: note = LCDR3 Peptide
QQLNNFPPT

SEQ ID NO: 28 - DNA, 33 bp, synthetic construct
  misc_feature 1..33: note = LCDR1 DNA
  source 1..33: mol_type = other DNA
agggccagtcagagtgttagcagctacttagcc

SEQ ID NO: 29 - DNA, 21 bp, synthetic construct
  misc_feature 1..21: note = LCDR2 DNA
  source 1..21: mol_type = other DNA
gatgcatccaacagggccact

SEQ ID NO: 30 - DNA, 27 bp, synthetic construct
  misc_feature 1..27: note = LCDR3 DNA
  source 1..27: mol_type = other DNA
cagcagctcaacaatttccctcctact

SEQ ID NO: 31 - protein, 107 aa, synthetic construct
  REGION 1..107: note = VL Peptide
EIVLTQSPATLSLSPGERATLSCRASQSVSSYLAWYQQKPGQAPRLLIYDASNRATGIPARFSGSGSGTDFTLTISSLEPEDFAVYYCQQLNNFPPTFGGGTKVEIK

SEQ ID NO: 32 - DNA, 321 bp, synthetic construct
  misc_feature 1..321: note = VL DNA
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagctcaacaatttccctcctacttttggcggagggaccaaggttgagatcaaa

SEQ ID NO: 33 - protein, 291 aa, Homo sapiens
MTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 34 - protein, 230 aa, synthetic construct
  REGION 1..230: note = Human ASGR1 Extracellular domain
QNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 35 - protein, 256 aa, synthetic construct
  REGION 1..256: note = His-Avi-ASGR1 ECD
HHHHHHGGSGLNDIFEAQKIEWHEGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

SEQ ID NO: 36 - protein, 10 aa, synthetic construct
  REGION 1..10: note = p31 peptide
YVRPLWVRME

SEQ ID NO: 37 - protein, 18 aa, synthetic construct
  REGION 1..18: note = GS Linker
GSGGGGSGGGGSGGGGSG

SEQ ID NO: 38 - protein, 14 aa, synthetic construct
  REGION 1..14: note = pGal-p31
GGTCYVRPLWVRME

SEQ ID NO: 39 - protein, 28 aa, synthetic construct
  REGION 1..28: note = Linker-p31
GSGGGGSGGGGSGGGGSGYVRPLWVRME

SEQ ID NO: 40 - protein, 33 aa, synthetic construct
  REGION 1..33: note = HLA-DQ-2.5 relevant, Alpha-gliadin 33-mer native
LQLQPFPQPQLPYPQPQLPYPQPQLPYPQPQPF

SEQ ID NO: 41 - protein, 33 aa, synthetic construct
  REGION 1..33: note = HLA-DQ-2.5 relevant, Alpha-gliadin 33-mer deamidated
LQLQPFPQPELPYPQPELPYPQPELPYPQPQPF

SEQ ID NO: 42 - protein, 18 aa, synthetic construct
  REGION 1..18: note = HLA-DQ-8 relevant, Alpha-gliadin
QQYPSGQGSFQPSQQNPQ

SEQ ID NO: 43 - protein, 12 aa, synthetic construct
  REGION 1..12: note = HLA-DQ-8 relevant, Omega-gliadin (wheat, U5UA46)
QPFPQPEQPFPW

SEQ ID NO: 44 - protein, 15 aa, synthetic construct
  REGION 1..15: note = Alpha-gliadin 15-mer fragment
ELQPFPQPELPYPQP

SEQ ID NO: 45 - protein, 15 aa, synthetic construct
  REGION 1..15: note = Gliadin with linker
GGGPQPQPFPSQQPY

SEQ ID NO: 46 - protein, 18 aa, synthetic construct
  REGION 1..18: note = Gliadin with linker and cysteine conjugation moiety
GCRGGGPQPQPFPSQQPY

SEQ ID NO: 47 - protein, 45 aa, synthetic construct
  REGION 1..45: note = Gliadin extended
PQPQPFPSQQPYLQLQPFPQPQLPYPQPQLPYPQPQLPYPQPQPF

SEQ ID NO: 48 - protein, 48 aa, synthetic construct
  REGION 1..48: note = Gliadin extended with cysteine conjugation moiety
GCGPQPQPFPSQQPYLQLQPFPQPQLPYPQPQLPYPQPQLPYPQPQPF

SEQ ID NO: 49 - protein, 45 aa, synthetic construct
  REGION 1..45: note = Gliadin deamidated extended
PQPQPFPSQQPYLQLQPFPQPELPYPQPELPYPQPELPYPQPQPF

SEQ ID NO: 50 - protein, 48 aa, synthetic construct
  REGION 1..48: note = Gliadin deamidated extended with cysteine conjugation moiety
GCGPQPQPFPSQQPYLQLQPFPQPELPYPQPELPYPQPELPYPQPQPF

SEQ ID NO: 51 - protein, 25 aa, synthetic construct
  REGION 1..25: note = DQ-8, Alpha-gliadin
FQQPQQQYPSGEGSFQPSQENPQAQ

SEQ ID NO: 52 - protein, 27 aa, synthetic construct
  REGION 1..27: note = DQ-8, Alpha-gliadin with cysteine conjugation moiety
GCFQQPQQQYPSGEGSFQPSQENPQAQ

SEQ ID NO: 53 - protein, 41 aa, synthetic construct
  REGION 1..41: note = DQ-8, Alpha-gliadin extended
FQQPQQQYPSGEGSFQPSQENPQAQGSVQPQQLPQFEEIRN

SEQ ID NO: 54 - protein, 43 aa, synthetic construct
  REGION 1..43: note = DQ-8, Alpha-gliadin extended with cysteine conjugation moiety
GCFQQPQQQYPSGEGSFQPSQENPQAQGSVQPQQLPQFEEIRN

SEQ ID NO: 55 - protein, 70 aa, synthetic construct
  REGION 1..70: note = Human Proinsulin 1-70
FVNQHLCGSHLVEALYLVCGERGFFYTPKTRREAEDLQVGQVELGGGPGAGSLQPLALEGSLQKRGIVEQ

SEQ ID NO: 56 - protein, 62 aa, synthetic construct
  REGION 1..62: note = Human Proinsulin 9-70
SHLVEALYLVCGERGFFYTPKTRREAEDLQVGQVELGGGPGAGSLQPLALEGSLQKRGIVEQ

SEQ ID NO: 57 - protein, 30 aa, synthetic construct
  REGION 1..30: note = Human Proinsulin 9-38
SHLVEALYLVCGERGFFYTPKTRREAEDLQ

SEQ ID NO: 58 - protein, 38 aa, synthetic construct
  REGION 1..38: note = Human Proinsulin 1-38
FVNQHLCGSHLVEALYLVCGERGFFYTPKTRREAEDLQ

SEQ ID NO: 59 - protein, 15 aa, synthetic construct
  REGION 1..15: note = Human Proinsulin 9-23
SHLVEALYLVCGERG

SEQ ID NO: 60 - protein, 27 aa, synthetic construct
  REGION 1..27: note = Human Proinsulin 45-71 (C13-A6)
GGGPGAGSLQPLALEGSLQKRGIVEQC

SEQ ID NO: 61 - protein, 11 aa, synthetic construct
  REGION 1..11: note = Human Proinsulin C24-A1
LALEGSLQKRG

SEQ ID NO: 62 - protein, 18 aa, synthetic construct
  REGION 1..18: note = Human Proinsulin C19-A3
GSLQPLALEGSLQKRGIV

SEQ ID NO: 63 - protein, 20 aa, synthetic construct
  REGION 1..20: note = Human Proinsulin C13-32
GGGPGAGSLQPLALEGSLQK

SEQ ID NO: 64 - protein, 28 aa, synthetic construct
  REGION 1..28: note = Human Proinsulin B9-C4
SHLVEALYLVCGERGFFYTPKTRREAED

SEQ ID NO: 65 - protein, 17 aa, synthetic construct
  REGION 1..17: note = Human Proinsulin C22-A5
QPLALEGSLQKRGIVEQ

SEQ ID NO: 66 - protein, 39 aa, synthetic construct
  REGION 1..39: note = Human IA-2 654-674
AEGPPEPSRVSSVSSQFSDAAQASPSSHSSTPSWCEEPA

SEQ ID NO: 67 - protein, 65 aa, synthetic construct
  REGION 1..65: note = Human IA-2 718-782
AYQAEPNTCATAQGEGNIKKNRHPDFLPYDHARIKLKVESSPSRSDYINASPIIEHDPRMPAYIA

SEQ ID NO: 68 - protein, 35 aa, synthetic construct
  REGION 1..35: note = Human IA-2 785-819
GPLSHTIADFWQMVWESGCTVIVMLTPLVEDGVKQ

SEQ ID NO: 69 - protein, 56 aa, synthetic construct
  REGION 1..56: note = Human IA-2 828-883
GASLYHVYEVNLVSEHIWCEDFLVRSFYLKNVQTQETRTLTQFHFLSWPAEGTPAS

SEQ ID NO: 70 - protein, 39 aa, synthetic construct
  REGION 1..39: note = Human IA-2 943-979
EHVRDQRPGLVRSKDQFEFALTAVAEEVNAILKALPQCG

SEQ ID NO: 71 - protein, 42 aa, synthetic construct
  REGION 1..42: note = MBP 1-39
GCASQKRPSQRHGSKYLATASTMDHARHGFLPRHRDTGILDS

SEQ ID NO: 72 - protein, 20 aa, synthetic construct
  REGION 1..20: note = MBP 13-32
KYLATASTMDHARHGFLPRH

SEQ ID NO: 73 - protein, 33 aa, synthetic construct
  REGION 1..33: note = MBP 76-106
SHGRTQDENPVVHFFKNIVTPRTPPPSQGKGCG

SEQ ID NO: 74 - protein, 16 aa, synthetic construct
  REGION 1..16: note = MBP 83-99
ENPWHFFKNIVTPRTP

SEQ ID NO: 75 - protein, 37 aa, synthetic construct
  REGION 1..37: note = MBP 102-136
SQGKGRGLSLSRFSWGAEGQRPGFGYGGRASDYKSCG

SEQ ID NO: 76 - protein, 19 aa, synthetic construct
  REGION 1..19: note = MBP 111-129
LSRFSWGAEGQRPGFGYGG

SEQ ID NO: 77 - protein, 25 aa, synthetic construct
  REGION 1..25: note = MBP 146-170
AQGTLSKIFKLGGRDSRSGSPMARR

SEQ ID NO: 78 - protein, 27 aa, synthetic construct
  REGION 1..27: note = MBP 146-170 with cysteine conjugation moiety
AQGTLSKIFKLGGRDSRSGSPMARRCG

SEQ ID NO: 79 - protein, 17 aa, synthetic construct
  REGION 1..17: note = MBP 83-99
ENPVVHFFKNIVTPRTP

SEQ ID NO: 80 - protein, 17 aa, synthetic construct
  REGION 1..17: note = MBP 82-98
DENPVVHFFKNIVTPRT

SEQ ID NO: 81 - protein, 18 aa, synthetic construct
  REGION 1..18: note = MBP 82-99
DENPVVHFFKNIVTPRTP

SEQ ID NO: 82 - protein, 25 aa, synthetic construct
  REGION 1..25: note = MBP 82-106
DENPVVHFFKNIVTPRTPPPSQGKG

SEQ ID NO: 83 - protein, 20 aa, synthetic construct
  REGION 1..20: note = MBP 87-106
VHFFKNIVTPRTPPPSQGKG

SEQ ID NO: 84 - protein, 25 aa, synthetic construct
  REGION 1..25: note = MBP 131-155
ASDYKSAHKGLKGVDAQGTLSKIFK

SEQ ID NO: 85 - protein, 42 aa, synthetic construct
  REGION 1..42: note = MBP 131-170 with cysteine conjugation moiety
ASDYKSAHKGFKGVDAQGTLSKIFKLGGRDSRSGSPMARRCG

SEQ ID NO: 86 - protein, 63 aa, synthetic construct
  REGION 1..63: note = MBP 76-136
SHGRTQDENPVVHFFKNIVTPRTPPPSQGKGRGLSLSRFSWGAEGQRPGFGYGGRASDYKSCG

SEQ ID NO: 87 - protein, 20 aa, synthetic construct
  REGION 1..20: note = MOG 1-20
GQFRVIGPRHPIRALVGDEV

SEQ ID NO: 88 - protein, 27 aa, synthetic construct
  REGION 1..27: note = MOG 1-27
GQFRVIGPRHPIRALVGDEVELPCRIS

SEQ ID NO: 89 - protein, 20 aa, synthetic construct
  REGION 1..20: note = MOG 35-55
MEVGWYRPPFSRWHLYRNGK

SEQ ID NO: 90 - protein, 31 aa, synthetic construct
  REGION 1..31: note = MOG 30-60
KNATGMEVGWYRSPFSRVVHLYRNGKDQDAE

SEQ ID NO: 91 - protein, 23 aa, synthetic construct
  REGION 1..23: note = MOG 34-56
GMEVGWYRSPFSRVVHLYRNGKD

SEQ ID NO: 92 - protein, 21 aa, synthetic construct
  REGION 1..21: note = MOG 35-55
MEVGWYRPPFSRVVHLYRNGK

SEQ ID NO: 93 - protein, 21 aa, synthetic construct
  REGION 1..21: note = MOG 35-55 (Mouse)
MEVGWYRSPFSRVVHLYRNGK

SEQ ID NO: 94 - protein, 30 aa, synthetic construct
  REGION 1..30: note = MOG 33-62
TGMEVGWYRPPFSRVVHLYRNGKDQDGDQA

SEQ ID NO: 95 - protein, 20 aa, synthetic construct
  REGION 1..20: note = MOG 11-30
PIRALVGDEVELPCRISPGK

SEQ ID NO: 96 - protein, 45 aa, synthetic construct
  REGION 1..45: note = MOG 18-62
DEVELPCRISPGKNATGMEVGWYRPPFSRVVHLYRNGKDQDGDQA

SEQ ID NO: 97 - protein, 20 aa, synthetic construct
  REGION 1..20: note = MOG 21-40
ELPCRISPGKNATGMEVGWY

SEQ ID NO: 98 - protein, 23 aa, synthetic construct
  REGION 1..23: note = MOG 64-86
EYRGRTELLKDAIGEGKVTLRIR

SEQ ID NO: 99 - protein, 60 aa, synthetic construct
  REGION 1..60: note = MOG 1-60
GQFRVIGPRHPIRALVGDEVELPCRISPGKNATGMEVGWYRPPFSRVVHLYRNGKDQDGD

SEQ ID NO: 100 - protein, 62 aa, synthetic construct
  REGION 1..62: note = MOG 1-62
GQFRVIGPRHPIRALVGDEVELPCRISPGKNATGMEVGWYRPPFSRVVHLYRNGKDQDGDQA

SEQ ID NO: 101 - protein, 18 aa, synthetic construct
  REGION 1..18: note = PLP 41-58
GTEKLIETYFSKNYQDYE

SEQ ID NO: 102 - protein, 18 aa, synthetic construct
  REGION 1..18: note = PLP 89-106
GFYTTGAVRQIFGDYKTT

SEQ ID NO: 103 - protein, 22 aa, synthetic construct
  REGION 1..22: note = PLP 95-116
AVRQIFGDYKTTICGKGLSATV

SEQ ID NO: 104 - protein, 20 aa, synthetic construct
  REGION 1..20: note = PLP 178-197
NTWTTCQSIAFPSKTSASIG

SEQ ID NO: 105 - protein, 20 aa, synthetic construct
  REGION 1..20: note = PLP 190-209
SKTSASIGSLCADARMYGVL

SEQ ID NO: 106 - protein, 16 aa, synthetic construct
  REGION 1..16: note = PLP 139-154
HCLGKWLGHPDKFVGI

SEQ ID NO: 107 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 108 - protein, 14 aa, synthetic construct
  REGION 1..14: note = Collagen II 1236-1249
LQYMRADQAAGGLR

SEQ ID NO: 109 - protein, 2530 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACLRTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATLATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAATEESTEGPSATEVPSASEEPSPSEVPFPSEEPSPSEEPFPSVRPFPSVELFPSEEPFPSKEPSPSEEPSASEEPYTPSPPVPSWTELPSSGEESGAPDVSGDFTGSGDVSGHLDFSGQLSGDRASGLPSGDLDSSGLTSTVGSGLPVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGLPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGISGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAAAPARSCAEEPCGAGTCKETEGHVICLCPPGYTGEHCNIDQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTVACGEPPVVEHARTFGQKKDRYEINSLVRYQCTEGFVQRHMPTIRCQPSGHWEEPQITCTDPTTYKRRLQKRSSRHPRRSRPSTAH

SEQ ID NO: 110 - protein, 20 aa, synthetic construct
  REGION 1..20: note = Aggrecan 84-103
VVLLVATEGRVRVNSAYQDK

SEQ ID NO: 111 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 112 - protein, 14 aa, synthetic construct
  REGION 1..14: note = Fibrinogen alpha chain 78-91
NQDFTNRINKLKNS

SEQ ID NO: 113 - protein, 9 aa, synthetic construct
  REGION 1..9: note = Fibrinogen alpha chain 717-725
LTQRGSVLR

SEQ ID NO: 114 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 115 - protein, 13 aa, synthetic construct
  REGION 1..13: note = Vimentin 66-78
SAVRLRSSVPGVR

SEQ ID NO: 116 - protein, 9 aa, synthetic construct
  REGION 1..9: note = Vimentin 447-455
VETRDGQVI

SEQ ID NO: 117 - protein, 475 aa, Homo sapiens [sequence omitted]

SEQ ID NO: 118 - protein, 408 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 119 - protein, 1939 aa, Homo sapiens
MTDAQMADFGAAAQYLRKSEKERLEAQTRPFDIRTECFVPDDKEEFVKAKILSREGGKVIAETENGKTVTVKEDQVLQQNPPKFDKIEDMAMLTFLHEPAVLFNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRGKKDNANANKGTLEDQIIQANPALEAFGNAKTVRNDNSSRFGKFIRIHFGATGKLASADIETYLLEKSRVIFQLKAERNYHIFYQILSNKKPELLDMLLVTNNPYDYAFVSQGEVSVASIDDSEELMATDSAFDVLGFTSEEKAGVYKLTGAIMHYGNMKFKQKQREEQAEPDGTEDADKSAYLMGLNSADLLKGLCHPRVKVGNEYVTKGQSVQQVYYSIGALAKAVYEKMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLYDNHLGKSNNFQKPRNIKGKQEAHFSLIHYAGTVDYNILGWLEKNKDPLNETVVALYQKSSLKLMATLFSSYATADTGDSGKSKGGKKKGSSFQTVSALHRENLNKLMTNLRTTHPHFVRCIIPNERKAPGVMDNPLVMHQLRCNGVLEGIRICRKGFPNRILYGDFRQRYRILNPVAIPEGQFIDSRKGTEKLLSSLDIDHNQYKFGHTKVFFKAGLLGLLEEMRDERLSRIITRMQAQARGQLMRIEFKKIVERRDALLVIQWNIRAFMGVKNWPWMKLYFKIKPLLKSAETEKEMATMKEEFGRIKETLEKSEARRKELEEKMVSLLQEKNDLQLQVQAEQDNLNDAEERCDQLIKNKIQLEAKVKEMNERLEDEEEMNAELTAKKRKLEDECSELKKDIDDLELTLAKVEKEKHATENKVKNLTEEMAGLDEIIAKLTKEKKALQEAHQQALDDLQVEEDKVNSLSKSKVKLEQQVDDLEGSLEQEKKVRMDLERAKRKLEGDLKLTQESIMDLENDKLQLEEKLKKKEFDINQQNSKIEDEQVLALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRVKLEEAQRSLNDFTTQRAKLQTENGELARQLEEKEALISQLTRGKLSYTQQMEDLKRQLEEEGKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNVHELEKVRKQLEVEKLELQSALEEAEASLEHEEGKILRAQLEFNQIKAEIERKLAEKDEEMEQAKRNHQRVVDSLQTSLDAETRSRNEVLRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMESDLTQLQSEVEEAVQECRNAEEKAKKAITDAAMMAEELKKEQDTSAHLERMKKNMEQTIKDLQHRLDEAEQIALKGGKKQLQKLEARVRELEGELEAEQKRNAESVKGMRKSERRIKELTYQTEEDKKNLLRLQDLVDKLQLKVKAYKRQAEEAEEQANTNLSKFRKVQHELDEAEERADIAESQVNKLRAKSRDIGAKQKMHDEE

SEQ ID NO: 120 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 121 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 122 - protein, 15 aa, synthetic construct
  REGION 1..15: note = Myeloperoxidase 447-461
RKIVGAMVQIITYRD

SEQ ID NO: 123 - protein, 20 aa, synthetic construct
  REGION 1..20: note = Myeloperoxidase 435-454
PRWDGERLYQEARKIVGAMV

SEQ ID NO: 124 - protein, 15 aa, synthetic construct
  REGION 1..15: note = Myeloperoxidase 409-423
MPELTSMHTLLLREH

SEQ ID NO: 125 - protein, 66 aa, synthetic construct
  REGION 1..66: note = Myeloperoxidase 409-474
MPELTSMHTLLLREHNRLATELKSLNPRWDGERLYQEARKIVGAMVQIITYRDYLPLVLGPTAMRK

SEQ ID NO: 126 - protein, 63 aa, synthetic construct
  REGION 1..63: note = Myeloperoxidase 279-341
VNCETSCVQQPPCFPLKIPPNDPRIKNQADCIPFFRSCPACPGSNITIRNQINALTSFVDASM

SEQ ID NO: 127 - protein, 69 aa, synthetic construct
  REGION 1..69: note = Myeloperoxidase 341-409
MVYGSEEPLARNLRNMSNQLGLLAVNQRFQDNGRALLPFDNLHDDPCLLTNRSARIPCFLAGDTRSSEM

SEQ ID NO: 128 - protein, 148 aa, synthetic construct
  REGION 1..148: note = Myeloperoxidase 598-745
GYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 129 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 130 - protein, 647 aa, Homo sapiens
MWRVCARRAQNVAPWAGLEARWTALQEVPGTPRVTSRSGPAPARRNSVTTGYGGVRALCGWTPSSGATPRNRLLLQLLGSPGRRYYSLPPHQKVPLPSLSPTMQAGTIARWEKKEGDKINEGDLIAEVETDKATVGFESLEECYMAKILVAEGTRDVPIGAIICITVGKPEDIEAFKNYTLDSSAAPTPQAAPAPTPAATASPPTPSAQAPGSSYPPHMQVLLPALSPTMTMGTVQRWEKKVGEKLSEGDLLAEIETDKATIGFEVQEEGYLAKILVPEGTRDVPLGTPLCIIVEKEADISAFADYRPTEVTDLKPQVPPPTPPPVAAVPPTPQPLAPTPSAPCPATPAGPKGRVFVSPLAKKLAVEKGIDLTQVKGTGPDGRITKKDIDSFVPSKVAPAPAAVVPPTGPGMAPVPTGVFTDIPISNIRRVIAQRLMQSKQTIPHYYLSIDVNMGEVLLVRKELNKILEGRSKISVNDFIIKASALACLKVPEANSSWMDTVIRQNHVVDVSVAVSTPAGLITPIVFNAHIKGVETIANDVVSLATKAREGKLQPHEFQGGTFTISNLGMFGIKNFSAIINPPQACILAIGASEDKLVPADNEKGFDVASMMSVTLSCDHRVVDGAVGAQWLAEFRKYLEKPITMLL

SEQ ID NO: 131 - protein, 14 aa, synthetic construct
  REGION 1..14: note = Pyruvate dehydrogenase complex E2 subunit 163-176 (250-262)
GDLLAEIETDKATI

SEQ ID NO: 132 - protein, 9 aa, synthetic construct
  REGION 1..9: note = Pyruvate dehydrogenase complex E2 subunit 159-167 (245-253)
KLSEGDLLA

SEQ ID NO: 133 - protein, 497 aa, Homo sapiens [sequence omitted]

SEQ ID NO: 134 - protein, 10 aa, synthetic construct
  REGION 1..10: note = Cytochrome P450 2D6 245-254
KAFLTQLDEL

SEQ ID NO: 135 - protein, 44 aa, synthetic construct
  REGION 1..44: note = Cytochrome P450 2D6 217-260
SGFLREVLNAVPVLLHIPALAGKVLRFQKAFLTQLDELLTEHRM

SEQ ID NO: 136 - protein, 44 aa, synthetic construct
  REGION 1..44: note = Cytochrome P450 2D6 305-348
AGMVTTSTTLAWGLLLMILHPDVQRRVQQEIDDVIGQVRRPEMG

SEQ ID NO: 137 - protein, 20 aa, synthetic construct
  REGION 1..20: note = Cytochrome P450 2D6 193-212
RRFEYDDPRFLRLLDLAQEG

SEQ ID NO: 138 - protein, 20 aa, synthetic construct
  REGION 1..20: note = Cytochrome P450 2D6 305-324
AGMVTTSTTLAWGLLLMILH

SEQ ID NO: 139 - protein, 20 aa, synthetic construct
  REGION 1..20: note = Cytochrome P450 2D6 313-332
TLAWGLLLMILHPDVQRRVQ

SEQ ID NO: 140 - protein, 20 aa, synthetic construct
  REGION 1..20: note = Cytochrome P450 2D6 393-412
TTLITNLSSVLKDEAVWEKP

SEQ ID NO: 141 - protein, 735 aa, synthetic construct [sequence omitted]

SEQ ID NO: 142 - protein, 598 aa, synthetic construct
  REGION 1..598: note = AAV2 Capsid Protein 2
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 143 - protein, 533 aa, synthetic construct
  REGION 1..533: note = AAV2 Capsid Protein 3
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 144 - protein, 736 aa, synthetic construct
  REGION 1..736: note = AAV9 Capsid Protein 1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 145 - protein, 599 aa, synthetic construct
  REGION 1..599: note = AAV9 Capsid Protein 2
TAPGKKRPVEQSPQEPDSSSGIGKSGQQPAKKRLNFGQTGDSESVPDPQPLGEPPEAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 146 - protein, 534 aa, synthetic construct
  REGION 1..534: note = AAV9 Capsid Protein 3
MASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 147 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 148 - protein, 529 aa, Homo sapiens [sequence omitted]

SEQ ID NO: 149 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 150 - protein, 323 aa, Homo sapiens
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 151 - protein, 405 aa, Homo sapiens
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 152 - protein, 1247 aa, Homo sapiens
MMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 153 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 154 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 155 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 156 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 157 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 158 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 159 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 160 - protein, 19 aa, synthetic construct
  REGION 1..19: note = Collagen alpha-1(II) 662-678
SGFQGLPGPPGPPGEGGGK

SEQ ID NO: 161 - protein, 19 aa, synthetic construct
  REGION 1..19: note = Collagen alpha-1(II) 459-478
GIAGFKGEQGKGEPGPAGP

SEQ ID NO: 162 - protein, 12 aa, synthetic construct
  REGION 1..12: note = Collagen alpha-1(II) 461-473
AGFKGEQGKGEP

SEQ ID NO: 163 - protein, 15 aa, synthetic construct
  REGION 1..15: note = Collagen alpha-1(II) 471-485
GEPGPAGPQGAPGPA

SEQ ID NO: 164 - protein, 21 aa, synthetic construct
  REGION 1..21: note = Collagen alpha-1(II) 450-470
GPKGQTGEPGIAGFKGEQGPK

SEQ ID NO: 165 - protein, 9 aa, synthetic construct
  REGION 1..9: note = Collagen alpha-1(II) 595-603
ARGQPGVMG

SEQ ID NO: 166 - protein, 20 aa, synthetic construct
  REGION 1..20: note = Collagen alpha-1(II) 455-474
TGEPGIAGFKGEQGPKGEPG

SEQ ID NO: 167 - protein, 14 aa, synthetic construct
  REGION 1..14: note = Collagen alpha-1(II) 498-511
GGVGPIGPPGERGA

SEQ ID NO: 168 - protein, 18 aa, synthetic construct
  REGION 1..18: note = Collagen alpha-1(II) 1133-1150
RGFTGLQGLPGPPGPSGD

SEQ ID NO: 169 - protein, 9 aa, synthetic construct
  REGION 1..9: note = Collagen alpha-1(II) 1427-1435
SRFTYTALK

SEQ ID NO: 170 - protein, 63 aa, synthetic construct
  REGION 1..63: note = Collagen alpha-1(II) 378-440
GPTGARGPEGAQGPRGEPGTPGSPGPAGASGNPGTDGIPGAKGSAGAPGIAGAPGFPGPRGPP

SEQ ID NO: 171 - protein, 13 aa, synthetic construct
  REGION 1..13: note = Collagen alpha-1(II) 1350-1362
VEIRAEGNSRFTY

SEQ ID NO: 172 - protein, 13 aa, synthetic construct
  REGION 1..13: note = Collagen alpha-1(II) 1237-1249
QYMRADQAAGGLR

SEQ ID NO: 173 - protein, 15 aa, synthetic construct
  REGION 1..15: note = Collagen alpha-1(II) 511-525
APGNRGFPGQDGLAG

SEQ ID NO: 174 - protein, 9 aa, synthetic construct
  REGION 1..9: note = Collagen alpha-1(II) 756-764
GPIGPPGPA

SEQ ID NO: 175 - protein, 45 aa, synthetic construct
  REGION 1..45: note = Fibrinogen-a 51-95
DWPFCSDEDWNYKCPSGCRMKGLIDEVNQDFTNRINKLKNSLFEY

SEQ ID NO: 176 - protein, 15 aa, synthetic construct
  REGION 1..15: note = Fibrinogen-a 138-152
IEVLKRKVIEKVQHI

SEQ ID NO: 177 - protein, 15 aa, synthetic construct
  REGION 1..15: note = Fibrinogen-a 171-185
VDIDIKIRSCRGSCS

SEQ ID NO: 178 - protein, 15 aa, synthetic construct
  REGION 1..15: note = Fibrinogen-a 201-215
QKQLEQVIAKDLLPS

SEQ ID NO: 179 - protein, 15 aa, synthetic construct
  REGION 1..15: note = Fibrinogen-a 300-314
SGPGSTGNRNPGSSG

SEQ ID NO: 180 - protein, 15 aa, synthetic construct
  REGION 1..15: note = Fibrinogen-a 347-361
QNPGSPRPGSTGTWN

SEQ ID NO: 181 - protein, 15 aa, synthetic construct
  REGION 1..15: note = Fibrinogen-a 363-377
GSSERGSAGHWTSES

SEQ ID NO: 182 - protein, 15 aa, synthetic construct
  REGION 1..15: note = Fibrinogen-a 420-434
VSPGTRREYHTEKLV

SEQ ID NO: 183 - protein, 15 aa, synthetic construct
  REGION 1..15: note = Fibrinogen-a 438-452
GDKELRTGKEKVTSG

SEQ ID NO: 184 - protein, 15 aa, synthetic construct
  REGION 1..15: note = Fibrinogen-a 456-470
TTRRSCSKTVTKTVI

SEQ ID NO: 185 - protein, 15 aa, synthetic construct
  REGION 1..15: note = Fibrinogen-a 542-556
SETESRGSESGIFTN

SEQ ID NO: 186 - protein, 9 aa, synthetic construct
  REGION 1..9: note = Fibrinogen-a 717-725
LTQRGSVLR

SEQ ID NO: 187 - protein, 15 aa, synthetic construct
  REGION 1..15: note = Fibrinogen-a 737-751
YAEYHFRVGSEAEGY

SEQ ID NO: 188 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 189 - protein, 9 aa, synthetic construct
  REGION 1..9: note = Fibrinogen beta chain 433-441
WYNRCHAAN

SEQ ID NO: 190 - protein, 13 aa, synthetic construct
  REGION 1..13: note = Fibrinogen beta chain 69-81
GGYRARPAKAAAT

SEQ ID NO: 191 - protein, 24 aa, synthetic construct
  REGION 1..24: note = Vimentin 26-49
SSRSYVTTSTRTYSLGSALRPSTS

SEQ ID NO: 192 - protein, 38 aa, synthetic construct
  REGION 1..38: note = Vimentin 51-88
SLYASSPGGVYATRSSAVRLRSSVPGVRLLQDSVDFSL

SEQ ID NO: 193 - protein, 7 aa, synthetic construct
  REGION 1..7: note = Vimentin 116-122
NYIDKVR

SEQ ID NO: 194 - protein, 9 aa, synthetic construct
  REGION 1..9: note = Vimentin 130-138
ILLAELEQL

SEQ ID NO: 195 - protein, 17 aa, synthetic construct
  REGION 1..17: note = Vimentin 226-242
SLQEEIAFLKKLHEEEI

SEQ ID NO: 196 - protein, 17 aa, synthetic construct
  REGION 1..17: note = Vimentin 371-387
NMKEEMARHLREYQDLL

SEQ ID NO: 197 - protein, 29 aa, synthetic construct
  REGION 1..29: note = Vimentin 415-443
LPNFSSLNLRETNLDSLPLVDTHSKRTLL

SEQ ID NO: 198 - protein, 9 aa, synthetic construct
  REGION 1..9: note = Vimentin 447-455
VETRDGQVI

SEQ ID NO: 199 - protein, 33 aa, synthetic construct
  REGION 1..33: note = Aggrecan 32-64
SIPQPSPLRVLLGTSLTIPCYFIDPMHPVTTAP

SEQ ID NO: 200 - protein, 15 aa, synthetic construct
  REGION 1..15: note = Aggrecan 68-82
PLAPRIKWSRVSKEK

SEQ ID NO: 201 - protein, 35 aa, synthetic construct
  REGION 1..35: note = Aggrecan 76-110
SRVSKEKEVVLLVATEGRVRVNSAYQDKVSLPNYP

SEQ ID NO: 202 - protein, 15 aa, synthetic construct
  REGION 1..15: note = Aggrecan 116-130
ATLEVQSLRSNDSGV

SEQ ID NO: 203 - protein, 30 aa, synthetic construct
  REGION 1..30: note = Aggrecan 140-169
EDSEATLEVVVKGIVFHYRAISTRYTLDFD

SEQ ID NO: 204 - protein, 17 aa, synthetic construct
  REGION 1..17: note = Aggrecan 161-177
STRYTLDFDRAQRACLQ

SEQ ID NO: 205 - protein, 15 aa, synthetic construct
  REGION 1..15: note = Aggrecan 174-188
ACLQNSAIIATPEQL

SEQ ID NO: 206 - protein, 16 aa, synthetic construct
  REGION 1..16: note = Aggrecan 200-215
DAGWLADQTVRYPIHT

SEQ ID NO: 207 - protein, 20 aa, synthetic construct
  REGION 1..20: note = Aggrecan 225-244
DEFPGVRTYGIRDTNETYDV

SEQ ID NO: 208 - protein, 18 aa, synthetic construct
  REGION 1..18: note = Aggrecan 252-269
EGEVFYATSPEKFTFQEA

SEQ ID NO: 209 - protein, 28 aa, synthetic construct
  REGION 1..28: note = Aggrecan 287-314
YLAWQAGMDMCSAGWLADRSVRYPISKA

SEQ ID NO: 210 - protein, 15 aa, synthetic construct
  REGION 1..15: note = Aggrecan 341-355
SSRYDAICYTGEDFV

SEQ ID NO: 211 - protein, 20 aa, synthetic construct
  REGION 1..20: note = Aggrecan 520-539
GYEQCDAGWLRDQTVRYPIV

SEQ ID NO: 212 - protein, 18 aa, synthetic construct
  REGION 1..18: note = Aggrecan 553-570
PGVRTYGVRPSTETYDVY

SEQ ID NO: 213 - protein, 16 aa, synthetic construct
  REGION 1..16: note = Aggrecan 568-583
DVYCFVDRLEGEVFFA

SEQ ID NO: 214 - protein, 13 aa, synthetic construct
  REGION 1..13: note = Aggrecan 589-601
FTFQEALEFCESH

SEQ ID NO: 215 - protein, 17 aa, synthetic construct
  REGION 1..17: note = Aggrecan 620-636
KCYAGWLADGSLRYPIV

SEQ ID NO: 216 - protein, 9 aa, synthetic construct
  REGION 1..9: note = Aggrecan 666-674
SRHHAFCFR

SEQ ID NO: 217 - protein, 12 aa, synthetic construct
  REGION 1..12: note = Aggrecan 714-725
VEEETTAVPSGE

SEQ ID NO: 218 - protein, 14 aa, synthetic construct
  REGION 1..14: note = Aggrecan 866-879
VSGDFTGSGDVSGH

SEQ ID NO: 219 - protein, 11 aa, synthetic construct
  REGION 1..11: note = Aggrecan 988-998
GVEDISGLPSG

SEQ ID NO: 220 - protein, 12 aa, synthetic construct
  REGION 1..12: note = Aggrecan 1056-1067
EVLETAAPGVED

SEQ ID NO: 221 - protein, 15 aa, synthetic construct
  REGION 1..15: note = Aggrecan 1352-1366
LSGLPSGGEVLEISV

SEQ ID NO: 222 - protein, 14 aa, synthetic construct
  REGION 1..14: note = Aggrecan 1645-1658
STLVEVVTASTASE

SEQ ID NO: 223 - protein, 13 aa, synthetic construct
  REGION 1..13: note = Aggrecan 1892-1904
PEFSGLPSGIAEV

SEQ ID NO: 224 - protein, 14 aa, synthetic construct
  REGION 1..14: note = Aggrecan 1785-1798
LSGETSGVPDLSGQ

SEQ ID NO: 225 - protein, 15 aa, synthetic construct
  REGION 1..15: note = Aggrecan 1805-1819
GAYYGSGTPSSFPTV

SEQ ID NO: 226 - protein, 14 aa, synthetic construct
  REGION 1..14: note = Aggrecan 1885-1898
VSGESSRAEIGSSL

SEQ ID NO: 227 - protein, 14 aa, synthetic construct
  REGION 1..14: note = Aggrecan 1937-1950
EGPSGILELSGAHS

SEQ ID NO: 228 - protein, 14 aa, synthetic construct
  REGION 1..14: note = Aggrecan 1961-1974
GFLDLSGLQSGLIE

SEQ ID NO: 229 - protein, 14 aa, synthetic construct
  REGION 1..14: note = Aggrecan 2018-2031
ITSEFVEGVTEPTI

SEQ ID NO: 230 - protein, 15 aa, synthetic construct
  REGION 1..15: note = Aggrecan 2104-2118
DSGSPDLSETTSAFH

SEQ ID NO: 231 - protein, 13 aa, synthetic construct
  REGION 1..13: note = Aggrecan 2189-2201
QLGVVISTSIPES

SEQ ID NO: 232 - protein, 15 aa, synthetic construct
  REGION 1..15: note = Aggrecan 2282-2296
ETWVDAERRCREQQS

SEQ ID NO: 233 - protein, 18 aa, synthetic construct
  REGION 1..18: note = Aggrecan 2351-2368
QQSHLSSIVTPEEQEFVN

SEQ ID NO: 234 - protein, 15 aa, synthetic construct
  REGION 1..15: note = Aggrecan 2507-2521
TTYKRRLQKRSSRHP

SEQ ID NO: 235 - protein, 16 aa, synthetic construct
  REGION 1..16: note = Aggrecan 2513-2528
LQKRSSRHPRRSRPST

SEQ ID NO: 236 - protein, 24 aa, synthetic construct
  REGION 1..24: note = Aggrecan 2516-2530
RSSRHPRRSRPSTAHRRSRPSTAH

SEQ ID NO: 237 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 238 - protein, 37 aa, synthetic construct
  REGION 1..37: note = alpha enolase 4-40
LKIHAREIFDSRGNPTVEVDLFTSKGLFRAAVPSGAS

SEQ ID NO: 239 - protein, 15 aa, synthetic construct
  REGION 1..15: note = alpha enolase 326-340
KRIAKAVNEKSCNCL

SEQ ID NO: 240 - protein, 15 aa, synthetic construct
  REGION 1..15: note = SS-B (La) 19-33
HQIEYYFGDFNLPRD

SEQ ID NO: 241 - protein, 27 aa, synthetic construct
  REGION 1..27: note = SS-B (La) 46-72
WVPLEIMIKFNRLNRLTTDFNVIVEAL

SEQ ID NO: 242 - protein, 30 aa, synthetic construct
  REGION 1..30: note = SS-B (La) 151-180
KGSIFVVFDSIESAKKFVETPGQKYKETDL

SEQ ID NO: 243 - protein, 18 aa, synthetic construct
  REGION 1..18: note = SS-B (La) 211-228
QEAKQKLEEDAEMKSLEE

SEQ ID NO: 244 - protein, 15 aa, synthetic construct
  REGION 1..15: note = SS-B (La) 244-258
TCREDLHILFSNHGE

SEQ ID NO: 245 - protein, 26 aa, synthetic construct
  REGION 1..26: note = SS-B (La) 304-329
EVLEGEVEKEALKKIIEDQQESLNKW

SEQ ID NO: 246 - protein, 22 aa, synthetic construct
  REGION 1..22: note = a-synuclein 2-23
DVFMKGLSKAKEGVVAAAEKTK

SEQ ID NO: 247 - protein, 15 aa, synthetic construct
  REGION 1..15: note = a-synuclein 32-46
KTKEGVLYVGSKTKE

SEQ ID NO: 248 - protein, 30 aa, synthetic construct
  REGION 1..30: note = a-synuclein 56-85
AEKTKEQVTNVGGAVVTGVTAVAQKTVEGA

SEQ ID NO: 249 - protein, 30 aa, synthetic construct
  REGION 1..30: note = a-synuclein 81-110
TVEGAGSIAAATGFVKKDQLGKNEEGAPQE

SEQ ID NO: 250 - protein, 25 aa, synthetic construct
  REGION 1..25: note = a-synuclein 116-140
MPVDPDNEAYEMPSEEGYQDYEPEA

SEQ ID NO: 251 - protein, 1935 aa, Homo sapiens
MGDSEMAVFGAAAPYLRKSEKERLEAQTRPFDLKKDVFVPDDKQEFVKAKIVSREGGKVTAETEYGKTVTVKEDQVMQQNPPKFDKIEDMAMLTFLHEPAVLYNLKDRYGSWMIYTYSGLFCVTVNPYKWLPVYTPEVVAAYRGKKRSEAPPHIFSISDNAYQYMLTDRENQSILITGESGAGKTVNTKRVIQYFAVIAAIGDRSKKDQSPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDAEELMATDNAFDVLGFTSEEKNSMYKLTGAIMHFGNMKFKLKQREEQAEPDGTEEADKSAYLMGLNSADLLKGLCHPRVKVGNEYVTKGQNVQQVIYATGALAKAVYERMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLFDNHLGKSANFQKPRNIKGKPEAHFSLIHYAGIVDYNIIGWLQKNKDPLNETVVGLYQKSSLKLLSTLFANYAGADAPIEKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLSSLDIDHNQYKFGHTKVFFKAGLLGLLEEMRDERLSRIITRIQAQSRGVLARMEYKKLLERRDSLLVIQWNIRAFMGVKNWPWMKLYFKIKPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAKVEKEKHATENKVKNLTEEMAGLDEIIAKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQEAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSSGKTIHELEKVRKQLEAEKMELQSALEEAEASLEHEEGKILRAQLEFNQIKAEIERKLAEKDEEMEQAKRNHLRVVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGTKGLNEE

SEQ ID NO: 252 - protein, 18 aa, synthetic construct
  REGION 1..18: note = Myosin 6 1906-1923
AEERADIAESQVNKLRAK

SEQ ID NO: 253 - protein, 18 aa, synthetic construct
  REGION 1..18: note = Myosin 6 1828-1845
AEQKRNAESVKGMRKSER

SEQ ID NO: 254 - protein, 83 aa, synthetic construct
  REGION 1..83: note = Myosin 6 1411-1493
CSSLEKTKHRLQNEIEDLMVDVERSNAAAAALDKKQRNFDKILAEWKQKYEESQSELESSQKEARSLSTELFKLKNAYEESLE

SEQ ID NO: 255 - protein, 31 aa, synthetic construct
  REGION 1..31: note = Myosin 6 1554-1584
EEGKILRAQLEFNQIKAEIERKLAEKDEEME

SEQ ID NO: 256 - protein, 12 aa, synthetic construct
  REGION 1..12: note = Myosin 6 1170-1181
EFQKMRRDLEEA

SEQ ID NO: 257 - protein, 18 aa, synthetic construct
  REGION 1..18: note = Myosin 6 1619-1636
EGDLNEMEIQLSHANRMA

SEQ ID NO: 258 - protein, 44 aa, synthetic construct
  REGION 1..44: note = Myosin 6 1671-1714
IAIVERRNNLLQAELEELRAVVEQTERSRKLAEQELIETSERVQ

SEQ ID NO: 259 - protein, 15 aa, synthetic construct
  REGION 1..15: note = Myosin 6 450-464
KQPRQYFIGVLDIAG

SEQ ID NO: 260 - protein, 25 aa, synthetic construct
  REGION 1..25: note = Myosin 6 1801-1819
LEQEKKVRMDLERAKRKLEGDLKLT

SEQ ID NO: 261 - protein, 31 aa, synthetic construct
  REGION 1..31: note = Myosin 6 1333-1363
LQSARHDCDLLREQYEEETEAKAELQRVLSK

SEQ ID NO: 262 - protein, 31 aa, synthetic construct
  REGION 1..31: note = Myosin 6 1867-1897
LVDKLQLKVKAYKRQAEEAEEQANTNLSKFR

SEQ ID NO: 263 - protein, 66 aa, synthetic construct
  REGION 1..66: note = Myosin 6 1749-1814
RNAEEKAKKAITDAAMMAEELKKEQDTSAHLERMKKNMEQTIKDLQHRLDEAEQIALKGGKKQLQK

SEQ ID NO: 264 - protein, 15 aa, synthetic construct
  REGION 1..15: note = Myosin 6 1142-1156
RSDLSRELEEISERL

SEQ ID NO: 265 - protein, 18 aa, synthetic construct
  REGION 1..18: note = Myosin 6 1372-1389
RTKYETDAIQRTEELEEA

SEQ ID NO: 266 - protein, 18 aa, synthetic construct
  REGION 1..18: note = Myosin 6 1710-1727
SERVQLLHSQNTSLINQK

SEQ ID NO: 267 - protein, 18 aa, synthetic construct
  REGION 1..18: note = Myosin 6 1645-1662
SLQSLLKDTQIQLDDAVR

SEQ ID NO: 268 - protein, 15 aa, synthetic construct
  REGION 1..15: note = Myosin 6 689-703
VMHQLRCNGVLEGIR

SEQ ID NO: 269 - protein, 18 aa, synthetic construct
  REGION 1..18: note = Myosin 6 1593-1610
VVDSLQTSLDAETRSRNE

SEQ ID NO: 270 - protein, 9 aa, synthetic construct
  REGION 1..9: note = Myosin 6 111-119
YTYSGLFCV

SEQ ID NO: 271 - protein, 18 aa, synthetic construct
  REGION 1..18: note = Pyruvate dehydrogenase complex E2 subunit 159-176 (245-262)
KLSEGDLLAEIETDKATI

SEQ ID NO: 272 - protein, 12 aa, synthetic construct
  REGION 1..12: note = Collagen alpha-1(II) 461-473 Carbamylated(K4)
  SITE 4: note = MISC_FEATURE - Carbamylated
AGFKGEQGKGEP

SEQ ID NO: 273 - protein, 20 aa, synthetic construct
  REGION 1..20: note = Collagen alpha-1(II) 455-474 HYL(P4, P19)
  SITE 4: note = MISC_FEATURE - Hydroxylated
  SITE 19: note = MISC_FEATURE - Hydroxylated
TGEPGIAGFKGEQGPKGEPG

SEQ ID NO: 274 - protein, 13 aa, synthetic construct
  REGION 1..13: note = Collagen alpha-1(II) 1237-1249 CITR(R4)
  SITE 4: note = MISC_FEATURE - Citrullinated
QYMRADQAAGGLR

SEQ ID NO: 275 - protein, 15 aa, synthetic construct
  REGION 1..15: note = Collagen alpha-1(II) 511-525 CITR(R5)
  SITE 5: note = MISC_FEATURE - Citrullinated
APGNRGFPGQDGLAG

SEQ ID NO: 276 - protein, 9 aa, synthetic construct
  REGION 1..9: note = Collagen alpha-1(II) 756-764 HYL(P6)
  SITE 6: note = MISC_FEATURE - Hydroxylated
GPIGPPGPA

SEQ ID NO: 277 - protein, 15 aa, synthetic construct
  REGION 1..15: note = Fibrinogen-a 138-152 CITR(R6)
  SITE 6: note = MISC_FEATURE - Citrullinated
IEVLKRKVIEKVQHI

SEQ ID NO: 278 - protein, 15 aa, synthetic construct
  REGION 1..15: note = Fibrinogen-a 456-470 CITR(R3, R4)
  REGION 3..4: note = MISC_FEATURE - Citrullinated
TTRRSCSKTVTKTVI

SEQ ID NO: 279 - protein, 9 aa, synthetic construct
  REGION 1..9: note = Fibrinogen-a 717-725 CITR(R4)
  SITE 4: note = MISC_FEATURE - Citrullinated
LTQRGSVLR

SEQ ID NO: 280 - protein, 15 aa, synthetic construct
  REGION 1..15: note = Fibrinogen-a 737-751 CITR(R7)
  SITE 7: note = MISC_FEATURE - Citrullinated
YAEYHFRVGSEAEGY

SEQ ID NO: 281 - protein, 9 aa, synthetic construct
  REGION 1..9: note = Vimentin 447-455 CITR(R4)
  SITE 4: note = MISC_FEATURE - Citrullinated
VETRDGQVI

SEQ ID NO: 282 - protein, 17 aa, synthetic construct
  REGION 1..17: note = Aggrecan 161-177 CITR(R3, R10, R13)
  SITE 3: note = MISC_FEATURE - Citrullinated
  SITE 10: note = MISC_FEATURE - Citrullinated
  SITE 13: note = MISC_FEATURE - Citrullinated
STRYTLDFDRAQRACLQ

SEQ ID NO: 283 - protein, 16 aa, synthetic construct
  REGION 1..16: note = Aggrecan 200-215 CITR(R11)
  SITE 11: note = MISC_FEATURE - Citrullinated
DAGWLADQTVRYPIHT

SEQ ID NO: 284 - protein, 20 aa, synthetic construct
  REGION 1..20: note = Aggrecan 225-244 CITR(R7, R12)
  SITE 7: note = MISC_FEATURE - Citrullinated
  SITE 12: note = MISC_FEATURE - Citrullinated
DEFPGVRTYGIRDTNETYDV

SEQ ID NO: 285 - protein, 20 aa, synthetic construct
  REGION 1..20: note = Aggrecan 520-539 CITR(R11, R16)
  SITE 11: note = MISC_FEATURE - Citrullinated
  SITE 16: note = MISC_FEATURE - Citrullinated
GYEQCDAGWLRDQTVRYPIV

SEQ ID NO: 286 - protein, 18 aa, synthetic construct
  REGION 1..18: note = Aggrecan 553-570 CITR(R4, R9)
  SITE 4: note = MISC_FEATURE - Citrullinated
  SITE 9: note = MISC_FEATURE - Citrullinated
PGVRTYGVRPSTETYDVY

SEQ ID NO: 287 - protein, 16 aa, synthetic construct
  REGION 1..16: note = Aggrecan 568-583 CITR(R8)
  SITE 8: note = MISC_FEATURE - Citrullinated
DVYCFVDRLEGEVFFA

SEQ ID NO: 288 - protein, 17 aa, synthetic construct
  REGION 1..17: note = Aggrecan 620-636 CITR(R13)
  SITE 13: note = MISC_FEATURE - Citrullinated
KCYAGWLADGSLRYPIV

SEQ ID NO: 289 - protein, 26 aa, synthetic construct
EVQLLESGGGLVQPGGSLRLSCAASG

SEQ ID NO: 290 - protein, 14 aa, synthetic construct
WVRQAPGKGLEWVS

SEQ ID NO: 291 - protein, 30 aa, synthetic construct
RFTISRDNSKNTLYLQMNSLRAEDTAVYYC

SEQ ID NO: 292 - protein, 11 aa, synthetic construct
WGRGTLVTVSS

SEQ ID NO: 293 - DNA, 78 bp, synthetic construct
  source 1..78: mol_type = other DNA
gaggtgcagctgttggagtctgggggaggcttggtacagcctggggggtccctgagactctcctgtgcagcctctgga

SEQ ID NO: 294 - DNA, 42 bp, synthetic construct
  source 1..42: mol_type = other DNA
tgggtccgccaggctccagggaaggggctggagtgggtctca

SEQ ID NO: 295 - DNA, 90 bp, synthetic construct
  source 1..90: mol_type = other DNA
cggttcaccatctccagagacaattccaagaacacgctgtatctgcaaatgaacagcctgagagccgaggacacggcggtgtactactgc

SEQ ID NO: 296 - DNA, 33 bp, synthetic construct
  source 1..33: mol_type = other DNA
tgggggagaggtaccttggtcaccgtctcctca

SEQ ID NO: 297 - protein, 9 aa, synthetic construct
FTFSSYAMS

SEQ ID NO: 298 - protein, 17 aa, synthetic construct
AISGSGGSTYYADSVKG

SEQ ID NO: 299 - protein, 15 aa, synthetic construct
AKLGTTVSGGHYFDL

SEQ ID NO: 300 - DNA, 27 bp, synthetic construct
  source 1..27: mol_type = other DNA
ttcacctttagcagctatgccatgagc

SEQ ID NO: 301 - DNA, 51 bp, synthetic construct
  source 1..51: mol_type = other DNA
gctattagtggtagtggtggtagcacatactacgcagactccgtgaagggc

SEQ ID NO: 302 - DNA, 45 bp, synthetic construct
  source 1..45: mol_type = other DNA
gccaagttaggaaccacagtaagcggtggtcactacttcgaccta

SEQ ID NO: 303 - protein, 122 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKLGTTVSGGHYFDLWGRGTLVTVSS

SEQ ID NO: 304 - protein, 366 aa, synthetic construct
GAGGTGCAGCTGTTGGAGTCTGGGGGAGGCTTGGTACAGCCTGGGGGGTCCCTGAGACTCTCCTGTGCAGCCTCTGGATTCACCTTTAGCAGCTATGCCATGAGCTGGGTCCGCCAGGCTCCAGGGAAGGGGCTGGAGTGGGTCTCAGCTATTAGTGGTAGTGGTGGTAGCACATACTACGCAGACTCCGTGAAGGGCCGGTTCACCATCTCCAGAGACAATTCCAAGAACACGCTGTATCTGCAAATGAACAGCCTGAGAGCCGAGGACACGGCGGTGTACTACTGCGCCAAGTTAGGAACCACAGTAAGCGGTGGTCACTACTTCGACCTATGGGGGAGAGGTACCTTGGTCACCGTCTCCTCA

SEQ ID NO: 305 - protein, 23 aa, synthetic construct
EIVLTQSPATLSLSPGERATLSC

SEQ ID NO: 306 - protein, 15 aa, synthetic construct
WYQQKPGQAPRLLIY

SEQ ID NO: 307 - protein, 32 aa, synthetic construct; identical to SEQ ID NO: 19

SEQ ID NO: 308 - protein, 10 aa, synthetic construct
FGGGTKVEIK

SEQ ID NO: 309 - DNA, 69 bp, synthetic construct; identical to SEQ ID NO: 21
  source 1..69: mol_type = other DNA

SEQ ID NO: 310 - DNA, 45 bp, synthetic construct; identical to SEQ ID NO: 22
  source 1..45: mol_type = other DNA

SEQ ID NO: 311 - DNA, 96 bp, synthetic construct; identical to SEQ ID NO: 23
  source 1..96: mol_type = other DNA

SEQ ID NO: 312 - DNA, 30 bp, synthetic construct; identical to SEQ ID NO: 24
  source 1..30: mol_type = other DNA

SEQ ID NO: 313 - protein, 11 aa, synthetic construct
RASQSVSSYLA

SEQ ID NO: 314 - protein, 7 aa, synthetic construct
DASKRAT

SEQ ID NO: 315 - protein, 9 aa, synthetic construct
QQDSNHPLT

SEQ ID NO: 316 - DNA, 33 bp, synthetic construct; identical to SEQ ID NO: 28
  source 1..33: mol_type = other DNA

SEQ ID NO: 317 - DNA, 21 bp, synthetic construct
  source 1..21: mol_type = other DNA
gatgcatccaaaagggccact

SEQ ID NO: 318 - DNA, 27 bp, synthetic construct
  source 1..27: mol_type = other DNA
cagcaggacagtaaccaccctctcact

SEQ ID NO: 319 - protein, 107 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQDSNHPLTFGGGTKVEIK

SEQ ID NO: 320 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaaaagggccactggcatcccagccaggttcagtggcagtgggtctgggacagacttcactctcaccatcagcagcctagagcctgaagattttgcagtttattactgtcagcaggacagtaaccaccctctcacttttggcggagggaccaaggttgagatcaaa

SEQ ID NO: 321 - protein, 26 aa, synthetic construct
QVQLQESGPGLVKPSETLSLTCAVSG

SEQ ID NO: 322 - protein, 14 aa, synthetic construct
WIRQPPGKGLEWIG

SEQ ID NO: 323 - protein, 30 aa, synthetic construct
RVTISVDTSKNQFSLKLSSVTAADTAVYYC

SEQ ID NO: 324 - protein, 11 aa, synthetic construct
WGQGTTVTVSS

SEQ ID NO: 325 - DNA, 78 bp, synthetic construct
  source 1..78: mol_type = other DNA
caggtgcagctgcaggagtcgggcccaggactggtgaagccttcggagaccctgtccctcacctgcgctgtctctggt

SEQ ID NO: 326 - DNA, 42 bp, synthetic construct
  source 1..42: mol_type = other DNA
tggatccggcagcccccagggaaggggctggagtggattggg

SEQ ID NO: 327 - DNA, 90 bp, synthetic construct
  source 1..90: mol_type = other DNA
cgagtcaccatatcagtagacacgtccaagaaccagttctccctgaagctgagttctgtgaccgccgcagacacggcggtgtactactgc

SEQ ID NO: 328 - DNA, 33 bp, synthetic construct
  source 1..33: mol_type = other DNA
tggggccagggaacaactgtcaccgtctcctca

SEQ ID NO: 329 - protein, 10 aa, synthetic construct
YSISSGYYWG

SEQ ID NO: 330 - protein, 16 aa, synthetic construct
SIYHSGSTYYNPSLKS

SEQ ID NO: 331 - protein, 15 aa, synthetic construct
AREAGYPERGYGMDV

SEQ ID NO: 332 - DNA, 30 bp, synthetic construct
  source 1..30: mol_type = other DNA
tactccatcagcagtggttactactggggc

SEQ ID NO: 333 - DNA, 48 bp, synthetic construct
  source 1..48: mol_type = other DNA
agtatctatcatagtgggagcacctactacaacccgtccctcaagagt

SEQ ID NO: 334 - DNA, 45 bp, synthetic construct
  source 1..45: mol_type = other DNA
gccagagaagccggatacccagaaagaggatacggaatggacgta

SEQ ID NO: 335 - protein, 122 aa, synthetic construct
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAREAGYPERGYGMDVWGQGTTVTVSS

SEQ ID NO: 336 - protein, 366 aa, synthetic construct
CAGGTGCAGCTGCAGGAGTCGGGCCCAGGACTGGTGAAGCCTTCGGAGACCCTGTCCCTCACCTGCGCTGTCTCTGGTTACTCCATCAGCAGTGGTTACTACTGGGGCTGGATCCGGCAGCCCCCAGGGAAGGGGCTGGAGTGGATTGGGAGTATCTATCATAGTGGGAGCACCTACTACAACCCGTCCCTCAAGAGTCGAGTCACCATATCAGTAGACACGTCCAAGAACCAGTTCTCCCTGAAGCTGAGTTCTGTGACCGCCGCAGACACGGCGGTGTACTACTGCGCCAGAGAAGCCGGATACCCAGAAAGAGGATACGGAATGGACGTATGGGGCCAGGGAACAACTGTCACCGTCTCCTCA

SEQ ID NO: 337 - protein, 23 aa, synthetic construct
EIVMTQSPATLSVSPGERATLSC

SEQ ID NO: 338 - protein, 15 aa, synthetic construct
WYQQKPGQAPRLLIY

SEQ ID NO: 339 - protein, 32 aa, synthetic construct
GIPARFSGSGSGTEFTLTISSLQSEDFAVYYC

SEQ ID NO: 340 - protein, 10 aa, synthetic construct
FGGGTKVEIK

SEQ ID NO: 341 - DNA, 69 bp, synthetic construct
  source 1..69: mol_type = other DNA
gaaatagtgatgacgcagtctccagccaccctgtctgtgtctccaggggaaagagccaccctctcctgc

SEQ ID NO: 342 - DNA, 45 bp, synthetic construct
  source 1..45: mol_type = other DNA
tggtaccagcagaaacctggccaggctcccaggctcctcatctat

SEQ ID NO: 343 - DNA, 96 bp, synthetic construct
  source 1..96: mol_type = other DNA
ggtatcccagccaggttcagtggcagtgggtctgggacagagttcactctcaccatcagcagcctgcagtctgaagattttgcagtttattactgt

SEQ ID NO: 344 - DNA, 30 bp, synthetic construct; identical to SEQ ID NO: 24
  source 1..30: mol_type = other DNA

SEQ ID NO: 345 - protein, 11 aa, synthetic construct
RASQSVSSNLA

SEQ ID NO: 346 - protein, 7 aa, synthetic construct
GASTRAT

SEQ ID NO: 347 - protein, 9 aa, synthetic construct
QQYFHHPFT

SEQ ID NO: 348 - DNA, 33 bp, synthetic construct
  source 1..33: mol_type = other DNA
agggccagtcagagtgttagcagcaacttagcc

SEQ ID NO: 349 - DNA, 21 bp, synthetic construct
  source 1..21: mol_type = other DNA
ggtgcatccaccagggccact

SEQ ID NO: 350 - DNA, 27 bp, synthetic construct
  source 1..27: mol_type = other DNA
cagcagtacttccaccaccctttcact

SEQ ID NO: 351 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYFHHPFTFGGGTKVEIK

SEQ ID NO: 352 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtacttccaccaccctttcacttttggcggagggaccaaggttgagatcaaa

SEQ ID NO: 353 - protein, 26 aa, synthetic construct
EVQLLESGGGLVQPGGSLRLSCAASG

SEQ ID NO: 354 - protein, 14 aa, synthetic construct
WVRQAPGKGLEWVS

SEQ ID NO: 355 - protein, 30 aa, synthetic construct; identical to SEQ ID NO: 291

SEQ ID NO: 356 - protein, 11 aa, synthetic construct
WGRGTLVTVSS

SEQ ID NO: 357 - DNA, 78 bp, synthetic construct; identical to SEQ ID NO: 293
  source 1..78: mol_type = other DNA

SEQ ID NO: 358 - DNA, 42 bp, synthetic construct; identical to SEQ ID NO: 294
  source 1..42: mol_type = other DNA

SEQ ID NO: 359 - DNA, 90 bp, synthetic construct; identical to SEQ ID NO: 295
  source 1..90: mol_type = other DNA

SEQ ID NO: 360 - DNA, 33 bp, synthetic construct; identical to SEQ ID NO: 296
  source 1..33: mol_type = other DNA

SEQ ID NO: 361 - protein, 9 aa, synthetic construct
FTFSNYAMS

SEQ ID NO: 362 - protein, 17 aa, synthetic construct
AISGSGGSTYHADSVKG

SEQ ID NO: 363 - protein, 14 aa, synthetic construct
ARDAPYRGGHYFDL

SEQ ID NO: 364 - DNA, 27 bp, synthetic construct
  source 1..27: mol_type = other DNA
ttcacctttagcaattatgccatgagc

SEQ ID NO: 365 - DNA, 51 bp, synthetic construct
  source 1..51: mol_type = other DNA
gctattagtggtagtggtggtagcacataccacgcagactccgtgaagggc

SEQ ID NO: 366 - DNA, 42 bp, synthetic construct
  source 1..42: mol_type = other DNA
gccagagatgctccttacagaggtggtcactacttcgaccta

SEQ ID NO: 367 - protein, 121 aa, synthetic construct
EVQLLESGGGLVQPGGSLRLSCAASGFTFSNYAMSWVRQAPGKGLEWVSAISGSGGSTYHADSVKGRFTISRDNSKNTLYLQMNSLRAEDTAVYYCARDAPYRGGHYFDLWGRGTLVTVSS

SEQ ID NO: 368 - protein, 363 aa, synthetic construct
GAGGTGCAGCTGTTGGAGTCTGGGGGAGGCTTGGTACAGCCTGGGGGGTCCCTGAGACTCTCCTGTGCAGCCTCTGGATTCACCTTTAGCAATTATGCCATGAGCTGGGTCCGCCAGGCTCCAGGGAAGGGGCTGGAGTGGGTCTCAGCTATTAGTGGTAGTGGTGGTAGCACATACCACGCAGACTCCGTGAAGGGCCGGTTCACCATCTCCAGAGACAATTCCAAGAACACGCTGTATCTGCAAATGAACAGCCTGAGAGCCGAGGACACGGCGGTGTACTACTGCGCCAGAGATGCTCCTTACAGAGGTGGTCACTACTTCGACCTATGGGGGAGAGGTACCTTGGTCACCGTCTCCTCA

SEQ ID NO: 369 - protein, 23 aa, synthetic construct
EIVLTQSPATLSLSPGERATLSC

SEQ ID NO: 370 - protein, 15 aa, synthetic construct
WYQQKPGQAPRLLIY

SEQ ID NO: 371 - protein, 32 aa, synthetic construct; identical to SEQ ID NO: 19

SEQ ID NO: 372 - protein, 10 aa, synthetic construct
FGGGTKVEIK

SEQ ID NO: 373 - DNA, 69 bp, synthetic construct; identical to SEQ ID NO: 21
  source 1..69: mol_type = other DNA

SEQ ID NO: 374 - DNA, 45 bp, synthetic construct; identical to SEQ ID NO: 22
  source 1..45: mol_type = other DNA

SEQ ID NO: 375 - DNA, 96 bp, synthetic construct; identical to SEQ ID NO: 23
  source 1..96: mol_type = other DNA

SEQ ID NO: 376 - DNA, 30 bp, synthetic construct; identical to SEQ ID NO: 24
  source 1..30: mol_type = other DNA

SEQ ID NO: 377 - protein, 11 aa, synthetic construct
RASQSVSSYLA

SEQ ID NO: 378 - protein, 7 aa, synthetic construct
DASNRAT

SEQ ID NO: 379 - protein, 9 aa, synthetic construct
QQLASWPPT

SEQ ID NO: 380 - DNA, 33 bp, synthetic construct; identical to SEQ ID NO: 28
  source 1..33: mol_type = other DNA

SEQ ID NO: 381 - DNA, 21 bp, synthetic construct
  source 1..21: mol_type = other DNA
gatgcatccaacagggccact

SEQ ID NO: 382 - DNA, 27 bp, synthetic construct
  source 1..27: mol_type = other DNA
cagcagctcgcctcctggcctcctact

SEQ ID NO: 383 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQLASWPPTFGGGTKVEIK

SEQ ID NO: 384 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagctcgcctcctggcctcctacttttggcggagggaccaaggttgagatcaaa

SEQ ID NO: 385 - protein, 26 aa, synthetic construct
QVQLVQSGAEVKKPGSSVKVSCKASG

SEQ ID NO: 386 - protein, 14 aa, synthetic construct
WVRQAPGQGLEWMG

SEQ ID NO: 387 - protein, 30 aa, synthetic construct; identical to SEQ ID NO: 3

SEQ ID NO: 388 - protein, 11 aa, synthetic construct
WGQGTMVTVSS

SEQ ID NO: 389 - DNA, 78 bp, synthetic construct; identical to SEQ ID NO: 5
  source 1..78: mol_type = other DNA

SEQ ID NO: 390 - DNA, 42 bp, synthetic construct; identical to SEQ ID NO: 6
  source 1..42: mol_type = other DNA

SEQ ID NO: 391 - DNA, 90 bp, synthetic construct; identical to SEQ ID NO: 7
  source 1..90: mol_type = other DNA

SEQ ID NO: 392 - DNA, 33 bp, synthetic construct; identical to SEQ ID NO: 8
  source 1..33: mol_type = other DNA

SEQ ID NO: 393 - protein, 9 aa, synthetic construct
GTFSSYQIS

SEQ ID NO: 394 - protein, 17 aa, synthetic construct
GIIPIFGAAYYAQKFQG

SEQ ID NO: 395 - protein, 16 aa, synthetic construct
AREARVAGTRPRGFDI

SEQ ID NO: 396 - DNA, 27 bp, synthetic construct
  source 1..27: mol_type = other DNA
ggcaccttcagctcctatcaaatcagc

SEQ ID NO: 397 - DNA, 51 bp, synthetic construct
  source 1..51: mol_type = other DNA
gggatcatccctatcttcggtgccgcatactacgcacagaagttccagggc

SEQ ID NO: 398 - DNA, 48 bp, synthetic construct; identical to SEQ ID NO: 14
  source 1..48: mol_type = other DNA

SEQ ID NO: 399 - protein, 123 aa, synthetic construct
QVQLVQSGAEVKKPGSSVKVSCKASGGTFSSYQISWVRQAPGQGLEWMGGIIPIFGAAYYAQKFQGRVTITADESTSTAYMELSSLRSEDTAVYYCAREARVAGTRPRGFDIWGQGTMVTVSS

SEQ ID NO: 400 - protein, 369 aa, synthetic construct
CAGGTGCAGCTGGTGCAGTCTGGGGCTGAGGTGAAGAAGCCTGGGTCCTCGGTGAAGGTCTCCTGCAAGGCTTCTGGAGGCACCTTCAGCTCCTATCAAATCAGCTGGGTGCGACAGGCCCCTGGACAAGGGCTTGAGTGGATGGGAGGGATCATCCCTATCTTCGGTGCCGCATACTACGCACAGAAGTTCCAGGGCAGAGTCACGATTACCGCGGACGAATCCACGAGCACAGCCTACATGGAGCTGAGCAGCCTGAGATCTGAGGACACGGCGGTGTACTACTGCGCCAGAGAGGCTAGAGTAGCAGGAACACGACCACGTGGATTCGACATATGGGGTCAGGGTACAATGGTCACCGTCTCCTCA

SEQ ID NO: 401 - protein, 23 aa, synthetic construct
EIVLTQSPATLSLSPGERATLSC

SEQ ID NO: 402 - protein, 15 aa, synthetic construct
WYQQKPGQAPRLLIY

SEQ ID NO: 403 - protein, 32 aa, synthetic construct; identical to SEQ ID NO: 19

SEQ ID NO: 404 - protein, 10 aa, synthetic construct
FGGGTKVEIK

SEQ ID NO: 405 - DNA, 69 bp, synthetic construct; identical to SEQ ID NO: 21
  source 1..69: mol_type = other DNA

SEQ ID NO: 406 - DNA, 45 bp, synthetic construct; identical to SEQ ID NO: 22
  source 1..45: mol_type = other DNA

SEQ ID NO: 407 - DNA, 96 bp, synthetic construct; identical to SEQ ID NO: 23
  source 1..96: mol_type = other DNA

SEQ ID NO: 408 - DNA, 30 bp, synthetic construct; identical to SEQ ID NO: 24
  source 1..30: mol_type = other DNA

SEQ ID NO: 409 - protein, 11 aa, synthetic construct
RASQSVSSYLA

SEQ ID NO: 410 - protein, 7 aa, synthetic construct
DASNRAT

SEQ ID NO: 411 - protein, 9 aa, synthetic construct
QQLNNFPPT

SEQ ID NO: 412 - DNA, 33 bp, synthetic construct; identical to SEQ ID NO: 28
  source 1..33: mol_type = other DNA

SEQ ID NO: 413 - DNA, 21 bp, synthetic construct
  source 1..21: mol_type = other DNA
gatgcatccaacagggccact

SEQ ID NO: 414 - DNA, 27 bp, synthetic construct
  source 1..27: mol_type = other DNA
cagcagctcaacaatttccctcctact

SEQ ID NO: 415 - protein, 107 aa, synthetic construct; identical to SEQ ID NO: 31

SEQ ID NO: 416 - DNA, 321 bp, synthetic construct; identical to SEQ ID NO: 32
  source 1..321: mol_type = other DNA

SEQ ID NO: 417 - protein, 26 aa, synthetic construct
QVQLVQSGAEVKKPGSSVKVSCKASG

SEQ ID NO: 418 - protein, 14 aa, synthetic construct
WVRQAPGQGLEWMG

SEQ ID NO: 419 - protein, 30 aa, synthetic construct; identical to SEQ ID NO: 3

SEQ ID NO: 420 - protein, 11 aa, synthetic construct
WGQGTMVTVSS

SEQ ID NO: 421 - DNA, 78 bp, synthetic construct; identical to SEQ ID NO: 5
  source 1..78: mol_type = other DNA

SEQ ID NO: 422 - DNA, 42 bp, synthetic construct; identical to SEQ ID NO: 6
  source 1..42: mol_type = other DNA

SEQ ID NO: 423 - DNA, 90 bp, synthetic construct; identical to SEQ ID NO: 7
  source 1..90: mol_type = other DNA

SEQ ID NO: 424 - DNA, 33 bp, synthetic construct; identical to SEQ ID NO: 8
  source 1..33: mol_type = other DNA

SEQ ID NO: 425 - protein, 9 aa, synthetic construct
GTFSNYAIS

SEQ ID NO: 426 - protein, 17 aa, synthetic construct
GIIPIFGTAYYAQKFQG

SEQ ID NO: 427 - protein, 16 aa, synthetic construct
AREARVAGTRPRGFDI

SEQ ID NO: 428 - DNA, 27 bp, synthetic construct
  source 1..27: mol_type = other DNA
ggcaccttcagcaactatgctatcagc

SEQ ID NO: 429 - DNA, 51 bp, synthetic construct
  source 1..51: mol_type = other DNA
gggatcatccctatcttcggtacagcatactacgcacagaagttccagggc

SEQ ID NO: 430 - DNA, 48 bp, synthetic construct; identical to SEQ ID NO: 14
  source 1..48: mol_type = other DNA

SEQ ID NO: 431 - protein, 123 aa, synthetic construct
QVQLVQSGAEVKKPGSSVKVSCKASGGTFSNYAISWVRQAPGQGLEWMGGIIPIFGTAYYAQKFQGRVTITADESTSTAYMELSSLRSEDTAVYYCAREARVAGTRPRGFDIWGQGTMVTVSS

SEQ ID NO: 432 - protein, 369 aa, synthetic construct
CAGGTGCAGCTGGTGCAGTCTGGGGCTGAGGTGAAGAAGCCTGGGTCCTCGGTGAAGGTCTCCTGCAAGGCTTCTGGAGGCACCTTCAGCAACTATGCTATCAGCTGGGTGCGACAGGCCCCTGGACAAGGGCTTGAGTGGATGGGAGGGATCATCCCTATCTTCGGTACAGCATACTACGCACAGAAGTTCCAGGGCAGAGTCACGATTACCGCGGACGAATCCACGAGCACAGCCTACATGGAGCTGAGCAGCCTGAGATCTGAGGACACGGCGGTGTACTACTGCGCCAGAGAGGCTAGAGTAGCAGGAACACGACCACGTGGATTCGACATATGGGGTCAGGGTACAATGGTCACCGTCTCCTCA

SEQ ID NO: 433 - protein, 23 aa, synthetic construct
EIVLTQSPATLSLSPGERATLSC

SEQ ID NO: 434 - protein, 15 aa, synthetic construct
WYQQKPGQAPRLLIY

SEQ ID NO: 435 - protein, 32 aa, synthetic construct; identical to SEQ ID NO: 19

SEQ ID NO: 436 - protein, 10 aa, synthetic construct
FGGGTKVEIK

SEQ ID NO: 437 - DNA, 69 bp, synthetic construct; identical to SEQ ID NO: 21
  source 1..69: mol_type = other DNA

SEQ ID NO: 438 - DNA, 45 bp, synthetic construct; identical to SEQ ID NO: 22
  source 1..45: mol_type = other DNA

SEQ ID NO: 439 - DNA, 96 bp, synthetic construct; identical to SEQ ID NO: 23
  source 1..96: mol_type = other DNA

SEQ ID NO: 440 - DNA, 30 bp, synthetic construct; identical to SEQ ID NO: 24
  source 1..30: mol_type = other DNA

SEQ ID NO: 441 - protein, 11 aa, synthetic construct
RASQSVSSYLA

SEQ ID NO: 442 - protein, 7 aa, synthetic construct
DASNRAT

SEQ ID NO: 443 - protein, 9 aa, synthetic construct
QQLNNFPPT

SEQ ID NO: 444 - DNA, 33 bp, synthetic construct; identical to SEQ ID NO: 28
  source 1..33: mol_type = other DNA

SEQ ID NO: 445 - DNA, 21 bp, synthetic construct
  source 1..21: mol_type = other DNA
gatgcatccaacagggccact

SEQ ID NO: 446 - DNA, 27 bp, synthetic construct
  source 1..27: mol_type = other DNA
cagcagctcaacaatttccctcctact

SEQ ID NO: 447 - protein, 107 aa, synthetic construct; identical to SEQ ID NO: 31

SEQ ID NO: 448 - DNA, 321 bp, synthetic construct; identical to SEQ ID NO: 32
  source 1..321: mol_type = other DNA

SEQ ID NO: 449 - protein, 26 aa, synthetic construct
EVQLLESGGGLVQPGGSLRLSCAASG

SEQ ID NO: 450 - protein, 14 aa, synthetic construct
WVRQAPGKGLEWVS

SEQ ID NO: 451 - protein, 30 aa, synthetic construct; identical to SEQ ID NO: 291

SEQ ID NO: 452 - protein, 11 aa, synthetic construct
WGRGTLVTVSS

SEQ ID NO: 453 - DNA, 78 bp, synthetic construct; identical to SEQ ID NO: 293
  source 1..78: mol_type = other DNA

SEQ ID NO: 454 - DNA, 42 bp, synthetic construct; identical to SEQ ID NO: 294
  source 1..42: mol_type = other DNA

SEQ ID NO: 455 - DNA, 90 bp, synthetic construct; identical to SEQ ID NO: 295
  source 1..90: mol_type = other DNA

SEQ ID NO: 456 - DNA, 33 bp, synthetic construct; identical to SEQ ID NO: 296
  source 1..33: mol_type = other DNA

SEQ ID NO: 457 - protein, 9 aa, synthetic construct
FTFNNYPMS

SEQ ID NO: 458 - protein, 17 aa, synthetic construct
AISGSGSATFYADSVKG

SEQ ID NO: 459 - protein, 15 aa, synthetic construct
AKLGTTVSGGHYFDL

SEQ ID NO: 460 - DNA, 27 bp, synthetic construct
  source 1..27: mol_type = other DNA
ttcacctttaacaactatccaatgagc

SEQ ID NO: 461 - DNA, 51 bp, synthetic construct
  source 1..51: mol_type = other DNA
gctattagtggtagtggtagcgcaacattctacgcagactccgtgaagggc

SEQ ID NO: 462 - DNA, 45 bp, synthetic construct
  source 1..45: mol_type = other DNA
gccaagttgggaaccacagtaagcggtggtcactacttcgaccta

SEQ ID NO: 463 - protein, 122 aa, synthetic construct
EVQLLESGGGLVQPGGSLRLSCAASGFTFNNYPMSWVRQAPGKGLEWVSAISGSGSATFYADSVKGRFTISRDNSKNTLYLQMNSLRAEDTAVYYCAKLGTTVSGGHYFDLWGRGTLVTVSS

SEQ ID NO: 464 - protein, 366 aa, synthetic construct
GAGGTGCAGCTGTTGGAGTCTGGGGGAGGCTTGGTACAGCCTGGGGGGTCCCTGAGACTCTCCTGTGCAGCCTCTGGATTCACCTTTAACAACTATCCAATGAGCTGGGTCCGCCAGGCTCCAGGGAAGGGGCTGGAGTGGGTCTCAGCTATTAGTGGTAGTGGTAGCGCAACATTCTACGCAGACTCCGTGAAGGGCCGGTTCACCATCTCCAGAGACAATTCCAAGAACACGCTGTATCTGCAAATGAACAGCCTGAGAGCCGAGGACACGGCGGTGTACTACTGCGCCAAGTTGGGAACCACAGTAAGCGGTGGTCACTACTTCGACCTATGGGGGAGAGGTACCTTGGTCACCGTCTCCTCA

SEQ ID NO: 465 - protein, 23 aa, synthetic construct
EIVLTQSPATLSLSPGERATLSC

SEQ ID NO: 466 - protein, 15 aa, synthetic construct
WYQQKPGQAPRLLIY

SEQ ID NO: 467 - protein, 32 aa, synthetic construct; identical to SEQ ID NO: 19

SEQ ID NO: 468 - protein, 10 aa, synthetic construct
FGGGTKVEIK

SEQ ID NO: 469 - DNA, 69 bp, synthetic construct; identical to SEQ ID NO: 21
  source 1..69: mol_type = other DNA

SEQ ID NO: 470 - DNA, 45 bp, synthetic construct; identical to SEQ ID NO: 22
  source 1..45: mol_type = other DNA

SEQ ID NO: 471 - DNA, 96 bp, synthetic construct; identical to SEQ ID NO: 23
  source 1..96: mol_type = other DNA

SEQ ID NO: 472 - DNA, 30 bp, synthetic construct; identical to SEQ ID NO: 24
  source 1..30: mol_type = other DNA

SEQ ID NO: 473 - protein, 11 aa, synthetic construct
RASQSVSSYLA

SEQ ID NO: 474 - protein, 7 aa, synthetic construct
DASKRAT

SEQ ID NO: 475 - protein, 9 aa, synthetic construct
QQDSNHPLT

SEQ ID NO: 476 - DNA, 33 bp, synthetic construct; identical to SEQ ID NO: 28
  source 1..33: mol_type = other DNA

SEQ ID NO: 477 - DNA, 21 bp, synthetic construct
  source 1..21: mol_type = other DNA
gatgcatccaaaagggccact

SEQ ID NO: 478 - DNA, 27 bp, synthetic construct
  source 1..27: mol_type = other DNA
cagcaggacagtaaccaccctctcact

SEQ ID NO: 479 - protein, 107 aa, synthetic construct; identical to SEQ ID NO: 319

SEQ ID NO: 480 - DNA, 321 bp, synthetic construct; identical to SEQ ID NO: 320
  source 1..321: mol_type = other DNA

SEQ ID NO: 481 - protein, 26 aa, synthetic construct
EVQLLESGGGLVQPGGSLRLSCAASG

SEQ ID NO: 482 - protein, 14 aa, synthetic construct
WVRQAPGKGLEWVS

SEQ ID NO: 483 - protein, 30 aa, synthetic construct; identical to SEQ ID NO: 291

SEQ ID NO: 484 - protein, 11 aa, synthetic construct
WGRGTLVTVSS

SEQ ID NO: 485 - DNA, 78 bp, synthetic construct; identical to SEQ ID NO: 293
  source 1..78: mol_type = other DNA

SEQ ID NO: 486 - DNA, 42 bp, synthetic construct; identical to SEQ ID NO: 294
  source 1..42: mol_type = other DNA

SEQ ID NO: 487 - DNA, 90 bp, synthetic construct; identical to SEQ ID NO: 295
  source 1..90: mol_type = other DNA

SEQ ID NO: 488 - DNA, 33 bp, synthetic construct; identical to SEQ ID NO: 296
  source 1..33: mol_type = other DNA

SEQ ID NO: 489 - protein, 9 aa, synthetic construct
FTFSTSPMS

SEQ ID NO: 490 - protein, 17 aa, synthetic construct
AISGAGASTFYADSVKG

SEQ ID NO: 491 - protein, 15 aa, synthetic construct
AKLGTTVSGGHYFDL

SEQ ID NO: 492 - DNA, 27 bp, synthetic construct
  source 1..27: mol_type = other DNA
ttcacctttagcacaagcccaatgagc

SEQ ID NO: 493 - DNA, 51 bp, synthetic construct
  source 1..51: mol_type = other DNA
gctattagtggtgcaggtgcaagcacattctacgcagactccgtgaagggc

SEQ ID NO: 494 - DNA, 45 bp, synthetic construct; identical to SEQ ID NO: 302
  source 1..45: mol_type = other DNA

SEQ ID NO: 495 - protein, 122 aa, synthetic construct
EVQLLESGGGLVQPGGSLRLSCAASGFTFSTSPMSWVRQAPGKGLEWVSAISGAGASTFYADSVKGRFTISRDNSKNTLYLQMNSLRAEDTAVYYCAKLGTTVSGGHYFDLWGRGTLVTVSS

SEQ ID NO: 496 - DNA, 366 bp, synthetic construct
  source 1..366: mol_type = other DNA
gaggtgcagctgttggagtctgggggaggcttggtacagcctggggggtccctgagactctcctgtgcagcctctggattcacctttagcacaagcccaatgagctgggtccgccaggctccagggaaggggctggagtgggtctcagctattagtggtgcaggtgcaagcacattctacgcagactccgtgaagggccggttcaccatctccagagacaattccaagaacacgctgtatctgcaaatgaacagcctgagagccgaggacacggcggtgtactactgcgccaagttaggaaccacagtaagcggtggtcactacttcgacctatgggggagaggtaccttggtcaccgtctcctca

SEQ ID NO: 497 - protein, 23 aa, synthetic construct
EIVLTQSPATLSLSPGERATLSC

SEQ ID NO: 498 - protein, 15 aa, synthetic construct
WYQQKPGQAPRLLIY

SEQ ID NO: 499 - protein, 32 aa, synthetic construct; identical to SEQ ID NO: 19

SEQ ID NO: 500 - protein, 10 aa, synthetic construct
FGGGTKVEIK

SEQ ID NO: 501 - DNA, 69 bp, synthetic construct; identical to SEQ ID NO: 21
  source 1..69: mol_type = other DNA

SEQ ID NO: 502 - DNA, 45 bp, synthetic construct; identical to SEQ ID NO: 22
  source 1..45: mol_type = other DNA

SEQ ID NO: 503 - DNA, 96 bp, synthetic construct; identical to SEQ ID NO: 23
  source 1..96: mol_type = other DNA

SEQ ID NO: 504 - protein, 30 aa, synthetic construct
TTTGGCGGAGGGACCAAGGTTGAGATCAAA

SEQ ID NO: 505 - protein, 11 aa, synthetic construct
RASQSVSSYLA

SEQ ID NO: 506 - protein, 7 aa, synthetic construct
DASKRAT

SEQ ID NO: 507 - protein, 9 aa, synthetic construct
QQDSNHPLT

SEQ ID NO: 508 - DNA, 33 bp, synthetic construct; identical to SEQ ID NO: 28
  source 1..33: mol_type = other DNA

SEQ ID NO: 509 - DNA, 21 bp, synthetic construct
  source 1..21: mol_type = other DNA
gatgcatccaaaagggccact

SEQ ID NO: 510 - DNA, 27 bp, synthetic construct
  source 1..27: mol_type = other DNA
cagcaggacagtaaccaccctctcact

SEQ ID NO: 511 - protein, 107 aa, synthetic construct; identical to SEQ ID NO: 319

SEQ ID NO: 512 - DNA, 321 bp, synthetic construct; identical to SEQ ID NO: 320
  source 1..321: mol_type = other DNA

SEQ ID NO: 513 - protein, 26 aa, synthetic construct
EVQLVESGGGLVKPGGSLRLSCAASG

SEQ ID NO: 514 - protein, 14 aa, synthetic construct
WVRQAPGKGLEWVS

SEQ ID NO: 515 - protein, 30 aa, synthetic construct
RFTISRDNAKNSLYLQMNSLRAEDTAVYYC

SEQ ID NO: 516 - protein, 11 aa, synthetic construct
WGRGTLVTVSS

SEQ ID NO: 517 - DNA, 78 bp, synthetic construct; identical to SEQ ID NO: 293
  source 1..78: mol_type = other DNA

SEQ ID NO: 518 - protein, 42 aa, synthetic construct
TGGGTCCGCCAGGCTCCAGGGAAGGGGCTGGAGTGGGTCTCA

SEQ ID NO: 519 - DNA, 90 bp, synthetic construct; identical to SEQ ID NO: 295
  source 1..90: mol_type = other DNA

SEQ ID NO: 520 - DNA, 33 bp, synthetic construct; identical to SEQ ID NO: 296
  source 1..33: mol_type = other DNA

SEQ ID NO: 521 - protein, 9 aa, synthetic construct
FTFSSYPMV

SEQ ID NO: 522 - protein, 17 aa, synthetic construct
SISISASYIDYADSVKG

SEQ ID NO: 523 - protein, 15 aa, synthetic construct
AKLGTTVSGGHYFDL

SEQ ID NO: 524 - DNA, 27 bp, synthetic construct
  source 1..27: mol_type = other DNA
ttcaccttcagtagctatcccatggtc

SEQ ID NO: 525 - DNA, 51 bp, synthetic construct
  source 1..51: mol_type = other DNA
tccattagtatcagtgcaagttacatagactacgcagactcagtgaagggc

SEQ ID NO: 526 - DNA, 45 bp, synthetic construct; identical to SEQ ID NO: 302
  source 1..45: mol_type = other DNA

SEQ ID NO: 527 - protein, 122 aa, synthetic construct
EVQLVESGGGLVKPGGSLRLSCAASGFTFSSYPMVWVRQAPGKGLEWVSSISISASYIDYADSVKGRFTISRDNAKNSLYLQMNSLRAEDTAVYYCAKLGTTVSGGHYFDLWGRGTLVTVSS

SEQ ID NO: 528 - protein, 366 aa, synthetic construct
GAGGTGCAGCTGGTGGAGTCTGGGGGAGGCCTGGTCAAGCCTGGGGGGTCCCTGAGACTCTCCTGTGCAGCCTCTGGATTCACCTTCAGTAGCTATCCCATGGTCTGGGTCCGCCAGGCTCCAGGGAAGGGGCTGGAGTGGGTCTCATCCATTAGTATCAGTGCAAGTTACATAGACTACGCAGACTCAGTGAAGGGCCGATTCACCATCTCCAGAGACAACGCCAAGAACTCACTGTATCTGCAAATGAACAGCCTGAGAGCCGAGGACACGGCGGTGTACTACTGCGCCAAGTTAGGAACCACAGTAAGCGGTGGTCACTACTTCGACCTATGGGGGAGAGGTACCTTGGTCACCGTCTCCTCA

SEQ ID NO: 529 - protein, 23 aa, synthetic construct
EIVLTQSPATLSLSPGERATLSC

SEQ ID NO: 530 - protein, 15 aa, synthetic construct
WYQQKPGQAPRLLIY

SEQ ID NO: 531 - protein, 32 aa, synthetic construct; identical to SEQ ID NO: 19

SEQ ID NO: 532 - protein, 10 aa, synthetic construct
FGGGTKVEIK

SEQ ID NO: 533 - DNA, 69 bp, synthetic construct; identical to SEQ ID NO: 21
  source 1..69: mol_type = other DNA

SEQ ID NO: 534 - DNA, 45 bp, synthetic construct; identical to SEQ ID NO: 22
  source 1..45: mol_type = other DNA

SEQ ID NO: 535 - DNA, 96 bp, synthetic construct; identical to SEQ ID NO: 23
  source 1..96: mol_type = other DNA

SEQ ID NO: 536 - DNA, 30 bp, synthetic construct; identical to SEQ ID NO: 24
  source 1..30: mol_type = other DNA

SEQ ID NO: 537 - protein, 11 aa, synthetic construct
RASQSVSSYLA

SEQ ID NO: 538 - protein, 7 aa, synthetic construct
DASKRAT

SEQ ID NO: 539 - protein, 9 aa, synthetic construct
QQDSNHPLT

SEQ ID NO: 540 - DNA, 33 bp, synthetic construct; identical to SEQ ID NO: 28
  source 1..33: mol_type = other DNA

SEQ ID NO: 541 - DNA, 21 bp, synthetic construct
  source 1..21: mol_type = other DNA
gatgcatccaaaagggccact

SEQ ID NO: 542 - DNA, 27 bp, synthetic construct
  source 1..27: mol_type = other DNA
cagcaggacagtaaccaccctctcact

SEQ ID NO: 543 - skipped

SEQ ID NO: 544 - DNA, 321 bp, synthetic construct; identical to SEQ ID NO: 320
  source 1..321: mol_type = other DNA

SEQ ID NO: 545 - protein, 26 aa, synthetic construct
EVQLLESGGGLVQPGGSLRLSCAASG

SEQ ID NO: 546 - protein, 14 aa, synthetic construct
WVRQAPGKGLEWVS

SEQ ID NO: 547 - protein, 30 aa, synthetic construct; identical to SEQ ID NO: 291

SEQ ID NO: 548 - protein, 11 aa, synthetic construct
WGRGTLVTVSS

SEQ ID NO: 549 - DNA, 78 bp, synthetic construct; identical to SEQ ID NO: 293
  source 1..78: mol_type = other DNA

SEQ ID NO: 550 - DNA, 42 bp, synthetic construct; identical to SEQ ID NO: 294
  source 1..42: mol_type = other DNA

SEQ ID NO: 551 - DNA, 90 bp, synthetic construct; identical to SEQ ID NO: 295
  source 1..90: mol_type = other DNA

SEQ ID NO: 552 - DNA, 33 bp, synthetic construct; identical to SEQ ID NO: 296
  source 1..33: mol_type = other DNA

SEQ ID NO: 553 - protein, 9 aa, synthetic construct
FTFSSSAMV

SEQ ID NO: 554 - protein, 17 aa, synthetic construct
DISASGGYTSYADSVKG

SEQ ID NO: 555 - protein, 15 aa, synthetic construct
AKLGTTVSGGHYFDL

SEQ ID NO: 556 - DNA, 27 bp, synthetic construct
  source 1..27: mol_type = other DNA
ttcacctttagcagctccgccatggtc

SEQ ID NO: 557 - DNA, 51 bp, synthetic construct
  source 1..51: mol_type = other DNA
gacattagtgcaagtggtggttacacatcctacgcagactccgtgaagggc

SEQ ID NO: 558 - DNA, 45 bp, synthetic construct; identical to SEQ ID NO: 302
  source 1..45: mol_type = other DNA

SEQ ID NO: 559 - protein, 122 aa, synthetic construct
EVQLLESGGGLVQPGGSLRLSCAASGFTFSSSAMVWVRQAPGKGLEWVSDISASGGYTSYADSVKGRFTISRDNSKNTLYLQMNSLRAEDTAVYYCAKLGTTVSGGHYFDLWGRGTLVTVSS

SEQ ID NO: 560 - protein, 366 aa, synthetic construct
GAGGTGCAGCTGTTGGAGTCTGGGGGAGGCTTGGTACAGCCTGGGGGGTCCCTGAGACTCTCCTGTGCAGCCTCTGGATTCACCTTTAGCAGCTCCGCCATGGTCTGGGTCCGCCAGGCTCCAGGGAAGGGGCTGGAGTGGGTCTCAGACATTAGTGCAAGTGGTGGTTACACATCCTACGCAGACTCCGTGAAGGGCCGGTTCACCATCTCCAGAGACAATTCCAAGAACACGCTGTATCTGCAAATGAACAGCCTGAGAGCCGAGGACACGGCGGTGTACTACTGCGCCAAGTTAGGAACCACAGTAAGCGGTGGTCACTACTTCGACCTATGGGGGAGAGGTACCTTGGTCACCGTCTCCTCA

SEQ ID NO: 561 - protein, 23 aa, synthetic construct
EIVLTQSPATLSLSPGERATLSC

SEQ ID NO: 562 - protein, 15 aa, synthetic construct
WYQQKPGQAPRLLIY

SEQ ID NO: 563 - protein, 32 aa, synthetic construct; identical to SEQ ID NO: 19

SEQ ID NO: 564 - protein, 10 aa, synthetic construct
FGGGTKVEIK

SEQ ID NO: 565 - DNA, 92 bp, synthetic construct
  source 1..92: mol_type = other DNA
gaaattgtgttgacacagtctccgaaattgtgttgacacagtctccagccaccctgtctttgtctccaggggaaagagccaccctctcctgc

SEQ ID NO: 566 - DNA, 45 bp, synthetic construct; identical to SEQ ID NO: 22
  source 1..45: mol_type = other DNA

SEQ ID NO: 567 - DNA, 96 bp, synthetic construct; identical to SEQ ID NO: 23
  source 1..96: mol_type = other DNA

SEQ ID NO: 568 - DNA, 30 bp, synthetic construct; identical to SEQ ID NO: 24
  source 1..30: mol_type = other DNA

SEQ ID NO: 569 - protein, 11 aa, synthetic construct
RASQSVSSYLA

SEQ ID NO: 570 - protein, 7 aa, synthetic construct
DASKRAT

SEQ ID NO: 571 - protein, 9 aa, synthetic construct
QQDSNHPLT

SEQ ID NO: 572 - DNA, 33 bp, synthetic construct; identical to SEQ ID NO: 28
  source 1..33: mol_type = other DNA

SEQ ID NO: 573 - DNA, 21 bp, synthetic construct
  source 1..21: mol_type = other DNA
gatgcatccaaaagggccact

SEQ ID NO: 574 - DNA, 27 bp, synthetic construct
  source 1..27: mol_type = other DNA
cagcaggacagtaaccaccctctcact

SEQ ID NO: 575 - protein, 107 aa, synthetic construct; identical to SEQ ID NO: 319

SEQ ID NO: 576 - DNA, 321 bp, synthetic construct; identical to SEQ ID NO: 320
  source 1..321: mol_type = other DNA

(19) United States
(12) Patent Application Publication
(10) Pub. No.: US 2025/0381255 A1
(43) Pub. Date: Dec. 18, 2025

(54) ANTI-ASGR1 POLYPEPTIDES AND METHODS OF USE FOR IMMUNE TOLERANCE

(71) Applicant: Anokion SA, Ecublens (CH)

(72) Inventors: Stephan Kontos, Belmont, CA (US); Gregory Paul Conley, Arlington, MA (US); Dina Sami Wassaf, Burlington, MA (US); Rebecca llene Goldstein, Boston, MA (US); Julie Michelle Silverman, Somerville, MA (US); Nels Peter Nielson, II, Hopkinton, NH (US); Noel Thomas Pauli, Lebanon, NH (US)

(21) Appl. No.: 18/834,346

(22) PCT Filed: Feb. 16, 2023
(86) PCT No.: PCT/US2023/062761

§ 371 Date: Jul. 30, 2024

Related U.S. Application Data
(60) Provisional application No. 63/268,183, filed on Feb. 17, 2022.
(60) Provisional application No. 63/268,190, filed on Feb. 17, 2022.
(60) Provisional application No. 63/483,456, filed on Feb. 6, 2023.
(60) Provisional application No. 63/483,466, filed on Feb. 6, 2023.

(51) Int. Cl. A61K 39/00; A61K 39/35; A61K 39/385; A61P 3/10; A61P 37/04; C07K 16/28

(52) U.S. Cl. CPC A61K 39/0008; A61K 39/001; A61K 39/35; A61K 39/385; A61P 3/10; A61P 37/04; C07K 16/2851; A61K 2039/6056; C07K 2317/52; C07K 2317/565

(57) ABSTRACT

Disclosed herein are binding polypeptides that bind to asialoglycoprotein receptor 1 (ASGR1), and methods of use thereof for inducing immune tolerance against antigens of interest, for example, for treating, ameliorating, inhibiting, or preventing disease or disorders associated with unwanted immune response against the antigens of interest, such as autoimmune diseases.

### CROSS-REFERENCE TO RELATED APPLICATIONS

This application is the United States National Phase Application under 35 U.S.C. § 371 of International Patent Application No. PCT/US2023/062761, filed Feb. 16, 2023, which claims priority to U.S. Provisional Patent Application No. 63/268,183, filed Feb. 17, 2022, U.S. Provisional Patent Application No. 63/268,190, filed Feb. 17, 2022, U.S. Provisional Patent Application No. 63/483,456, filed Feb. 6, 2023, and U.S. Provisional Patent Application No. 63/483,466, filed Feb. 6, 2023, the entire contents of each of which is incorporated by reference herein.

### REFERENCE TO SEQUENCE LISTING

The present application is being filed along with a Sequence Listing in electronic format. The Sequence Listing is provided as an XML file entitled 95611_01900_ST26.xml which was created and last modified on Jul. 2, 2025, which is 648,605 bytes in size. The information in the electronic Sequence Listing is hereby incorporated by reference in its entirety.

### FIELD

Aspects of the present disclosure relate to binding polypeptides that bind to ASGR1 and methods of use thereof for inducing immune tolerance against desired antigens, such as for the prevention or treatment of a disease.

### BACKGROUND

The liver is involved in a variety of tolerogenic processes. For example, it plays a role in the development of immune tolerance to non-self-antigens absorbed into the blood draining from the gut or to newly formed antigens resulting from hepatic metabolic activities. Targeting the liver with therapeutic compositions could be beneficial for induction of antigen-specific immune tolerance.

### SUMMARY

Antigens such as non-self-antigens absorbed into the blood draining from the gut or newly formed antigens resulting from hepatic metabolic activities fail to induce an immune response in healthy individuals. Antigen-specific immune tolerance and cross-tolerance induction towards CD4+ and CD8+ T cells, respectively, has been attributed to various cell types in the liver including hepatocytes and liver sinusoidal endothelial cells (LSECs). Hepatocytes are the predominant cell type that make up the liver parenchyma, and they can process and present antigens on MHC-I and MHC-II to signal to CD8+ and CD4+ T cells, respectively. LSECs efficiently scavenge, process and present soluble antigens from the bloodstream on MHC-I and MHC-II to circulating lymphocytes, typically resulting in the induction of CD4+ regulatory T cells or anergic CD8+ T cells.

Disclosed herein are asialoglycoprotein receptor 1 (ASGR1) binding polypeptides. In some embodiments, the ASGR1 binding polypeptides comprise a heavy chain variable region. In some embodiments, the heavy chain variable region comprises one or more of an HCDR1, HCDR2, and HCDR3. In some embodiments, the ASGR1 binding polypeptides comprise or further comprise a light chain variable region. In some embodiments, the light chain variable region comprises one or more of an LCDR1, LCDR2, and LCDR3.

Also disclosed herein are tolerogenic compounds comprising an ASGR1 binding polypeptide conjugated or fused to an antigen to which tolerance is desired. The ASGR1 binding polypeptide and antigen may be conjugated or fused with a linker. The ASGR1 binding polypeptide and antigen may be chemically conjugated with a chemical conjugation linker. The ASGR1 binding polypeptide and antigen may be recombinantly fused.

Also disclosed herein are compositions comprising any one of the tolerogenic compounds disclosed herein and a pharmaceutically acceptable excipient.

Also disclosed herein are methods of inducing tolerance to an antigen to which a subject is capable of developing an unwanted immune response. In some embodiments, the methods comprise administering any one of the tolerogenic compounds or compositions disclosed herein to the subject.

Also disclosed herein are the tolerogenic compounds or compositions disclosed herein for use in inducing tolerance to an antigen to which a subject is capable of developing an unwanted immune response in the subject, or for use in the manufacture of a medicament.

In several embodiments, there is provided an asialoglycoprotein receptor 1 (ASGR1) binding polypeptide comprising a heavy chain variable (VH) region comprising a first heavy chain complementarity determining region (HCDR1), a second heavy chain complementarity determining region (HCDR2), and a third heavy chain complementarity determining region (HCDR3). In several embodiments, the HCDR1 comprises the sequence of SEQ ID NO: 9. In several embodiments, the HCDR2 comprises the sequence of SEQ ID NO: 10. In several embodiments, the HCDR3 comprises the sequence of SEQ ID NO: 11. In several embodiments, the HCDR1 comprises the sequence of SEQ ID NO: 9, the HCDR2 comprises the sequence of SEQ ID NO: 10, and the HCDR3 comprises the sequence of SEQ ID NO: 11. In several embodiments, the VH comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 15.

In several embodiments, the ASGR1 binding polypeptide further comprises a light chain variable region (VL) comprising a first light chain complementarity determining region (LCDR1), a second light chain complementarity determining region (LCDR2), and a third light chain complementarity determining region (LCDR3). In several embodiments, the LCDR1 comprises the sequence of SEQ ID NO: 25. In several embodiments, the LCDR2 comprises the sequence of SEQ ID NO: 26. In several embodiments, the LCDR3 comprises the sequence of SEQ ID NO: 27. In several embodiments, the LCDR1 comprises the sequence of SEQ ID NO: 25, the LCDR2 comprises the sequence of SEQ ID NO: 26, and the LCDR3 comprises the sequence of SEQ ID NO: 27. In several embodiments, the VL comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 31.

In several embodiments, the heavy chain variable region comprises the sequence of SEQ ID NO: 15 and the light chain variable region comprises the sequence of SEQ ID NO: 31.

In several embodiments, the ASGR1 binding polypeptide is an antibody, an Fab′ fragment, an F(ab′)2 fragment, a domain antibody (dAb), or an scFv. In several embodiments, the ASGR1 binding polypeptide comprises an Fc domain, optionally wherein the Fc domain is silenced.

In several embodiments, there is provided a polynucleotide encoding an ASGR1 binding polypeptide, the polynucleotide comprising one or more of the sequences of SEQ ID NO: 12-14, or sequences having at least 95% identity thereto. In several embodiments, the polynucleotide comprises the sequence of SEQ ID NO: 16. In several embodiments, the polynucleotide comprises one or more of the sequences of SEQ ID NO: 25-27. In several embodiments, the polynucleotide comprises the sequence of SEQ ID NO: 32. In several embodiments, the polynucleotide further comprises the sequence of SEQ ID NO: 16.

In several embodiments, there is provided a tolerogenic compound comprising an ASGR1 binding polypeptide as disclosed herein, wherein the ASGR1 binding polypeptide is conjugated or fused to an antigen to which tolerance is desired. In several embodiments, the ASGR1 binding polypeptide and the antigen are conjugated or fused with a linker, optionally wherein the linker is a polypeptide linker or a chemical conjugation linker. In several embodiments, the linker is a cleavable linker. In several embodiments, the linker comprises glycine and/or serine, optionally wherein the linker comprises the sequence of SEQ ID NO: 37 or a sequence having at least about 80%, 85%, 90%, or 95% identity thereto. In several embodiments, the antigen is conjugated or fused to the N-terminus or C-terminus of the ASGR1 binding polypeptide.

In several embodiments, the antigen comprises a food antigen. In several embodiments, the food antigen is associated with celiac disease. In several embodiments, the antigen comprises a sequence having at least 80%, 85%, 90%, 95%, 96%, 97%, 98%, 99%, or 100% sequence identity to SEQ ID NO: 41. In several embodiments, the antigen comprises SEQ ID NO: 41. In several embodiments, the food antigen is selected from conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), a-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1IS globulin (Sin a 2), lipid transfer protein (Sin a 3), profilin (Sin a 4), profilin (Api g 4), high molecular weight glycoprotein (Api g 5), tropomyosin (Pen a 1), arginine kinase (Pen m 2), tropomyosin fast isoform, high molecular weight glutenin, low molecular weight glutenin, alpha-, gamma- and omega-gliadin, hordein, secalin, avenin, pathogenesis-related protein from strawberries (Fra a 1), profilin (Mus a 1), a portion of any of said antigens, and a mimetic of any of said antigens. In several embodiments, the food antigen is selected from the group consisting of high molecular weight glutenin, low molecular weight glutenin, alpha-, gamma- and omega-gliadin, hordein, secalin, avenin, a portion of any of said antigens, and a mimetic of any of said antigens. In several embodiments, the antigen comprises a sequence having at least 80%, 85%, 90%, 95%, 96%, 97%, 98%, 99%, or 100% sequence identity to any one of SEQ ID NO: 40-54, or a fragment thereof.

In several embodiments, the antigen comprises an autoantigen. In several embodiments, the autoantigen is selected from thyroglobulin, thyroperoxidase, thyroid-stimulating hormone receptor, glutamic acid decarboxylase (GAD), 21OH hydroxylase, 17OH hydroxylase, H+/K+ ATPase, intrinsic factor, transglutaminase, tyrosinase, tyrosinase-related protein-2, myelin basic protein, proteolipid protein, desmogleins, acetylcholine receptor, 2-oxoacid dehydrogenase complexes, insulin, proinsulin, preproinsulin, insulinoma-associated protein 2 (IA-2), insulinoma-associated protein 213 (IA-213), ICA69, ICA12 (SOX-13), carboxypeptidase H, imogen 38, GLIMA 38, chromogranin-A, HSP-60, carboxypeptidase E, peripherin, glucose transporter 2, hepatocarcinoma-intestine-pancreas/pancreatic associated protein, S100ß, glial fibrillary acidic protein, regenerating gene II, pancreatic duodenal homeobox 1, dystrophia myotonica protein kinase (DMPK), islet-specific glucose-6-phosphatase catalytic subunit-related protein, SST G-protein coupled receptors 1-5, myeloperoxidase (MPO), proteinase-3/myeloblastin, and a portion of any of said antigens, and a mimetic of any of said antigens.

In several embodiments, the antigen comprises an antigen associated with an autoimmune disease. In several embodiments, the autoimmune disease is selected from the group consisting of multiple sclerosis, type 1 diabetes, rheumatoid arthritis, vitiligo, uveitis, pemphigus vulgaris, neuromyelitis optica, Goodpasture's Disease, Parkinson's disease, myasthenia gravis, celiac disease, primary biliary cholangitis, Sjogren's syndrome, autoimmune hepatitis, myocarditis, inflammatory cardiomyopathy, and anti-neutrophil cytoplasmic antibody-associated vasculitis. In several embodiments, the autoimmune disease is multiple sclerosis. In several embodiments, the antigen comprises a sequence having at least 80%, 85%, 90%, 95%, 96%, 97%, 98%, 99%, or 100% sequence identity to any one of SEQ ID NO: 100, 71-99, 101-106 or 157-159, or a fragment thereof. In several embodiments, the autoimmune disease is type 1 diabetes. In several embodiments, the antigen comprises a sequence having at least 80%, 85%, 90%, 95%, 96%, 97%, 98%, 99%, or 100% sequence identity to any one of SEQ ID NO: 55-70 or 153-156, or a fragment thereof.

In several embodiments, there is provided a tolerogenic compound comprising an ASGR1 binding polypeptide conjugated or fused to an antigen to which tolerance is desired, wherein the ASGR1 binding polypeptide comprises a heavy chain variable region comprising one, two or all three of HCDR1, HCDR2, and HCDR3; wherein the HCDR1 comprises the sequence of SEQ ID NO: 9; wherein the HCDR2 comprises the sequence of SEQ ID NO: 10; and/or wherein the HCDR3 comprises the sequence of SEQ ID NO: 11. In several embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 15. In several embodiments, the ASGR1 binding polypeptide further comprises a light chain variable region comprising one, two or all three of LCDR1, LCDR2, and LCDR3; wherein the LCDR1 comprises the sequence of SEQ ID NO: 25; wherein the LCDR2 comprises the sequence of SEQ ID NO: 26; and/or wherein the LCDR3 comprises the sequence of SEQ ID NO: 27. In several embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 31. In several embodiments, the heavy chain variable region comprises the sequence of SEQ ID NO: 15 and the light chain variable region comprises the sequence of SEQ ID NO: 31. In several embodiments, the ASGR1 binding polypeptide is an antibody, an Fab′ fragment, an F (ab′) 2 fragment, a domain antibody (dAb), or an scFv. In several embodiments, the ASGR1 binding polypeptide comprises an Fc domain, optionally wherein the Fc domain is silenced. In several embodiments, the antigen is a polypeptide.

In several embodiments, the ASGR1 binding polypeptide and the antigen are conjugated or fused with a linker, optionally wherein the linker is a polypeptide linker or a chemical conjugation linker. In several embodiments, the linker is a cleavable linker. In several embodiments, the linker comprises glycine and/or serine, optionally wherein the linker comprises the sequence of SEQ ID NO: 37. In several embodiments, the antigen is conjugated or fused to the N-terminus or C-terminus of the ASGR1 binding polypeptide. In several embodiments, the ASGR1 binding polypeptide comprises an Fc domain, optionally wherein the Fc domain is silenced, and the antigen is conjugated or fused to the Fc domain, optionally wherein the antigen is conjugated or fused to the C-terminus of the Fc domain.

In several embodiments, the antigen comprises a food antigen. In several embodiments, the food antigen is selected from conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), a-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1IS globulin (Sin a 2), lipid transfer protein (Sin a 3), profilin (Sin a 4), profilin (Api g 4), high molecular weight glycoprotein (Api g 5), tropomyosin (Pen a 1), arginine kinase (Pen m 2), tropomyosin fast isoform, high molecular weight glutenin, low molecular weight glutenin, alpha-, gamma- and omega-gliadin, hordein, secalin, avenin, pathogenesis-related protein from strawberries (Fra a 1), profilin (Mus a 1), a portion of any of said antigens, and a mimetic of any of said antigens. In several embodiments, the food antigen is selected from the group consisting of high molecular weight glutenin, low molecular weight glutenin, alpha-, gamma- and omega-gliadin, hordein, secalin, avenin, a portion of any of said antigens, and a mimetic of any of said antigens. In several embodiments, the food antigen is associated with celiac disease. In several embodiments, the antigen comprises a sequence having at least 80%, 85%, 90%, 95%, 96%, 97%, 98%, 99%, or 100% sequence identity to any one of SEQ ID NO: 40-54, or a fragment thereof.

In several embodiments, the antigen comprises an autoantigen. In several embodiments, the the autoantigen is selected from thyroglobulin, thyroperoxidase, thyroid-stimulating hormone receptor, glutamic acid decarboxylase (GAD), 21OH hydroxylase, 17OH hydroxylase, H+/K+ ATPase, intrinsic factor, transglutaminase, tyrosinase, tyrosinase-related protein-2, myelin basic protein, proteolipid protein, desmogleins, acetylcholine receptor, 2-oxoacid dehydrogenase complexes, insulin, proinsulin, preproinsulin, insulinoma-associated protein 2 (IA-2), insulinoma-associated protein 213 (IA-213), ICA69, ICA12 (SOX-13), carboxypeptidase H, imogen 38, GLIMA 38, chromogranin-A, HSP-60, carboxypeptidase E, peripherin, glucose transporter 2, hepatocarcinoma-intestine-pancreas/pancreatic associated protein, S100ß, glial fibrillary acidic protein, regenerating gene II, pancreatic duodenal homeobox 1, dystrophia myotonica protein kinase (DMPK), islet-specific glucose-6-phosphatase catalytic subunit-related protein, SST G-protein coupled receptors 1-5, myeloperoxidase (MPO), proteinase-3/myeloblastin, and a portion of any of said antigens, and a mimetic of any of said antigens.

In several embodiments, the antigen comprises an antigen associated with an autoimmune disease. In several embodiments, the autoimmune disease is selected from the group consisting of type 1 diabetes, multiple sclerosis, rheumatoid arthritis, vitiligo, uveitis, pemphigus vulgaris, neuromyelitis optica, Goodpasture's Disease, Parkinson's disease, myasthenia gravis, celiac disease, primary biliary cholangitis, Sjogren's syndrome, autoimmune hepatitis, myocarditis, inflammatory cardiomyopathy, and anti-neutrophil cytoplasmic antibody-associated vasculitis. In several embodiments, the autoimmune disease is type 1 diabetes. In several embodiments, the antigen comprises a sequence having at least 80%, 85%, 90%, 95%, 96%, 97%, 98%, 99%, or 100% sequence identity to any one of SEQ ID NO: 55-70 or 153-156, or a fragment thereof. In several embodiments, the autoimmune disease is multiple sclerosis. In several embodiments, the the antigen comprises a sequence having at least 80%, 85%, 90%, 95%, 96%, 97%, 98%, 99%, or 100% sequence identity to any one of SEQ ID NO: 71-106 or 157-159, or a fragment thereof. In several embodiments, the autoimmune disease is rheumatoid arthritis, wherein the antigen comprises a sequence having at least 80%, 85%, 90%, 95%, 96%, 97%, 98%, 99%, or 100% sequence identity to any one of SEQ ID NO: 107-116, 160-239 or 272-288, or a fragment thereof, Sjogren's syndrome, wherein the antigen comprises a sequence having at least 80%, 85%, 90%, 95%, 96%, 97%, 98%, 99%, or 100% sequence identity to any one of SEQ ID NO: 117-118 or 240-245, or a fragment thereof, rheumatic heart disease, autoimmune myocarditis, viral myocarditis, or inflammatory cardiomyopathy, wherein the antigen comprises a sequence having at least 80%, 85%, 90%, 95%, 96%, 97%, 98%, 99%, or 100% sequence identity to any one of SEQ ID NO: 119 or 251-270, or a fragment thereof, Parkinson's disease, wherein the antigen comprises a sequence having at least 80%, 85%, 90%, 95%, 96%, 97%, 98%, 99%, or 100% sequence identity to any one of SEQ ID NO: 120 or 247-250, or a fragment thereof, anti-neutrophil cytoplasmic antibody-associated vasculitis (ANCA-vasculitis), wherein the antigen comprises a sequence having at least 80%, 85%, 90%, 95%, 96%, 97%, 98%, 99%, or 100% sequence identity to any one of SEQ ID NO: 121-129, or a fragment thereof, primary biliary cholangitis, wherein the antigen comprises a sequence having at least 80%, 85%, 90%, 95%, 96%, 97%, 98%, 99%, or 100% sequence identity to any one of SEQ ID NO: 130-132 or 271, or a fragment thereof; or autoimmune hepatitis, wherein the antigen comprises a sequence having at least 80%, 85%, 90%, 95%, 96%, 97%, 98%, 99%, or 100% sequence identity to any one of SEQ ID NO: 133-140, or a fragment thereof.

In several embodiments, the antigen comprises an alloantigen. In several embodiments, the alloantigen is selected from the group consisting of subunits of the MHC class I and MHC class II haplotype proteins and their complexes with the antigens they present, and minor blood group antigens RhCE, Kell, Kidd, Duffy, Diego, and MNSs.

Also provided is a composition comprising the tolerogenic compound according to the present disclosure and a pharmaceutically acceptable excipient.

Provided herein are methods of inducing tolerance to an antigen to which a subject is capable of developing an unwanted immune response, comprising administering the tolerogenic compound or compostions provided herein to the subject. In several embodiments, the compound or the composition is administered prior to the subject being exposed to the antigen, after the subject has been exposed to the antigen, or both. In several embodiments, the the unwanted immune response is associated with an allergy towards a food, animal, plant, or environmental allergen, an autoimmune disease, a therapeutic agent, or graft-vs-host disease.

In several embodiments, there is provided a tolerogenic compound or composition as disclosed herein for use in inducing tolerance in a subject to an antigen to which the subject is capable of developing an unwanted immune response. In several embodiments, the unwanted immune response is associated with an allergy towards a food, animal, plant, or environmental allergen, an autoimmune disease, a therapeutic agent, or graft-vs-host disease. In several embodiments, the tolerogenic compound or the composition is for use in the manufacture of a medicament. In several embodiments, the composition is for use in the induction of immune tolerance in a subject in need thereof.

Also provided for herein is a method of inducing tolerance to an antigen for which tolerance is desired, the method comprising administering to a subject a compound comprising: (i) an asialoglycoprotein receptor 1 (ASGR1) binding polypeptide comprising a heavy chain variable region comprising an HCDR1, HCDR2, and HCDR3; wherein the HCDR1 comprises a sequence having at least 90% identity to SEQ ID NO: 9; wherein the HCDR2 comprises a sequence having at least 90% identity to SEQ ID NO: 10; and wherein the HCDR3 comprises a sequence having at least 90% identity to SEQ ID NO: 11; and (ii) an antigen to which tolerance is desired, wherein the ASGR1 binding polypeptide is conjugated or fused to the antigen to which tolerance is desired.

In several embodiments, the ASGR1 binding polypeptide further comprises a light chain variable region comprising an LCDR1, LCDR2, and LCDR3, wherein the LCDR1 comprises a sequence having at least 90% identity to SEQ ID NO: 25, wherein the LCDR2 comprises a sequence having at least 90% identity to SEQ ID NO: 26, and wherein the LCDR3 comprises a sequence having at least 90% identity to SEQ ID NO: 27.

In several embodiments, the antigen to which tolerance is desired is associated with Celiac Disease. In several embodiments, the antigen comprises a sequence having at least 80%, 85%, 90%, 95%, 96%, 97%, 98%, 99%, or 100% sequence identity to any one of SEQ ID NO: 41, 40, 43-54, or a fragment thereof. In several embodiments, the antigen to which tolerance is desired is associated with MS. In several embodiments, the antigen comprises a sequence having at least 80%, 85%, 90%, 95%, 96%, 97%, 98%, 99%, or 100% sequence identity to any one of SEQ ID NO: 100, 71-99, 101-106 or 157-159, or a fragment thereof. In several embodiments, the antigen to which tolerance is desired is associated with type 1 diabetes. In several embodiments, the antigen comprises a sequence having at least 80%, 85%, 90%, 95%, 96%, 97%, 98%, 99%, or 100% sequence identity to any one of SEQ ID NO: 55-70 or 153-156, or a fragment thereof.

In several embodiments, the administering is via an intravenous route.

Ise there is provided a method of delivering an antigen to liver tissue of a subject, the method comprising: conjugating or fusing an antigen to an asialoglycoprotein receptor 1 (ASGR1) binding polypeptide, thereby generating an ASGR1 binding polypeptide-antigen complex; and causing the ASGR1 binding polypeptide-antigen complex to be contacted with liver tissue of a subject in situ, wherein the contacting allows the ASGR1 binding polypeptide to bind to ASGR1 on the liver tissue, thereby delivering the antigen to liver tissue.

In additional embodiments, there is provided a method of delivering an antigen to liver tissue of a subject, the method comprising, causing an asialoglycoprotein receptor 1 (ASGR1) binding polypeptide, conjugated of fused to an antigen to contact liver tissue of a subject in situ, thereby allowing the ASGR1 binding polypeptide to bind to ASGR1 on the liver tissue, wherein the ASGR1 binding polypeptide comprises a heavy chain variable region comprising an HCDR1, HCDR2, and HCDR3, wherein the HCDR1 comprises a sequence having at least 90% identity to SEQ ID NO: 9, wherein the HCDR2 comprises a sequence having at least 90% identity to SEQ ID NO: 10; and wherein the HCDR3 comprises a sequence having at least 90% identity to SEQ ID NO: 11; and wherein the ASGR1 binding polypeptide comprises a light chain variable region comprising an LCDR1, LCDR2, and LCDR3, wherein the LCDR1 comprises a sequence having at least 90% identity to SEQ ID NO: 25; wherein the LCDR2 comprises a sequence having at least 90% identity to SEQ ID NO: 26; and wherein the LCDR3 comprises a sequence having at least 90% identity to SEQ ID NO: 27. In several embodiments, the delivery of the antigen to the liver tissue of the subject in situ induces tolerance in the subject to the antigen. In several embodiments, the method is for treatment of an autoimmune disease. In several embodiments, the method is for treatment of an allergy.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 297; wherein the HCDR2 comprises the sequence of SEQ ID NO: 298; wherein the HCDR3 comprises the sequence of SEQ ID NO: 299; wherein the LCDR1 comprises the sequence of SEQ ID NO: 313; wherein the LCDR2 comprises the sequence of SEQ ID NO: 314; and wherein the LCDR3 comprises the sequence of SEQ ID NO: 315. In several embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 303. In several embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 319. In several embodiments, the heavy chain variable region comprises the sequence of SEQ ID NO: 303 and the light chain variable region comprises the sequence of SEQ ID NO: 319.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 329; wherein the HCDR2 comprises the sequence of SEQ ID NO: 330; wherein the HCDR3 comprises the sequence of SEQ ID NO: 331; wherein the LCDR1 comprises the sequence of SEQ ID NO: 345; wherein the LCDR2 comprises the sequence of SEQ ID NO: 346; and wherein the LCDR3 comprises the sequence of SEQ ID NO: 347.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 361; wherein the HCDR2 comprises the sequence of SEQ ID NO: 362; wherein the HCDR3 comprises the sequence of SEQ ID NO: 363; wherein the LCDR1 comprises the sequence of SEQ ID NO: 377; wherein the LCDR2 comprises the sequence of SEQ ID NO: 378; and wherein the LCDR3 comprises the sequence of SEQ ID NO: 379.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 393; wherein the HCDR2 comprises the sequence of SEQ ID NO: 394; wherein the HCDR3 comprises the sequence of SEQ ID NO: 395; wherein the LCDR1 comprises the sequence of SEQ ID NO: 409; wherein the LCDR2 comprises the sequence of SEQ ID NO: 410; and wherein the LCDR3 comprises the sequence of SEQ ID NO: 411.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 425; wherein the HCDR2 comprises the sequence of SEQ ID NO: 426; wherein the HCDR3 comprises the sequence of SEQ ID NO: 427; wherein the LCDR1 comprises the sequence of SEQ ID NO: 441; wherein the LCDR2 comprises the sequence of SEQ ID NO: 442; and wherein the LCDR3 comprises the sequence of SEQ ID NO: 443.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 457; wherein the HCDR2 comprises the sequence of SEQ ID NO: 458; wherein the HCDR3 comprises the sequence of SEQ ID NO: 459; wherein the LCDR1 comprises the sequence of SEQ ID NO: 473; wherein the LCDR2 comprises the sequence of SEQ ID NO: 474; and wherein the LCDR3 comprises the sequence of SEQ ID NO: 475.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 489; wherein the HCDR2 comprises the sequence of SEQ ID NO: 490; wherein the HCDR3 comprises the sequence of SEQ ID NO: 491; wherein the LCDR1 comprises the sequence of SEQ ID NO: 505; wherein the LCDR2 comprises the sequence of SEQ ID NO: 506; and wherein the LCDR3 comprises the sequence of SEQ ID NO: 507.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 521; wherein the HCDR2 comprises the sequence of SEQ ID NO: 522; wherein the HCDR3 comprises the sequence of SEQ ID NO: 523; wherein the LCDR1 comprises the sequence of SEQ ID NO: 537; wherein the LCDR2 comprises the sequence of SEQ ID NO: 538; and wherein the LCDR3 comprises the sequence of SEQ ID NO: 539.

In several embodiments, there is provided an asialoglycoprotein receptor 1 (ASGR1) binding polypeptide comprising a heavy chain variable region comprising an HCDR1, HCDR2, HCDR3 and a light chain variable region comprising an LCDR1, LCDR2 and LCDR3; wherein the HCDR1 comprises the sequence of SEQ ID NO: 553; wherein the HCDR2 comprises the sequence of SEQ ID NO: 554; wherein the HCDR3 comprises the sequence of SEQ ID NO: 555; wherein the LCDR1 comprises the sequence of SEQ ID NO: 569; wherein the LCDR2 comprises the sequence of SEQ ID NO: 570; and wherein the LCDR3 comprises the sequence of SEQ ID NO: 571.

In several embodiments, there is provided for herein an asialoglycoprotein receptor 1 (ASGR1) binding polypeptide comprising a heavy chain variable region comprising an HCDR1, HCDR2, and HCDR3, wherein the HCDR1 comprises the sequence of SEQ ID NO: 9; wherein the HCDR2 comprises the sequence of SEQ ID NO: 10; and wherein the HCDR3 comprises the sequence of SEQ ID NO: 11. In several embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 15.

In several embodiments, the ASGR1 binding polypeptide further comprises a light chain variable region comprising an LCDR1, LCDR2, and LCDR3, wherein the LCDR1 comprises the sequence of SEQ ID NO: 25, wherein the LCDR2 comprises the sequence of SEQ ID NO: 26; and wherein the LCDR3 comprises the sequence of SEQ ID NO: 27. In several embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 31. In several embodiments, the heavy chain variable region comprises the sequence of SEQ ID NO: 15 and the light chain variable region comprises the sequence of SEQ ID NO: 31.

Depending on the embodiment, the ASGR1 binding polypeptide is optionally an antibody, an Fab′ fragment, an F(ab′)2 fragment, a domain antibody (dAb), or an scFv. In several embodiments, the ASGR1 binding polypeptide comprises an Fc domain, optionally wherein the Fc domain is silenced.

There is also provided for herein a polynucleotide encoding for an ASGR1 binding polypeptide, the polynucleotide comprising one or more of the sequences of SEQ ID NO: 12-14 or 25-27. In several embodiments, the polynucleotide comprises the sequence of SEQ ID NO: 16 or 32, or both. In several embodiments, the polynucleotide encodes an ASGR1 binding polypeptide according to the disclosure herein.

In several embodiments, there is also provided tolerogenic compound comprising an ASGR1 binding polypeptide conjugated or fused to an antigen to which tolerance is desired. In several embodiments, he ASGR1 binding polypeptide comprises a heavy chain variable region comprising an HCDR1, HCDR2, and HCDR3, wherein the HCDR1 comprises the sequence of SEQ ID NO: 9, wherein the HCDR2 comprises the sequence of SEQ ID NO: 10, and wherein the HCDR3 comprises the sequence of SEQ ID NO: 11, In several embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 15.

In several embodiments, the tolerogenic compounds provided for herein also comprise a light chain variable region comprising an LCDR1, LCDR2, and LCDR3, wherein the LCDR1 comprises the sequence of SEQ ID NO: 25, wherein the LCDR2 comprises the sequence of SEQ ID NO: 26, and wherein the LCDR3 comprises the sequence of SEQ ID NO: 27. In several embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 31. In several embodiments, the wherein the heavy chain variable region comprises the sequence of SEQ ID NO: 15 and the light chain variable region comprises the sequence of SEQ ID NO: 31.

In several embodiments of the tolerogenic compounds provided for herein, the ASGR1 binding polypeptide is an antibody, an Fab′ fragment, an F(ab′)2 fragment, a domain antibody (dAb), or an scFv. In several embodiments, of the tolerogenic compounds provided for herein, the ASGR1 binding polypeptide comprises an Fc domain, optionally wherein the Fc domain is silenced.

In several embodiments of the tolerogenic compounds provided for herein, the antigen is a polypeptide. In several embodiments, the ASGR1 binding polypeptide and the antigen are conjugated or fused with a linker, optionally wherein the linker is a polypeptide linker or a chemical conjugation linker. In some embodiments, the linker is a cleavable linker. In several embodiments, the linker comprises glycine and/or serine, optionally wherein the linker comprises the sequence of SEQ ID NO: 37. In several embodiments, the antigen is conjugated or fused to the N-terminus or C-terminus of the ASGR1 binding polypeptide. In some embodiments of the tolerogenic compounds provided for herein, the ASGR1 binding polypeptide comprises an Fc domain, optionally wherein the Fc domain is silenced, and the antigen is conjugated or fused to the Fc domain, optionally wherein the antigen is conjugated or fused to the C-terminus of the Fc domain.

In several embodiments of the tolerogenic compounds provided for herein, the antigen comprises a food antigen. In several embodiments, the food antigen is selected from conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), a-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1IS globulin (Sin a 2), lipid transfer protein (Sin a 3), profilin (Sin a 4), profilin (Api g 4), high molecular weight glycoprotein (Api g 5), tropomyosin (Pen a 1), arginine kinase (Pen m 2), tropomyosin fast isoform, high molecular weight glutenin, low molecular weight glutenin, alpha-, gamma- and omega-gliadin, hordein, secalin, avenin, pathogenesis-related protein from strawberries (Fra a 1), profilin (Mus a 1), a portion of any of said antigens, and a mimetic of any of said antigens.

In several embodiments, the food antigen is selected from the group consisting of high molecular weight glutenin, low molecular weight glutenin, alpha-, gamma- and omega-gliadin, hordein, secalin, avenin, a portion of any of said antigens, and a mimetic of any of said antigens.

In several embodiments, the food antigen is associated with celiac disease. In several embodiments, the antigen comprises a sequence having at least 80%, 85%, 90%, 95%, 96%, 97%, 98%, 99%, or 100% sequence identity to any one of SEQ ID NO: 40-54, or a fragment thereof.

In several embodiments of the tolerogenic compounds provided for herein, the antigen comprises an autoantigen. In several embodiments, the autoantigen is selected from thyroglobulin, thyroperoxidase, thyroid-stimulating hormone receptor, glutamic acid decarboxylase (GAD), 21OH hydroxylase, 17OH hydroxylase, H+/K+ ATPase, intrinsic factor, transglutaminase, tyrosinase, tyrosinase-related protein-2, myelin basic protein, proteolipid protein, desmogleins, acetylcholine receptor, 2-oxoacid dehydrogenase complexes, insulin, proinsulin, preproinsulin, insulinoma-associated protein 2 (IA-2), insulinoma-associated protein 213 (IA-213), ICA69, ICA12 (SOX-13), carboxypeptidase H, imogen 38, GLIMA 38, chromogranin-A, HSP-60, carboxypeptidase E, peripherin, glucose transporter 2, hepatocarcinoma-intestine-pancreas/pancreatic associated protein, S100ß, glial fibrillary acidic protein, regenerating gene II, pancreatic duodenal homeobox 1, dystrophia myotonica protein kinase (DMPK), islet-specific glucose-6-phosphatase catalytic subunit-related protein, SST G-protein coupled receptors 1-5, myeloperoxidase (MPO), proteinase-3/myeloblastin, and a portion of any of said antigens, and a mimetic of any of said antigens.

In several embodiments of the tolerogenic compounds provided for herein, the antigen comprises an antigen associated with an autoimmune disease. In several embodiments, the autoimmune disease is selected from the group consisting of type 1 diabetes, multiple sclerosis, rheumatoid arthritis, vitiligo, uveitis, pemphigus vulgaris, neuromyelitis optica, Goodpasture's Disease, Parkinson's disease, myasthenia gravis, celiac disease, primary biliary cholangitis, Sjogren's syndrome, autoimmune hepatitis, myocarditis, inflammatory cardiomyopathy, and anti-neutrophil cytoplasmic antibody-associated vasculitis.

In several embodiments of the tolerogenic compounds provided for herein, the autoimmune disease is type 1 diabetes. In several embodiments, the antigen comprises a sequence having at least 80%, 85%, 90%, 95%, 96%, 97%, 98%, 99%, or 100% sequence identity to any one of SEQ ID NO: 55-70 or 153-156, or a fragment thereof.

In several embodiments of the tolerogenic compounds provided for herein, the autoimmune disease is multiple sclerosis. In several embodiments, the antigen comprises a sequence having at least 80%, 85%, 90%, 95%, 96%, 97%, 98%, 99%, or 100% sequence identity to any one of SEQ ID NO: 71-106 or 157-159, or a fragment thereof.

In several embodiments of the tolerogenic compounds provided for herein, the autoimmune disease is rheumatoid arthritis. In several embodiments, the antigen comprises a sequence having at least 80%, 85%, 90%, 95%, 96%, 97%, 98%, 99%, or 100% sequence identity to any one of SEQ ID NO: 107-116, 160-239 or 272-288, or a fragment thereof.

In several embodiments of the tolerogenic compounds provided for herein, the autoimmune disease is Sjogren's syndrome. In several embodiments, the antigen comprises a sequence having at least 80%, 85%, 90%, 95%, 96%, 97%, 98%, 99%, or 100% sequence identity to any one of SEQ ID NO: 117-118 or 240-245, or a fragment thereof.

In several embodiments of the tolerogenic compounds provided for herein, the autoimmune disease is rheumatic heart disease, autoimmune myocarditis, viral myocarditis, or inflammatory cardiomyopathy. In several embodiments, the antigen comprises a sequence having at least 80%, 85%, 90%, 95%, 96%, 97%, 98%, 99%, or 100% sequence identity to any one of SEQ ID NO: 119 or 251-270, or a fragment thereof.

In several embodiments of the tolerogenic compounds provided for herein, the autoimmune disease is Parkinson's disease. In several embodiments, the antigen comprises a sequence having at least 80%, 85%, 90%, 95%, 96%, 97%, 98%, 99%, or 100% sequence identity to any one of SEQ ID NO: 120 or 247-250, or a fragment thereof.

In several embodiments of the tolerogenic compounds provided for herein, the autoimmune disease is anti-neutrophil cytoplasmic antibody-associated vasculitis (ANCA-vasculitis). In several embodiments, the antigen comprises a sequence having at least 80%, 85%, 90%, 95%, 96%, 97%, 98%, 99%, or 100% sequence identity to any one of SEQ ID NO: 121-129, or a fragment thereof.

In several embodiments of the tolerogenic compounds provided for herein, the autoimmune disease is primary biliary cholangitis. In several embodiments, the antigen comprises a sequence having at least 80%, 85%, 90%, 95%, 96%, 97%, 98%, 99%, or 100% sequence identity to any one of SEQ ID NO: 130-132 or 271, or a fragment thereof.

In several embodiments of the tolerogenic compounds provided for herein, the autoimmune disease is autoimmune hepatitis. In several embodiments, the antigen comprises a sequence having at least 80%, 85%, 90%, 95%, 96%, 97%, 98%, 99%, or 100% sequence identity to any one of SEQ ID NO: 133-140, or a fragment thereof.

In several embodiments of the tolerogenic compounds provided for herein, the antigen comprises an alloantigen. In several embodiments, the alloantigen is selected from the group consisting of subunits of the MHC class I and MHC class II haplotype proteins and their complexes with the antigens they present, and minor blood group antigens RhCE, Kell, Kidd, Duffy, Diego, and MNSs.

There is also provided for herein, in several embodiments, a composition comprising a tolerogenic compound according to the present disclosure and a pharmaceutically acceptable excipient.

Also provided for herein are methods of inducing tolerance to an antigen to which a subject is capable of developing an unwanted immune response, comprising administering a tolerogenic compound according to the present disclosure or a composition of according to the present disclosure to the subject.

In several embodiments, the tolerogenic compound or the composition is administered prior to the subject being exposed to the antigen, after the subject has been exposed to the antigen, or both.

In several embodiments, the unwanted immune response is associated with an allergy towards a food, animal, plant, or environmental allergen, an autoimmune disease, a therapeutic agent, or graft-vs-host disease.

Also provided for herein are the tolerogenic compounds of the present disclosure and/or the compositions of the present disclosure for use in inducing tolerance in a subject to an antigen to which the subject is capable of developing an unwanted immune response. In several embodiments, the unwanted immune response is associated with an allergy towards a food, animal, plant, or environmental allergen, an autoimmune disease, a therapeutic agent, or graft-vs-host disease.

Also provided for herein are the tolerogenic compounds of the present disclosure and/or the compositions of the present disclosure for use in the manufacture of a medicament. Additionally, provided for herein is a compound of the present disclosure and/or a composition of the present disclosure for use in the induction of immune tolerance in a subject in need thereof.

In several embodiments, there is provided a method of inducing tolerance to an antigen for which tolerance is desired, the method comprising administering to a subject a compound comprising (i) an asialoglycoprotein receptor 1 (ASGR1) binding polypeptide comprising a heavy chain variable region comprising an HCDR1, HCDR2, and HCDR3, wherein the HCDR1 comprises a sequence having at least 90% identity to SEQ ID NO: 9, wherein the HCDR2 comprises a sequence having at least 90% identity to SEQ ID NO: 10, and wherein the HCDR3 comprises a sequence having at least 90% identity to SEQ ID NO: 11; and (ii) an antigen to which tolerance is desired, wherein the ASGR1 binding polypeptide is conjugated or fused to the antigen to which tolerance is desired.

In several embodiments, the ASGR1 binding polypeptide further comprises a light chain variable region comprising an LCDR1, LCDR2, and LCDR3, wherein the LCDR1 comprises a sequence having at least 90% identity to SEQ ID NO: 25, wherein the LCDR2 comprises a sequence having at least 90% identity to SEQ ID NO: 26, and wherein the LCDR3 comprises a sequence having at least 90% identity to SEQ ID NO: 27.

In several embodiments of the methods provided, the antigen to which tolerance is desired is associated with Celiac Disease. In several embodiments, the antigen comprises a sequence having at least 80%, 85%, 90%, 95%, 96%, 97%, 98%, 99%, or 100% sequence identity to any one of SEQ ID NO: 40-54, or a fragment thereof.

In several embodiments of the methods provided, the antigen to which tolerance is desired is associated with multiple sclerosis (MS). In several embodiments, the antigen comprises a sequence having at least 80%, 85%, 90%, 95%, 96%, 97%, 98%, 99%, or 100% sequence identity to any one of SEQ ID NO: 71-106 or 157-159, or a fragment thereof.

In several embodiments of the methods provided, wherein the antigen to which tolerance is desired is associated with type 1 diabetes. In several embodiments, the antigen comprises a sequence having at least 80%, 85%, 90%, 95%, 96%, 97%, 98%, 99%, or 100% sequence identity to any one of SEQ ID NO: 55-70 or 153-156, or a fragment thereof.

In several embodiments of the methods provided, the administering is via an intravenous route.

Also provided for herein are methods of delivering an antigen to liver tissue of a subject, the method comprising conjugating or fusing an antigen to an asialoglycoprotein receptor 1 (ASGR1) binding polypeptide, thereby generating an ASGR1 binding polypeptide-antigen complex; and causing the ASGR1 binding polypeptide-antigen complex to be contacted with liver tissue of a subject in situ, wherein the contacting allows the ASGR1 binding polypeptide to bind to ASGR1 on the liver tissue, thereby delivering the antigen to liver tissue.

Additionally provided are method a of delivering an antigen to liver tissue of a subject, the method comprising causing an asialoglycoprotein receptor 1 (ASGR1) binding polypeptide, conjugated of fused to an antigen to contact liver tissue of a subject in situ, thereby allowing the ASGR1 binding polypeptide to bind to ASGR1 on the liver tissue, wherein the ASGR1 binding polypeptide comprises a heavy chain variable region comprising an HCDR1, HCDR2, and HCDR3, wherein the HCDR1 comprises a sequence having at least 90% identity to SEQ ID NO: 9, wherein the HCDR2 comprises a sequence having at least 90% identity to SEQ ID NO: 10, and wherein the HCDR3 comprises a sequence having at least 90% identity to SEQ ID NO: 11; and wherein the ASGR1 binding polypeptide comprises a light chain variable region comprising an LCDR1, LCDR2, and LCDR3, wherein the LCDR1 comprises a sequence having at least 90% identity to SEQ ID NO: 25, wherein the LCDR2 comprises a sequence having at least 90% identity to SEQ ID NO: 26, and wherein the LCDR3 comprises a sequence having at least 90% identity to SEQ ID NO: 27. In several embodiments, the delivery of the antigen to the liver tissue of the subject in situ induces tolerance in the subject to the antigen. In several embodiments, such methods are for treatment of an autoimmune disease. In several embodiments, such methods are for treatment of an allergy.

### BRIEF DESCRIPTION OF THE DRAWINGS

In addition to the features described herein, additional features and variations will be readily apparent from the following descriptions of the drawings and embodiments. It is to be understood that these drawings depict various embodiments and are not intended to be limiting in scope.

FIG. 1A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 1B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 1C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 1D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 1E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 1F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 2A depicts sequences for human asialoglycoprotein receptor 1 (ASGR1) (SEQ ID NO: 33), the extracellular domain (ECD) thereof spanning from amino acids Q62-L291 (SEQ ID NO: 34), and a His-Avi tagged variant (SEQ ID NO: 35) that may be used for antigen production and purification.

FIG. 2B depicts sequences of the p31 mimotope peptide that induces T1D pathology in BDC2.5 models, a conjugatable p31 variant with a cysteine linker to attach an ASGR1-binding polypeptide, a non-limiting example of a glycine-serine linker, and a glycine-serine linker-p31 variant that can be used for antibody fusions.

FIG. 3 depicts a survival curve showing that liver-targeted antigen protects mice from T1D in the pre-activated BDC2.5 model. NOD.SCID mice received an adoptive transfer of 3×10⁵ activated BDC2.5 T cells i.v. on day 0 and were treated on days 0 and 4 with 50 pmol/g of αASGR1-p31, 50 pmol/g p31, or saline i.v. Blood glucose was measured 2-3 times a week for 105 days. Mice were considered diabetic after two consecutive blood glucose measurements ≥250 mg/dL. The experiment was conducted 7-8 animals per group.

FIG. 4 depicts a diabetes onset survival curve of NOD.SCID mice with adoptively transferred BCD2.5 splenocytes treated with a p31 tolerogen fused with anti-ASGR1 antibody mAb-60819 (819-p31), p31 peptide only, or saline control.

FIG. 5 depicts an EAE disease severity curve of EAE model mice injected with encephalitogenic T cells and liver-targeted tolerogens of MOG10 fused to mAb-60819 (819-MOG10). 819-MOG10 was administered at either 2 pmol/g (“dose level 1”) or 10 pmol/g (“dose level 2”).

FIG. 6A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 6B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 6C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 6D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 6E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 6F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 7 depicts a diabetes onset survival curve of NOD.SCID mice with adoptively transferred BCD2.5 splenocytes treated with a p31 tolerogen fused with anti-ASGR1 antibody mAb-60856 (856-p31), p31 peptide only, or saline control.

FIG. 8A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 8B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 8C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 8D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 8E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 8F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 9A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 9B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 9C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 9D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 9E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 9F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 10 depicts a diabetes onset survival curve of NOD.SCID mice with adoptively transferred BCD2.5 splenocytes treated with a p31 tolerogen fused with anti-ASGR1 antibody mAb-60869 (869-p31), p31 peptide only, or saline control.

FIG. 11A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 11B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 11C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 11D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 11E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 11F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 12A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 12B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 12C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 12D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 12E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 12F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 13A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 13B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 13C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 13D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 13E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 13F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 14A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 14B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 14C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 14D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 14E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 14F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 15A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 15B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 15C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 15D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 15E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 15F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 16A depicts non-limiting heavy chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 16B depicts non-limiting heavy chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 16C depicts non-limiting heavy chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 16D depicts non-limiting light chain framework sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 16E depicts non-limiting light chain complementarity-determining region (CDR) sequences that may be used for the anti-ASGR1 antibodies provided herein. The provided DNA sequences encode the provided peptide sequences, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

FIG. 16F depicts non-limiting light chain variable region sequences that may be used for the anti-ASGR1 antibodies provided herein. The provided DNA sequence encodes the provided peptide sequence, but other DNA sequences that can encode the same peptide sequence by virtue of codon degeneracy are also envisioned. Also envisioned are peptide and DNA sequences that have at least 85%, 86%, 87%, 88%, 89%, 90%, 91%, 92%, 93%, 94%, 95%, 96%, 97%, 98%, 99%, or 100% identity to the sequences provided therein.

### DETAILED DESCRIPTION

Immune reactions against various antigens can be a significant source of morbidity and mortality. Immune reactions can develop in an individual that lead to adverse impacts on the health and well-being of the individual, reduced efficacy of a treatment being received by an individual, and even reactions to endogenous molecules naturally occurring or existing in the individual. While broad immune suppression is utilized in certain scenarios to address certain types of immune responses, these can lead to generalized susceptibility to infection and sickness. Thus, a more tailored approach, such as those described herein, is advantageous in that antigen-specific immune responses can be targeted. Several embodiments disclosed herein leverage the role of the liver, and its various types of cells, in the development of immune tolerance to specific antigens. For example, in several embodiments, specific antigens, immunogenic fragments thereof, and/or mimetics thereof (collectively, antigens, unless otherwise indicated as a specific type, e.g., a fragment), are linked or coupled to a molecule that is configured to target the liver (or specific cells within or associated with the liver), thereby allowing the specific antigen, immunogenic fragments thereof, and/or mimetics thereof, to be processed and the immune system to be recalibrated to reduce, ameliorate, or otherwise eliminate an immune response against that antigen (or portion of an antigen, or a plurality of antigens). For example, in several embodiments, compositions provided herein are targeted for delivery to (and for uptake by) the liver, such as hepatocytes or other cells that express scavenger receptors (e.g., asialoglycoprotein receptors (ASGPRs), etc.).

Some embodiments disclosed herein demonstrate that hepatocytes can be manipulated using synthetic constructs, such as those compositions disclosed herein, to actively induce immunologic tolerance of antigen-specific CD4+ or CD8+ T cells, for example, by presentation or cross-presentation of extracellular antigens. Considered “non-professional antigen-presenting cells,” hepatocytes are a promising cellular candidate for antigen presentation to prime tolerogenic T cell responses. Hepatocytes compose up to 80% of the total liver and are in direct contact with circulating T lymphocytes. Hepatocytes do not express immunological co-stimulatory molecules. For that reason, whether hepatocytes contribute to peripheral tolerogenesis by presenting and cross-presenting blood-borne antigens was tested. Demonstrated herein, and in accordance with several embodiments, hepatocytes can be manipulated in situ (e.g., through targeting hepatocytes with constructs according to embodiments disclosed herein) to contribute to peripheral tolerogenesis by presenting and cross-presenting antigens to T cells.

Unlike other organs, where circulating lymphocytes only extravasate and gain access to the parenchyma in the case of inflammation, the liver microvasculature has a peculiar fenestrated endothelium devoid of any basal membrane, allowing direct physical contact between circulating T lymphocytes and liver MHC+ parenchymal cells, including hepatocytes. Hepatocytes possess poor cross-presentation capacity in vitro as compared to other liver cells, especially LSECs. Nonetheless, direct antigen expression, obtained by transgenesis and/or viral vector transduction, and subsequent MHC-I-dependent antigen presentation in hepatocytes in vitro and in vivo can result in immune tolerance mainly by suboptimal activation of antigen-specific CD8+ T lymphocytes because of a lack of CD28 co-stimulation leading to clonal deletion of the T cells. The induction of CD4+CD25+FoxP3+ Treg cells also occurs upon lentiviral-mediated hepatocyte-dependent antigen presentation, indicating a possible involvement of other antigen-presenting cells (APCs) in hepatocyte-driven tolerogenic mechanisms, since hepatocytes express low levels of MHC-II to interact with CD4+ T cells.

Hepatocytes outnumber other cellular components of the liver and are in close contact with components of the blood. In some embodiments disclosed herein, hepatocytes are used to establish CD4+ and CD8+ T cell peripheral tolerance through mechanisms of extracellular antigen uptake and presentation or cross-presentation. In other embodiments, the constructs and compositions disclosed herein are used to induce tolerance through other mechanisms, alone or in conjunction with antigen presentation or cross-presentation. Hepatocytes possess lectin receptors (among others), including the asialoglycoprotein receptor (ASGPR). Apoptotic processes activate neuraminidases that desialylate glycoproteins to expose terminal N-acetylgalactosamine residues, which bind to ASGPR. Hepatocyte-dependent antigen presentation or cross-presentation (among other mechanisms induced by liver-resident immune cells following administration of the constructs disclosed herein [including the induction of regulatory T cells], and related methods) can be used in methods to induce immune tolerance via T cell deletion and/or anergy more generally. In some embodiments, hepatocytes are useful as target cells for tolerogenic prophylactic or therapeutic interventions.

Generally, the compositions provided herein comprise an antigen of interest (e.g., one to which immune tolerance is desired, including antigenic fragments of a larger molecule, or in some embodiments, a plurality of antigens/fragments thereof), a targeting moiety (e.g., a molecule, particularly a binding polypeptide or an antibody, that specifically targets or is recognized by the liver, or a cell type within the liver, or another target organ or cell, e.g., in the lymph nodes and/or spleen), where the antigen of interest and the targeting moiety are connected, such as with a linker. In some embodiments, mimetics of those antigens may be used instead of the antigen or antigen fragments.

Approaches for connecting the antigen of interest and the targeting moiety are generally known in the art. For example, when the antigen of interest and the targeting moiety are both proteins (e.g. when the targeting moiety is an antibody), the antigen of interest and the targeting moiety can be fused together in different configurations, such as the termini of either protein. In some embodiments, the antigen of interest and the targeting moiety can be recombinantly fused together in different configurations. In some embodiments, the antigen of interest and the targeting moiety can be fused using conventional recombinant cloning techniques. The antigen of interest and the targeting moiety may be connected with a linker, as generally understood in the art. In several embodiments, the linkers are advantageously designed and/or configured to release the antigen (or antigenic fragment(s) thereof or mimetic thereof) in vivo in its native, or substantially native form (e.g., the form in which it was prior to being conjugated or fused to the linker). In some embodiments, the antigenic portion of the molecule is attached to the linker via a degradable bond. Thus, in several embodiments, the antigen of interest is liberated at, in or near the liver (or other target site) and is processed and presented to the immune system in a manner that allows the immune system to recognize the native antigen (or antigenic fragment thereof or mimetic thereof) as self, and reduce or eliminate an immune response against that antigen.

In several embodiments, the antigen can be endogenous (e.g., a self-antigen or autoantigen) or exogenous (e.g., a foreign antigen), including but not limited to: a foreign alloantigen against which transplant recipients develop an unwanted immune response (e.g., graft-vs-host disease or transplant rejection), a foreign food, animal, plant or environmental antigen to which patients develop an unwanted immune (e.g., allergic or hypersensitivity) response, a therapeutic agent to which patients develop an unwanted immune response (e.g., hypersensitivity and/or reduced therapeutic activity), an autoantigen to which patients develop an unwanted immune response (e.g., autoimmune disease), or a tolerogenic portion (e.g., a fragment or an epitope) thereof. The compositions provided herein are useful for inducing tolerization to the antigen and for treating an unwanted immune response, e.g., graft-vs-host disease, transplant rejection, an immune response against a therapeutic agent, an autoimmune disease, and/or an allergy, depending on the embodiment.

Also provided are pharmaceutical compositions containing a therapeutically effective amount of a compound of the disclosure. In some embodiments, the compound is admixed with at least one pharmaceutically acceptable excipient. In another aspect, the disclosure provides methods for the treatment of an unwanted immune response, such as graft-vs-host disease, transplant rejection, response against a therapeutic agent, autoimmune disease or allergy.

### Terms

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

In several embodiments, the antigen-binding polypeptide comprises one or more complementarity determining regions (“CDR” or “CDRs”. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains.

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al, (1997) JMB 273,927-948 (“Chothia” numbering scheme); MacCallum et al, J. Mol. Biol. 262:732-745 (1996), “Antibody-antigen interactions: Contact analysis and binding site topography,” J. Mol. Biol. 262, 732-745.” (“Contact” numbering scheme); Lefranc M P et al, “IMGT unique numbering for immunoglobulin and T cell receptor variable domains and Ig superfamily V-like domains,” Dev Comp Immunol, 2003 January; 27 (I): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Martin et ah, “Modeling antibody hypervariable loops: a combined algorithm,” PNAS, 1989, 86 (23): 9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

As used herein, an “antigen” shall have its plain and ordinary meaning and shall refer to any substance that serves as a target for the receptors of an innate or adaptive immune response, such as the T cell receptor, major histocompatibility complex class I and II, B cell receptor or an antibody. In some embodiments, an antigen may originate from within the body (e.g., “self,” “auto” or “endogenous”). In additional embodiments, an antigen may originate from outside the body (“non-self,” “foreign” or “exogenous”), having entered, for example, by inhalation, ingestion, injection, or transplantation, transdermally, etc. In some embodiments, an exogenous antigen may be biochemically modified in the body. Foreign antigens include, but are not limited to, food antigens, animal antigens, plant antigens, environmental antigens, therapeutic agents, as well as antigens present in an allograft transplant.

As used herein, the term “epitope”, also known as antigenic determinant, shall have its plain and ordinary meaning and shall refer to a segment of a macromolecule (e.g. a protein), which is recognized by the immune system, such as by antibodies, B cells, major histocompatibility complex molecules, or T cells. Epitopes may be recognized by, for example, antibodies or B cells, and may include a part or segment of a macromolecule capable of binding to an antibody or antigen-binding fragment thereof. In this context, the term “binding” in particular relates to a specific binding. In the context of several embodiments of the present invention, it is preferred that the term “epitope” refers to a segment of protein or polyprotein that is recognized by the immune system. In several embodiments, the “antigen” used in the constructs disclosed herein may comprise one or more epitopes. In some embodiments where more than one epitope is included, the additional epitopes may be from the same or a different antigen.

A peptide that specifically binds a particular target is referred to as a “ligand” for that target.

“Specific binding,” as a term that is commonly used in the biological arts, refers to a molecule that binds to a target with a relatively high affinity as compared to non-target tissues, and generally involves a plurality of non-covalent interactions, such as electrostatic interactions, van der Waals interactions, hydrogen bonding, and the like. Specific binding interactions characterize antibody-antigen binding, enzyme-substrate binding, and certain protein-receptor interactions; while such molecules might bind tissues besides their specific targets from time to time, to the extent that such non-target binding is inconsequential, the high-affinity binding pair can still fall within the definition of specific binding.

As used herein, the term “conservative changes” shall have its plain and ordinary meaning and refers to changes that can generally be made to an amino acid sequence without altering activity. These changes are termed “conservative substitutions” or mutations; that is, an amino acid belonging to a grouping of amino acids having a particular size or characteristic can be substituted for another amino acid. Substitutes for an amino acid sequence can be selected from other members of the class to which the amino acid belongs. For example, the nonpolar (hydrophobic) amino acids include alanine, leucine, isoleucine, valine, proline, phenylalanine, tryptophan, methionine, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such substitutions are not expected to substantially affect apparent molecular weight as determined by polyacrylamide gel electrophoresis or isoelectric point. Conservative substitutions also include substituting optical isomers of the sequences for other optical isomers, specifically D-amino acids for L-amino acids for one or more residues of a sequence. Moreover, all of the amino acids in a sequence can undergo a D- to L-isomer substitution. Examples of conservative substitutions include, but are not limited to, Lys for Arg and vice versa to maintain a positive charge; Glu for Asp and vice versa to maintain a negative charge; Ser for Thr so that a free-OH is maintained; and GIn for Asn to maintain a free —NH₂. Yet another type of conservative substitution constitutes the case where amino acids with desired chemical reactivities are introduced to impart reactive sites for chemical conjugation reactions if the need for chemical derivatization arises. Such amino acids include but are not limited to Cys (to insert a sulfhydryl group), Lys (to insert a primary amine), Asp and Glu (to insert a carboxylic acid group), or specialized noncanonical amino acids containing ketone, azide, alkyne, alkene, and tetrazine side-chains. Conservative substitutions or additions of free —NH₂ or —SH bearing amino acids can be particularly advantageous for chemical conjugation with linkers. Moreover, point mutations, deletions, and insertions of the polypeptide sequences or corresponding nucleic acid sequences can in some cases be made without a loss of function of the polypeptide or nucleic acid fragment. Substitutions can include, e.g., 1, 2, 3, 4, 5, 6, 7, 8, 9, 10, 15, 20, 25, 30, 35, 40, 45, 50 or more residues (including any number of substitutions between those listed). A variant usable in the present invention may exhibit a total number of up to 200 (e.g., up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200, including any number in between those listed) changes in the amino acid sequence (e.g., exchanges, insertions, deletions, N-terminal truncations, and/or C-terminal truncations). In several embodiments, the number of changes is greater than 200. Additionally, in several embodiments, the variants include polypeptide sequences or corresponding nucleic acid sequences that exhibit a degree of functional equivalence with a reference (e.g., unmodified or native sequence). In several embodiments, the variants exhibit about 80%, about 85%, about 90%, about 95%, about 97%, about 98%, about 99% functional equivalence to an unmodified or native reference sequence (and any degree of functional equivalence between those listed). The amino acid residues described herein employ either the single letter amino acid designator or the three-letter abbreviation in keeping with the standard polypeptide nomenclature. All amino acid residue sequences are represented herein by formulae with left and right orientation in the conventional direction of amino-terminus to carboxy-terminus.

The term “sequence identity” is used with regard to polypeptide or nucleic acid sequence comparisons. This expression in particular refers to a percentage of sequence that is the same, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or nucleic acid after aligning the sequences and introducing gaps, if necessary, to achieve the maximum percent sequence identity, and without considering any conservative substitutions as part of the sequence identity. Alignment for the purpose of determining percent amino acid sequence identity can be accomplished in a variety of known ways, for example, using publicly available computer software. Appropriate parameters for aligning the sequences can be determined, including any algorithms necessary to achieve maximum alignment over the full length of the sequences being compared.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The terms “effective amount” or “effective dose” as used herein have their plain and ordinary meaning as understood in light of the specification, and refer to that amount of a recited composition or compound that results in an observable effect. For example, an effective amount can refer to the amount of a composition or compound that improves a condition in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation typically suits the mode of administration.

Cryoprotectants are additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. For example, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, the term “treat” or “treating” or “treatment” shall have its plain and ordinary meaning and refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, curing the illness, etc. In some embodiments, treating can include one or more of preventing or protecting against the disease or disorder, causing the clinical symptoms not to develop, inhibiting the disease or disorder, arresting or suppressing the development of clinical symptoms, relieving the disease or disorder, and/or causing the regression of clinical symptoms. In certain embodiments, treatment of a subject achieves one, two, three, four, or more of the following effects, including, for example: (i) reduction or amelioration the severity of disease state or symptom associated therewith; (ii) reduction in the duration of a symptom associated with a disease or immune response; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalizations of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy.

As used herein, the term “tolerogen” refers to a molecule, composition, or substance that induces immune tolerance in an individual to that molecule, composition, or substance, or an antigenic portion thereof. Typically, when a substance (e.g., allergen, foreign peptide, organism, or an antigenic portion thereof) is introduced into an individual, an immune response is raised against the substance. This has implications in diseases, for example, auto-immune diseases (e.g., type 1 diabetes, multiple sclerosis), where the individual's immune system improperly reacts against a self-protein. Administration of a tolerogen (“tolerogenic therapy”) is intended to reduce or mitigate this unwanted immune response, either as a treatment or prophylaxis. The liver plays an important immunoregulatory role, as hepatocytes and other liver-resident cells exhibit the ability to uptake molecules, which may be normally antigenic, or abnormally antigenic in the case of auto-immune diseases, to induce immune tolerance.

As used herein, the term “liver-targeting moiety” refers to moieties having the ability to direct an agent (e.g., an immune tolerance inducing construct, a polypeptide, etc.) to the liver. The liver comprises different cell types, including but not limited to hepatocytes, sinusoidal epithelial cells, Kupffer cells, stellate cells, and/or dendritic cells. Typically, a liver-targeting moiety directs a polypeptide to one or more of these cells. On the surface of the respective liver cells, receptors are present which recognize and specifically bind the liver-targeting moiety. Liver-targeting can be achieved, for example, by chemical conjugation of an antigen or ligand to a galactosylating or glucosylating moiety, desialylation of an antigen or ligand to expose underlying galactosyl or glucosyl moieties, or specific binding of an antibody to an antigen or ligand associated with the liver, for example, ASGPR.

As used herein, the term “operably linked,” shall be given its ordinary meaning. In some embodiments, as an illustration, where two groups are operably linked, the groups are attached such that one or more of the linked groups is provided without substantial loss in its native reactivity or activity. In some embodiments, the antigens disclosed herein are operably linked to linking agents and targeting agents.

As used herein, the term “unwanted immune response” refers to a reaction by the immune system of a subject, which in the given situation is not desirable. Typically, a reaction of the immune system causes, enhances or worsens a disease if it is directed against an inappropriate target. For example, an unwanted immune response includes but is not limited to transplant rejection, immune response against a therapeutic agent, autoimmune disease, and allergy or hypersensitivity.

The term “variant” is to be understood as a protein (or nucleic acid) which differs in comparison to the protein (or nucleic acid chain) from which it is derived by one or more changes in its length, sequence, or structure. The polypeptide from which a protein variant is derived is also known as the parent polypeptide or polynucleotide. The term “variant” comprises “fragments” or “derivatives” of the parent molecule. Typically, “fragments” are smaller in length or size than the parent molecule, while “derivatives” exhibit one or more differences in their sequence or structure in comparison to the parent molecule. Also encompassed are modified molecules such as but not limited to post-translationally modified proteins (e.g. glycosylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant.” Naturally occurring and artificially constructed variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule (e.g., is functionally active). A variant can be characterized by a certain degree of sequence identity to the parent polypeptide from which it is derived. More precisely, a protein variant in the context of the present disclosure may exhibit at least 80% sequence identity to its parent polypeptide. Preferably, the sequence identity of protein variants is over a continuous stretch of 20, 30, 40, 45, 50, 60, 70, 80, 90, 100 or more amino acids. As discussed above, in several embodiments, variants exhibit about 80%, about 85%, about 90%, about 95%, about 97%, about 98%, about 99% functional equivalence to an unmodified or native reference sequence (and any degree of functional equivalence between those listed).

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

The disclosure herein generally uses affirmative language to describe the numerous embodiments. The disclosure also includes embodiments in which subject matter is excluded, in full or in part, such as substances or materials, method steps and conditions, protocols, or procedures.

### Liver-Specific Binding Polypeptides

Disclosed herein are compositions of binding polypeptides that bind to the asialoglycoprotein receptor (ASGPR) or a component thereof. In some embodiments, the component of the ASGPR is asialoglycoprotein receptor 1 (ASGR1) or asialoglycoprotein receptor 2 (ASGR2).

Additional anti-ASGPR antibodies are contemplated in U.S. Pat. No. 9,771,427 and 10,358,497, each of which is hereby expressly incorporated by reference in its entirety.

Additional liver-targeting compositions, which may or may not be polypeptides, for immune tolerance are explored in U.S. Pat. Nos. 10,046,056, 10,821,157, 10,940,209, 10,946,079, and 10,953,101, each of which is hereby expressly incorporated by reference in its entirety. Further liver-targeting compositions, which may or may not be polypeptides, for immune tolerance are explored in WO 2021/053589, which is hereby expressly incorporated by reference in its entirety.

Provided herein are ASGR1 binding polypeptides. In some embodiments, the ASGR1 binding polypeptides comprises a heavy chain variable region. In some embodiments, the heavy chain variable region comprises a heavy chain complementarity determining region 1 (HCDR1). In some embodiments, the heavy chain variable region comprises a HCDR2. In some embodiments, the heavy chain variable region comprises an HCDR3. In some embodiments, the heavy chain variable region comprises HCDR1, HCDR2, and HCDR3. In some embodiments, the HCDR1 comprises the sequence of SEQ ID NO: 9. In some embodiments, the HCDR1 comprises the sequence of SEQ ID NO: 297. In some embodiments, the HCDR1 comprises the sequence of SEQ ID NO: 329. In some embodiments, the HCDR1 comprises the sequence of SEQ ID NO: 361. In some embodiments, the HCDR1 comprises the sequence of SEQ ID NO: 393. In some embodiments, the HCDR1 comprises the sequence of SEQ ID NO: 425. In some embodiments, the HCDR1 comprises the sequence of SEQ ID NO: 457. In some embodiments, the HCDR1 comprises the sequence of SEQ ID NO: 489. In some embodiments, the HCDR1 comprises the sequence of SEQ ID NO: 521. In some embodiments, the HCDR1 comprises the sequence of SEQ ID NO: 553. In some embodiments, the HCDR2 comprises the sequence of SEQ ID NO: 10. In some embodiments, the HCDR2 comprises the sequence of SEQ ID NO: 298. In some embodiments, the HCDR2 comprises the sequence of SEQ ID NO: 330. In some embodiments, the HCDR2 comprises the sequence of SEQ ID NO: 362. In some embodiments, the HCDR2 comprises the sequence of SEQ ID NO: 394. In some embodiments, the HCDR2 comprises the sequence of SEQ ID NO: 426. In some embodiments, the HCDR2 comprises the sequence of SEQ ID NO: 458. In some embodiments, the HCDR2 comprises the sequence of SEQ ID NO: 490. In some embodiments, the HCDR2 comprises the sequence of SEQ ID NO: 522. In some embodiments, the HCDR2 comprises the sequence of SEQ ID NO: 554. In some embodiments, the HCDR3 comprises the sequence of SEQ ID NO: 11. In some embodiments, the HCDR3 comprises the sequence of SEQ ID NO: 299. In some embodiments, the HCDR3 comprises the sequence of SEQ ID NO: 331. In some embodiments, the HCDR3 comprises the sequence of SEQ ID NO: 363. In some embodiments, the HCDR3 comprises the sequence of SEQ ID NO: 395. In some embodiments, the HCDR3 comprises the sequence of SEQ ID NO: 427. In some embodiments, the HCDR3 comprises the sequence of SEQ ID NO: 459. In some embodiments, the HCDR3 comprises the sequence of SEQ ID NO: 491. In some embodiments, the HCDR3 comprises the sequence of SEQ ID NO: 523. In some embodiments, the HCDR3 comprises the sequence of SEQ ID NO: 555. In some embodiments, the HCDR1 comprises the sequence of SEQ ID NO: 9, 297, 329, 361, 393, 425, 457, 489, 521, or 553, the HCDR2 comprises the sequence of SEQ ID NO: 10, 298, 330, 362, 394, 426, 458, 490, 522, or 554 and the HCDR3 comprises the sequence of SEQ ID NO: 11, 299, 331, 363, 395, 427, 459, 491, 523, or 555. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 15. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 303. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 335. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 367. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 399. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 431. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 463. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 495. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 527. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 559. In some embodiments, the ASGR1 binding polypeptides comprise (or further comprise in combination with the heavy chain variable region) a light chain variable region. In some embodiments, the light chain variable region comprises a light chain CDR1 (LCDR1). In some embodiments, the light chain variable region comprises an LCDR2. In some embodiments, the light chain variable region comprises an LCDR3. In some embodiments, the light chain variable region comprises light chain CDRs LCDR1, LCDR2, and LCDR3. In some embodiments, the LCDR1 comprises the sequence of SEQ ID NO: 25. In some embodiments, the LCDR1 comprises the sequence of SEQ ID NO: 313. In some embodiments, the LCDR1 comprises the sequence of SEQ ID NO: 345. In some embodiments, the LCDR1 comprises the sequence of SEQ ID NO: 377. In some embodiments, the LCDR1 comprises the sequence of SEQ ID NO: 409. In some embodiments, the LCDR1 comprises the sequence of SEQ ID NO: 441. In some embodiments, the LCDR1 comprises the sequence of SEQ ID NO: 473. In some embodiments, the LCDR1 comprises the sequence of SEQ ID NO: 505. In some embodiments, the LCDR1 comprises the sequence of SEQ ID NO: 537. In some embodiments, the LCDR1 comprises the sequence of SEQ ID NO: 569. In some embodiments, the LCDR2 comprises the sequence of SEQ ID NO: 26. In some embodiments, the LCDR2 comprises the sequence of SEQ ID NO: 314. In some embodiments, the LCDR2 comprises the sequence of SEQ ID NO: 346. In some embodiments, the LCDR2 comprises the sequence of SEQ ID NO: 378. In some embodiments, the LCDR2 comprises the sequence of SEQ ID NO: 410. In some embodiments, the LCDR2 comprises the sequence of SEQ ID NO: 442. In some embodiments, the LCDR2 comprises the sequence of SEQ ID NO: 474. In some embodiments, the LCDR2 comprises the sequence of SEQ ID NO: 506. In some embodiments, the LCDR2 comprises the sequence of SEQ ID NO: 538. In some embodiments, the LCDR2 comprises the sequence of SEQ ID NO: 570. In some embodiments, the LCDR3 comprises the sequence of SEQ ID NO: 27. In some embodiments, the LCDR3 comprises the sequence of SEQ ID NO: 315. In some embodiments, the LCDR3 comprises the sequence of SEQ ID NO: 347. In some embodiments, the LCDR3 comprises the sequence of SEQ ID NO: 379. In some embodiments, the LCDR3 comprises the sequence of SEQ ID NO: 411. In some embodiments, the LCDR3 comprises the sequence of SEQ ID NO: 443. In some embodiments, the LCDR3 comprises the sequence of SEQ ID NO: 475. In some embodiments, the LCDR3 comprises the sequence of SEQ ID NO: 507. In some embodiments, the LCDR3 comprises the sequence of SEQ ID NO: 539. In some embodiments, the LCDR3 comprises the sequence of SEQ ID NO: 571. In some embodiments, the LCDR1 comprises the sequence of SEQ ID NO: 25, 313, 345, 377, 409, 441, 473, 505, 537, or 569, the LCDR2 comprises the sequence of SEQ ID NO: 26, 314, 346, 378, 410, 442, 474, 506, 538, or 570 and the LCDR3 comprises the sequence of SEQ ID NO: 27, 315, 347, 379, 411, 443, 475, 507, 539, or 571. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 31. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 319. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 351. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 383. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 415. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 447. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 479. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 511. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 543. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 575. In some embodiments, the heavy chain variable region comprises the sequence of SEQ ID NO: 15 and the light chain variable region comprises the sequence of SEQ ID NO: 31. In some embodiments, the heavy chain variable region comprises the sequence of SEQ ID NO: 303 and the light chain variable region comprises the sequence of SEQ ID NO: 319. In some embodiments, the heavy chain variable region comprises the sequence of SEQ ID NO: 335 and the light chain variable region comprises the sequence of SEQ ID NO: 351. In some embodiments, the heavy chain variable region comprises the sequence of SEQ ID NO: 367 and the light chain variable region comprises the sequence of SEQ ID NO: 383. In some embodiments, the heavy chain variable region comprises the sequence of SEQ ID NO: 399 and the light chain variable region comprises the sequence of SEQ ID NO: 415. In some embodiments, the heavy chain variable region comprises the sequence of SEQ ID NO: 431 and the light chain variable region comprises the sequence of SEQ ID NO: 447. In some embodiments, the heavy chain variable region comprises the sequence of SEQ ID NO: 463 and the light chain variable region comprises the sequence of SEQ ID NO: 479. In some embodiments, the heavy chain variable region comprises the sequence of SEQ ID NO: 495 and the light chain variable region comprises the sequence of SEQ ID NO: 511. In some embodiments, the heavy chain variable region comprises the sequence of SEQ ID NO: 527 and the light chain variable region comprises the sequence of SEQ ID NO: 543. In some embodiments, the heavy chain variable region comprises the sequence of SEQ ID NO: 559 and the light chain variable region comprises the sequence of SEQ ID NO: 575. In some embodiments, the ASGR1 binding polypeptide is an antibody, an Fab′ fragment, an F(ab′)2 fragment, a domain antibody (dAb), or an scFv. In some embodiments, the ASGR1 binding polypeptide comprises an Fc domain. In some embodiments, the Fc domain is silenced. In some embodiments, the Fc domain is partially silenced.

Also disclosed herein are polynucleotides encoding for an ASGR1 binding polypeptide. In some embodiments, the polynucleotide comprises one or more of the sequences of SEQ ID NO: 12-14 or 25-27. In some embodiments, the polynucleotide comprises one or more of the sequences of SEQ ID NO: 300-302 or 313-315. In some embodiments, the polynucleotide comprises one or more of the sequences of SEQ ID NO: 332-334 or 345-347. In some embodiments, the polynucleotide comprises one or more of the sequences of SEQ ID NO: 364-366 or 377-379. In some embodiments, the polynucleotide comprises one or more of the sequences of SEQ ID NO: 396-398 or 409-411. In some embodiments, the polynucleotide comprises one or more of the sequences of SEQ ID NO: 428-430 or 441-443. In some embodiments, the polynucleotide comprises one or more of the sequences of SEQ ID NO: 460-462 or 473-475. In some embodiments, the polynucleotide comprises one or more of the sequences of SEQ ID NO: 492-494 or 505-507. In some embodiments, the polynucleotide comprises one or more of the sequences of SEQ ID NO: 524-526 or 537-539. In some embodiments, the polynucleotide comprises one or more of the sequences of SEQ ID NO: 556-558 or 569-571. In some embodiments, the polynucleotide comprises the sequence of SEQ ID NO: 16, which is a non-limiting example of a polynucleotide sequence that encodes for the heavy chain variable region sequence of SEQ ID NO: 15. In some embodiments, the polynucleotide comprises the sequence of SEQ ID NO: 304, which is a non-limiting example of a polynucleotide sequence that encodes for the heavy chain variable region sequence of SEQ ID NO: 303. In some embodiments, the polynucleotide comprises the sequence of SEQ ID NO: 336, which is a non-limiting example of a polynucleotide sequence that encodes for the heavy chain variable region sequence of SEQ ID NO: 335. In some embodiments, the polynucleotide comprises the sequence of SEQ ID NO: 368, which is a non-limiting example of a polynucleotide sequence that encodes for the heavy chain variable region sequence of SEQ ID NO: 367. In some embodiments, the polynucleotide comprises the sequence of SEQ ID NO: 400, which is a non-limiting example of a polynucleotide sequence that encodes for the heavy chain variable region sequence of SEQ ID NO: 399. In some embodiments, the polynucleotide comprises the sequence of SEQ ID NO: 432, which is a non-limiting example of a polynucleotide sequence that encodes for the heavy chain variable region sequence of SEQ ID NO: 431. In some embodiments, the polynucleotide comprises the sequence of SEQ ID NO: 464, which is a non-limiting example of a polynucleotide sequence that encodes for the heavy chain variable region sequence of SEQ ID NO: 463. In some embodiments, the polynucleotide comprises the sequence of SEQ ID NO: 496, which is a non-limiting example of a polynucleotide sequence that encodes for the heavy chain variable region sequence of SEQ ID NO: 495. In some embodiments, the polynucleotide comprises the sequence of SEQ ID NO: 528, which is a non-limiting example of a polynucleotide sequence that encodes for the heavy chain variable region sequence of SEQ ID NO: 527. In some embodiments, the polynucleotide comprises the sequence of SEQ ID NO: 560, which is a non-limiting example of a polynucleotide sequence that encodes for the heavy chain variable region sequence of SEQ ID NO: 559. In some embodiments, the polynucleotide comprises the sequence of SEQ ID NO: 32, which is a non-limiting example of a polynucleotide sequence that encodes for the light chain variable region sequence of SEQ ID NO: 31. In some embodiments, the polynucleotide comprises the sequence of SEQ ID NO: 320, which is a non-limiting example of a polynucleotide sequence that encodes for the light chain variable region sequence of SEQ ID NO: 319. In some embodiments, the polynucleotide comprises the sequence of SEQ ID NO: 352, which is a non-limiting example of a polynucleotide sequence that encodes for the light chain variable region sequence of SEQ ID NO: 351. In some embodiments, the polynucleotide comprises the sequence of SEQ ID NO: 384, which is a non-limiting example of a polynucleotide sequence that encodes for the light chain variable region sequence of SEQ ID NO: 383. In some embodiments, the polynucleotide comprises the sequence of SEQ ID NO: 416, which is a non-limiting example of a polynucleotide sequence that encodes for the light chain variable region sequence of SEQ ID NO: 415. In some embodiments, the polynucleotide comprises the sequence of SEQ ID NO: 448, which is a non-limiting example of a polynucleotide sequence that encodes for the light chain variable region sequence of SEQ ID NO: 447. In some embodiments, the polynucleotide comprises the sequence of SEQ ID NO: 480, which is a non-limiting example of a polynucleotide sequence that encodes for the light chain variable region sequence of SEQ ID NO: 479. In some embodiments, the polynucleotide comprises the sequence of SEQ ID NO: 512, which is a non-limiting example of a polynucleotide sequence that encodes for the light chain variable region sequence of SEQ ID NO: 511. In some embodiments, the polynucleotide comprises the sequence of SEQ ID NO: 544, which is a non-limiting example of a polynucleotide sequence that encodes for the light chain variable region sequence of SEQ ID NO: 543. In some embodiments, the polynucleotide comprises the sequence of SEQ ID NO: 576, which is a non-limiting example of a polynucleotide sequence that encodes for the light chain variable region sequence of SEQ ID NO: 575. In some embodiments, the polynucleotide encodes for any one of the ASGR1 binding polynucleotides disclosed herein.

As applied to the ASGR1 binding polypeptides and polynucleotides that encode therefor disclosed herein, the ASGR1 binding polypeptides may further comprise immunoglobulin frameworks. These immunoglobulin frameworks may be conventionally known in the art. For example, in several embodiments, each heavy chain variable region and light chain variable region framework used has 4 framework sequences (FW-1, FW-2, FW-3, FW-4) in which the three CDRs are provided.

A non-limiting example of a heavy chain framework 1 (H-FR1) is provided in SEQ ID NO: 1 and may be encoded by the polynucleotide provided in SEQ ID NO: 5. Another non-limiting example of a heavy chain framework 1 (H-FR1) is provided in SEQ ID NO: 289 and may be encoded by the polynucleotide provided in SEQ ID NO: 293. Another non-limiting example of a heavy chain framework 1 (H-FR1) is provided in SEQ ID NO: 321 and may be encoded by the polynucleotide provided in SEQ ID NO: 325. Another non-limiting example of a heavy chain framework 1 (H-FR1) is provided in SEQ ID NO: 353 and may be encoded by the polynucleotide provided in SEQ ID NO: 357. Another non-limiting example of a heavy chain framework 1 (H-FR1) is provided in SEQ ID NO: 385 and may be encoded by the polynucleotide provided in SEQ ID NO: 389. Another non-limiting example of a heavy chain framework 1 (H-FR1) is provided in SEQ ID NO: 417 and may be encoded by the polynucleotide provided in SEQ ID NO: 421. Another non-limiting example of a heavy chain framework 1 (H-FR1) is provided in SEQ ID NO: 449 and may be encoded by the polynucleotide provided in SEQ ID NO: 453. Another non-limiting example of a heavy chain framework 1 (H-FR1) is provided in SEQ ID NO: 481 and may be encoded by the polynucleotide provided in SEQ ID NO: 485. Another non-limiting example of a heavy chain framework 1 (H-FR1) is provided in SEQ ID NO: 513 and may be encoded by the polynucleotide provided in SEQ ID NO: 517. Another non-limiting example of a heavy chain framework 1 (H-FR1) is provided in SEQ ID NO: 545 and may be encoded by the polynucleotide provided in SEQ ID NO: 549. A non-limiting example of a heavy chain framework 2 (H-FR2) is provided in SEQ ID NO: 2 and may be encoded by the polynucleotide provided in SEQ ID NO: 6. A non-limiting example of a heavy chain framework 2 (H-FR2) is provided in SEQ ID NO: 290 and may be encoded by the polynucleotide provided in SEQ ID NO: 294. A non-limiting example of a heavy chain framework 2 (H-FR2) is provided in SEQ ID NO: 322 and may be encoded by the polynucleotide provided in SEQ ID NO: 326. A non-limiting example of a heavy chain framework 2 (H-FR2) is provided in SEQ ID NO: 354 and may be encoded by the polynucleotide provided in SEQ ID NO: 358. A non-limiting example of a heavy chain framework 2 (H-FR2) is provided in SEQ ID NO: 386 and may be encoded by the polynucleotide provided in SEQ ID NO: 390. A non-limiting example of a heavy chain framework 2 (H-FR2) is provided in SEQ ID NO: 418 and may be encoded by the polynucleotide provided in SEQ ID NO: 422. A non-limiting example of a heavy chain framework 2 (H-FR2) is provided in SEQ ID NO: 450 and may be encoded by the polynucleotide provided in SEQ ID NO: 454. A non-limiting example of a heavy chain framework 2 (H-FR2) is provided in SEQ ID NO: 482 and may be encoded by the polynucleotide provided in SEQ ID NO: 486. A non-limiting example of a heavy chain framework 2 (H-FR2) is provided in SEQ ID NO: 514 and may be encoded by the polynucleotide provided in SEQ ID NO: 518. A non-limiting example of a heavy chain framework 2 (H-FR2) is provided in SEQ ID NO: 546 and may be encoded by the polynucleotide provided in SEQ ID NO: 550. A non-limiting example of a heavy chain framework 3 (H-FR3) is provided in SEQ ID NO: 3 and may be encoded by the polynucleotide provided in SEQ ID NO: 7. A non-limiting example of a heavy chain framework 3 (H-FR3) is provided in SEQ ID NO: 291 and may be encoded by the polynucleotide provided in SEQ ID NO: 295. A non-limiting example of a heavy chain framework 3 (H-FR3) is provided in SEQ ID NO: 323 and may be encoded by the polynucleotide provided in SEQ ID NO: 327. A non-limiting example of a heavy chain framework 3 (H-FR3) is provided in SEQ ID NO: 355 and may be encoded by the polynucleotide provided in SEQ ID NO: 359. A non-limiting example of a heavy chain framework 3 (H-FR3) is provided in SEQ ID NO: 387 and may be encoded by the polynucleotide provided in SEQ ID NO: 391. A non-limiting example of a heavy chain framework 3 (H-FR3) is provided in SEQ ID NO: 419 and may be encoded by the polynucleotide provided in SEQ ID NO: 423. A non-limiting example of a heavy chain framework 3 (H-FR3) is provided in SEQ ID NO: 451 and may be encoded by the polynucleotide provided in SEQ ID NO: 455. A non-limiting example of a heavy chain framework 3 (H-FR3) is provided in SEQ ID NO: 483 and may be encoded by the polynucleotide provided in SEQ ID NO: 487. A non-limiting example of a heavy chain framework 3 (H-FR3) is provided in SEQ ID NO: 515 and may be encoded by the polynucleotide provided in SEQ ID NO: 519. A non-limiting example of a heavy chain framework 3 (H-FR3) is provided in SEQ ID NO: 547 and may be encoded by the polynucleotide provided in SEQ ID NO: 551. A non-limiting example of a heavy chain framework 4 (H-FR4) is provided in SEQ ID NO: 4 and may be encoded by the polynucleotide provided in SEQ ID NO: 8. A non-limiting example of a heavy chain framework 4 (H-FR4) is provided in SEQ ID NO: 292 and may be encoded by the polynucleotide provided in SEQ ID NO: 296. A non-limiting example of a heavy chain framework 4 (H-FR4) is provided in SEQ ID NO: 324 and may be encoded by the polynucleotide provided in SEQ ID NO: 328. A non-limiting example of a heavy chain framework 4 (H-FR4) is provided in SEQ ID NO: 356 and may be encoded by the polynucleotide provided in SEQ ID NO: 360. A non-limiting example of a heavy chain framework 4 (H-FR4) is provided in SEQ ID NO: 388 and may be encoded by the polynucleotide provided in SEQ ID NO: 392. A non-limiting example of a heavy chain framework 4 (H-FR4) is provided in SEQ ID NO: 420 and may be encoded by the polynucleotide provided in SEQ ID NO: 424. A non-limiting example of a heavy chain framework 4 (H-FR4) is provided in SEQ ID NO: 452 and may be encoded by the polynucleotide provided in SEQ ID NO: 456. A non-limiting example of a heavy chain framework 4 (H-FR4) is provided in SEQ ID NO: 484 and may be encoded by the polynucleotide provided in SEQ ID NO: 488. A non-limiting example of a heavy chain framework 4 (H-FR4) is provided in SEQ ID NO: 516 and may be encoded by the polynucleotide provided in SEQ ID NO: 520. A non-limiting example of a heavy chain framework 4 (H-FR4) is provided in SEQ ID NO: 548 and may be encoded by the polynucleotide provided in SEQ ID NO: 552. A non-limiting example of a light chain framework 1 (L-FR1) is provided in SEQ ID NO: 17 and may be encoded by the polynucleotide provided in SEQ ID NO: 21. A non-limiting example of a light chain framework 1 (L-FR1) is provided in SEQ ID NO: 305 and may be encoded by the polynucleotide provided in SEQ ID NO: 309. A non-limiting example of a light chain framework 1 (L-FR1) is provided in SEQ ID NO: 337 and may be encoded by the polynucleotide provided in SEQ ID NO: 341. A non-limiting example of a light chain framework 1 (L-FR1) is provided in SEQ ID NO: 369 and may be encoded by the polynucleotide provided in SEQ ID NO: 373. A non-limiting example of a light chain framework 1 (L-FR1) is provided in SEQ ID NO: 401 and may be encoded by the polynucleotide provided in SEQ ID NO: 405. A non-limiting example of a light chain framework 1 (L-FR1) is provided in SEQ ID NO: 433 and may be encoded by the polynucleotide provided in SEQ ID NO: 437. A non-limiting example of a light chain framework 1 (L-FR1) is provided in SEQ ID NO: 465 and may be encoded by the polynucleotide provided in SEQ ID NO: 469. A non-limiting example of a light chain framework 1 (L-FR1) is provided in SEQ ID NO: 497 and may be encoded by the polynucleotide provided in SEQ ID NO: 501. A non-limiting example of a light chain framework 1 (L-FR1) is provided in SEQ ID NO: 529 and may be encoded by the polynucleotide provided in SEQ ID NO: 533. A non-limiting example of a light chain framework 1 (L-FR1) is provided in SEQ ID NO: 561 and may be encoded by the polynucleotide provided in SEQ ID NO: 565. A non-limiting example of a light chain framework 2 (L-FR2) is provided in SEQ ID NO: 18 and may be encoded by the polynucleotide provided in SEQ ID NO: 22. A non-limiting example of a light chain framework 2 (L-FR2) is provided in SEQ ID NO: 306 and may be encoded by the polynucleotide provided in SEQ ID NO: 310. A non-limiting example of a light chain framework 2 (L-FR2) is provided in SEQ ID NO: 338 and may be encoded by the polynucleotide provided in SEQ ID NO: 342. A non-limiting example of a light chain framework 2 (L-FR2) is provided in SEQ ID NO: 370 and may be encoded by the polynucleotide provided in SEQ ID NO: 374. A non-limiting example of a light chain framework 2 (L-FR2) is provided in SEQ ID NO: 402 and may be encoded by the polynucleotide provided in SEQ ID NO: 406. A non-limiting example of a light chain framework 2 (L-FR2) is provided in SEQ ID NO: 434 and may be encoded by the polynucleotide provided in SEQ ID NO: 438. A non-limiting example of a light chain framework 2 (L-FR2) is provided in SEQ ID NO: 466 and may be encoded by the polynucleotide provided in SEQ ID NO: 470. A non-limiting example of a light chain framework 2 (L-FR2) is provided in SEQ ID NO: 498 and may be encoded by the polynucleotide provided in SEQ ID NO: 502. A non-limiting example of a light chain framework 2 (L-FR2) is provided in SEQ ID NO: 530 and may be encoded by the polynucleotide provided in SEQ ID NO: 534. A non-limiting example of a light chain framework 2 (L-FR2) is provided in SEQ ID NO: 562 and may be encoded by the polynucleotide provided in SEQ ID NO: 566. A non-limiting example of a light chain framework 3 (L-FR3) is provided in SEQ ID NO: 19 and may be encoded by the polynucleotide provided in SEQ ID NO: 23. A non-limiting example of a light chain framework 3 (L-FR3) is provided in SEQ ID NO: 307 and may be encoded by the polynucleotide provided in SEQ ID NO: 311. A non-limiting example of a light chain framework 3 (L-FR3) is provided in SEQ ID NO: 339 and may be encoded by the polynucleotide provided in SEQ ID NO: 343. A non-limiting example of a light chain framework 3 (L-FR3) is provided in SEQ ID NO: 371 and may be encoded by the polynucleotide provided in SEQ ID NO: 375. A non-limiting example of a light chain framework 3 (L-FR3) is provided in SEQ ID NO: 403 and may be encoded by the polynucleotide provided in SEQ ID NO: 407. A non-limiting example of a light chain framework 3 (L-FR3) is provided in SEQ ID NO: 435 and may be encoded by the polynucleotide provided in SEQ ID NO: 439. A non-limiting example of a light chain framework 3 (L-FR3) is provided in SEQ ID NO: 467 and may be encoded by the polynucleotide provided in SEQ ID NO: 471. A non-limiting example of a light chain framework 3 (L-FR3) is provided in SEQ ID NO: 499 and may be encoded by the polynucleotide provided in SEQ ID NO: 503. A non-limiting example of a light chain framework 3 (L-FR3) is provided in SEQ ID NO: 531 and may be encoded by the polynucleotide provided in SEQ ID NO: 535. A non-limiting example of a light chain framework 3 (L-FR3) is provided in SEQ ID NO: 563 and may be encoded by the polynucleotide provided in SEQ ID NO: 567. A non-limiting example of a light chain framework 4 (L-FR4) is provided in SEQ ID NO: 20 and may be encoded by the polynucleotide provided in SEQ ID NO: 24. A non-limiting example of a light chain framework 4 (L-FR4) is provided in SEQ ID NO: 308 and may be encoded by the polynucleotide provided in SEQ ID NO: 312. A non-limiting example of a light chain framework 4 (L-FR4) is provided in SEQ ID NO: 340 and may be encoded by the polynucleotide provided in SEQ ID NO: 344. A non-limiting example of a light chain framework 4 (L-FR4) is provided in SEQ ID NO: 372 and may be encoded by the polynucleotide provided in SEQ ID NO: 376. A non-limiting example of a light chain framework 4 (L-FR4) is provided in SEQ ID NO: 404 and may be encoded by the polynucleotide provided in SEQ ID NO: 408. A non-limiting example of a light chain framework 4 (L-FR4) is provided in SEQ ID NO: 436 and may be encoded by the polynucleotide provided in SEQ ID NO: 440. A non-limiting example of a light chain framework 4 (L-FR4) is provided in SEQ ID NO: 468 and may be encoded by the polynucleotide provided in SEQ ID NO: 472. A non-limiting example of a light chain framework 4 (L-FR4) is provided in SEQ ID NO: 500 and may be encoded by the polynucleotide provided in SEQ ID NO: 504. A non-limiting example of a light chain framework 4 (L-FR4) is provided in SEQ ID NO: 532 and may be encoded by the polynucleotide provided in SEQ ID NO: 536. A non-limiting example of a light chain framework 4 (L-FR4) is provided in SEQ ID NO: 564 and may be encoded by the polynucleotide provided in SEQ ID NO: 568. It is envisioned that alternative frameworks may be substituted for any of the frameworks disclosed herein as understood by one skilled in the art.

In some embodiments, the ASGR1 binding polypeptides disclosed herein bind to ASGR1. By virtue of binding to ASGR1, these ASGR1 binding polypeptides can also bind to the asialoglycoprotein receptor complex (ASGPR), which is composed of ASGR1 and ASGR2 subunits. In some embodiments, the ASGR1 binding polypeptides provided herein do not bind to ASGR2 (e.g., they are specific to ASGR1).

### Tolerogenic Compounds and Compositions

Embodiments disclosed herein relate to compounds, compositions (e.g., pharmaceutical compositions) or constructs for immune tolerance. Immune tolerance can be induced against a variety of antigens, based on the disclosed provided herein. For example, the antigen can be endogenous (e.g., an autoantigen) or exogenous (e.g., a foreign antigen or an alloantigen), including but not limited to: an alloantigen against which transplant recipients develop an unwanted immune response, such as graft-vs-host disease or transplant rejection, a foreign food, animal, plant, or environmental antigen to which patients develop an unwanted immune response, such as allergy or hypersensitivity, a therapeutic agent to which patients develop an unwanted immune response, such as hypersensitivity and/or reduced therapeutic activity, an autoantigen to which patients develop an unwanted immune response, such as an autoimmune disease, or a tolerogenic portion, such as a fragment or epitope, of any such type of antigen.

Disclosed herein are tolerogenic compounds. In some embodiments, the tolerogenic compounds comprise an ASGR1 binding polypeptide conjugated or fused to an antigen to which tolerance is desired. In some embodiments, the ASGR1 binding polypeptide and antigen are conjugated or fused with a linker, optionally a peptide linker or chemical conjugation linker. In some embodiments, the ASGR1 binding polypeptide is chemically conjugated to the antigen. In some embodiments, the ASGR1 binding polypeptide is recombinantly fused to the antigen. In some embodiments, the ASGR1 binding polypeptide is any one of the ASGR1 binding polypeptides disclosed herein. In some embodiments, the ASGR1 binding polypeptide comprises a heavy chain variable region. In some embodiments, the heavy chain variable region comprises one or more of heavy chain CDRs HCDR1, HCDR2, and HCDR3. In some embodiments, the HCDR1 comprises the sequence of SEQ ID NO: 9. In some embodiments, the HCDR1 comprises the sequence of SEQ ID NO: 297. In some embodiments, the HCDR1 comprises the sequence of SEQ ID NO: 329. In some embodiments, the HCDR1 comprises the sequence of SEQ ID NO: 361. In some embodiments, the HCDR1 comprises the sequence of SEQ ID NO: 393. In some embodiments, the HCDR1 comprises the sequence of SEQ ID NO: 425. In some embodiments, the HCDR1 comprises the sequence of SEQ ID NO: 457. In some embodiments, the HCDR1 comprises the sequence of SEQ ID NO: 489. In some embodiments, the HCDR1 comprises the sequence of SEQ ID NO: 521. In some embodiments, the HCDR1 comprises the sequence of SEQ ID NO: 553. In some embodiments, the HCDR2 comprises the sequence of SEQ ID NO: 10. In some embodiments, the HCDR2 comprises the sequence of SEQ ID NO: 298. In some embodiments, the HCDR2 comprises the sequence of SEQ ID NO: 330. In some embodiments, the HCDR2 comprises the sequence of SEQ ID NO: 362. In some embodiments, the HCDR2 comprises the sequence of SEQ ID NO: 394. In some embodiments, the HCDR2 comprises the sequence of SEQ ID NO: 426. In some embodiments, the HCDR2 comprises the sequence of SEQ ID NO: 458. In some embodiments, the HCDR2 comprises the sequence of SEQ ID NO: 490. In some embodiments, the HCDR2 comprises the sequence of SEQ ID NO: 522. In some embodiments, the HCDR2 comprises the sequence of SEQ ID NO: 554. In some embodiments, the HCDR3 comprises the sequence of SEQ ID NO: 11. In some embodiments, the HCDR3 comprises the sequence of SEQ ID NO: 299. In some embodiments, the HCDR3 comprises the sequence of SEQ ID NO: 331. In some embodiments, the HCDR3 comprises the sequence of SEQ ID NO: 363. In some embodiments, the HCDR3 comprises the sequence of SEQ ID NO: 395. In some embodiments, the HCDR3 comprises the sequence of SEQ ID NO: 427. In some embodiments, the HCDR3 comprises the sequence of SEQ ID NO: 459. In some embodiments, the HCDR3 comprises the sequence of SEQ ID NO: 491. In some embodiments, the HCDR3 comprises the sequence of SEQ ID NO: 523. In some embodiments, the HCDR3 comprises the sequence of SEQ ID NO: 555. In some embodiments, the HCDR1 comprises the sequence of SEQ ID NO: 9, 297, 329, 361, 393, 425, 457, 489, 521, or 553, the HCDR2 comprises the sequence of SEQ ID NO: 10, 298, 330, 362, 394, 426, 458, 490, 522, or 554 and the HCDR3 comprises the sequence of SEQ ID NO: 11, 299, 331, 363, 395, 427, 459, 491, 523, or 555. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 15. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 303. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 335. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 367. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 399. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 431. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 463. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 495. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 527. In some embodiments, the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 559. In some embodiments, the ASGR 1 binding polypeptide comprises (or further comprises in combination with the heavy chain variable region) a light chain variable region. In some embodiments, the light chain variable region comprises one or more of light chain CDRs LCDR1, LCDR2, and LCDR3. In some embodiments, the LCDR1 comprises the sequence of SEQ ID NO: 25. In some embodiments, the LCDR1 comprises the sequence of SEQ ID NO: 313. In some embodiments, the LCDR1 comprises the sequence of SEQ ID NO: 345. In some embodiments, the LCDR1 comprises the sequence of SEQ ID NO: 377. In some embodiments, the LCDR1 comprises the sequence of SEQ ID NO: 409. In some embodiments, the LCDR1 comprises the sequence of SEQ ID NO: 441. In some embodiments, the LCDR1 comprises the sequence of SEQ ID NO: 473. In some embodiments, the LCDR1 comprises the sequence of SEQ ID NO: 505. In some embodiments, the LCDR1 comprises the sequence of SEQ ID NO: 537. In some embodiments, the LCDR1 comprises the sequence of SEQ ID NO: 569. In some embodiments, the LCDR2 comprises the sequence of SEQ ID NO: 26 In some embodiments, the LCDR2 comprises the sequence of SEQ ID NO: 314. In some embodiments, the LCDR2 comprises the sequence of SEQ ID NO: 346. In some embodiments, the LCDR2 comprises the sequence of SEQ ID NO: 378. In some embodiments, the LCDR2 comprises the sequence of SEQ ID NO: 410. In some embodiments, the LCDR2 comprises the sequence of SEQ ID NO: 442. In some embodiments, the LCDR2 comprises the sequence of SEQ ID NO: 474. In some embodiments, the LCDR2 comprises the sequence of SEQ ID NO: 506. In some embodiments, the LCDR2 comprises the sequence of SEQ ID NO: 538. In some embodiments, the LCDR2 comprises the sequence of SEQ ID NO: 570. In some embodiments, the LCDR3 comprises the sequence of SEQ ID NO: 27. In some embodiments, the LCDR3 comprises the sequence of SEQ ID NO: 315. In some embodiments, the LCDR3 comprises the sequence of SEQ ID NO: 347. In some embodiments, the LCDR3 comprises the sequence of SEQ ID NO: 379. In some embodiments, the LCDR3 comprises the sequence of SEQ ID NO: 411. In some embodiments, the LCDR3 comprises the sequence of SEQ ID NO: 443. In some embodiments, the LCDR3 comprises the sequence of SEQ ID NO: 475. In some embodiments, the LCDR3 comprises the sequence of SEQ ID NO: 507. In some embodiments, the LCDR3 comprises the sequence of SEQ ID NO: 539. In some embodiments, the LCDR3 comprises the sequence of SEQ ID NO: 571. In some embodiments, the LCDR1 comprises the sequence of SEQ ID NO: 25, 313, 345, 377, 409, 441, 473, 505, 537, or 569, the LCDR2 comprises the sequence of SEQ ID NO: 26, 314, 346, 378, 410, 442, 474, 506, 538, or 570 and the LCDR3 comprises the sequence of SEQ ID NO: 27, 315, 347, 379, 411, 443, 475, 507, 539, or 571. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 31. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 319. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 351. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 383. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 415. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 447. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 479. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 511. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 543. In some embodiments, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 575. In some embodiments, the heavy chain variable region comprises the sequence of SEQ ID NO: 15 and the light chain variable region comprises the sequence of SEQ ID NO: 31. In some embodiments, the heavy chain variable region comprises the sequence of SEQ ID NO: 303 and the light chain variable region comprises the sequence of SEQ ID NO: 319. In some embodiments, the heavy chain variable region comprises the sequence of SEQ ID NO: 335 and the light chain variable region comprises the sequence of SEQ ID NO: 351. In some embodiments, the heavy chain variable region comprises the sequence of SEQ ID NO: 367 and the light chain variable region comprises the sequence of SEQ ID NO: 383. In some embodiments, the heavy chain variable region comprises the sequence of SEQ ID NO: 399 and the light chain variable region comprises the sequence of SEQ ID NO: 415. In some embodiments, the heavy chain variable region comprises the sequence of SEQ ID NO: 431 and the light chain variable region comprises the sequence of SEQ ID NO: 447. In some embodiments, the heavy chain variable region comprises the sequence of SEQ ID NO: 463 and the light chain variable region comprises the sequence of SEQ ID NO: 479. In some embodiments, the heavy chain variable region comprises the sequence of SEQ ID NO: 495 and the light chain variable region comprises the sequence of SEQ ID NO: 511. In some embodiments, the heavy chain variable region comprises the sequence of SEQ ID NO: 527 and the light chain variable region comprises the sequence of SEQ ID NO: 543. In some embodiments, the heavy chain variable region comprises the sequence of SEQ ID NO: 559 and the light chain variable region comprises the sequence of SEQ ID NO: 575. In some embodiments, the ASGR1 binding polypeptide is an antibody, an Fab′ fragment, an F(ab′)2 fragment, a domain antibody (dAb), or an scFv. In some embodiments, the ASGR1 binding polypeptide comprises an Fc domain. In some embodiments, the Fc domain is silenced. In some embodiments, the Fc domain is partially silenced.

In some embodiments of any of the tolerogenic compounds disclosed herein, the ASGR1 binding polypeptide and the antigen are conjugated or fused. In some embodiments, the ASGR1 binding polypeptide and the antigen are conjugated or fused with a linker. In some embodiments, the ASGR1 binding polypeptide and the antigen are chemically conjugated, such as with a chemical conjugation linker. In some embodiments, the ASGR1 binding polypeptide and the antigen are recombinantly fused. In some embodiments, the ASGR1 binding polypeptide and the antigen are recombinantly fused with a linker. In some embodiments, the linker is a polypeptide linker (e.g., including but not limited to when the ASGR1 binding polypeptide and the antigen are both polypeptides). In some embodiments, the linker is a cleavable linker. In some embodiments, the linker comprises glycine and/or serine. The use of glycine and serine linkers are generally known in the art. In some embodiments, the linker comprises the sequence of SEQ ID NO: 37. In some embodiments, the antigen is conjugated or fused to the N-terminus or C-terminus of the ASGR1 binding polypeptide, for example, the N-terminus or C-terminus of either the heavy chain, heavy chain variable region, light chain, or light chain variable region of the ASGR1 binding polypeptide when the ASGR1 binding polypeptide is a form of antibody. In some embodiments, the ASGR1 binding polypeptide comprises an Fc domain. In some embodiments, the Fc domain is silenced or partially silenced, for example, such that the Fc domain exhibits substantially reduced binding or no binding to FcRn or other FcRγ receptors. In some embodiments, the silencing of the Fc domain abrogates the recycling of the antibody back out of the cell after uptake. In some embodiments, the antigen is conjugated or fused to the Fc domain. In some embodiments, the antigen is conjugated or fused to the C-terminus of the Fc domain.

In some embodiments, the antigen of any one of the tolerogenic compounds disclosed herein is an agent that is capable of inducing an unwanted immune response in a subject. In some embodiments, the antigen can be a protein, peptide, or polypeptide. In some embodiments, the antigen can be a complete or partial therapeutic agent, a full-length autoantigen or portion thereof, a full-length alloantigen or portion thereof, a full-length allergen or portion thereof, or a mimetic of any of the aforementioned antigens. Also envisioned are nucleic acids, which may encode for any of the aforementioned antigens, or act as antigens themselves (for example, as nucleic acid autoantigens). Combinations of multiple antigens or portions or fragments may also be used, depending on the embodiment. For example, if a longer peptide identified as P has antigenic regions A, B, C, and D, compositions disclosed herein for induction of tolerance to P can comprise any one or more of A, B, C, and D, and/or repeats of any one or more of A, B, C, and D. A listing of any particular antigen in a category or association with any particular disease or reaction does not preclude that antigen from being considered part of another category or associated with another disease or reaction.

In several embodiments, the antigen to which tolerance is desired comprises one or more foreign antigens, such as food, animal, plant, and environmental antigens against which a patient experiences an unwanted immune response. While a therapeutic protein can also be considered a foreign antigen due to its exogenous origin, for purposes of clarity in the description of the present disclosure, such therapeutics are described as a separate group. Similarly, a plant or an animal antigen can be eaten and considered a food antigen, and an environmental antigen may originate from a plant. They are, however, considered foreign antigens. In the interest of simplicity, no attempt will be made to describe distinguish and define all of such potentially overlapping groups, as those skilled in the art can appreciate the antigens that can be employed in the compositions of the disclosure, particularly in light of the detailed description and examples.

In several embodiments, the antigen comprises one or more therapeutic agents that are proteins, peptides, antibodies, and antibody-like molecules (including antibody fragments and fusion proteins with antibodies and antibody fragments), and gene therapy vectors. These include human, non-human (such as mouse) and non-natural (e.g., engineered) proteins, antibodies, chimeric antibodies, humanized antibodies, viruses and virus-like particles, and non-antibody binding scaffolds, such as fibronectins, DARPins, knottins, and the like. In several embodiments, human allograft transplantation antigens against which transplant recipients develop an unwanted immune response are used. In several embodiments, the antigen comprises one or more autoantigens that cause an unwanted, autoimmune response. While autoantigens are of an endogenous origin in an autoimmune disease patient, according to several embodiments, the polypeptides employed in the disclosed compositions are, depending on the embodiment, synthesized exogenously (as opposed to being purified and concentrated from a source of origin).

In several embodiments, the antigen to which tolerance is desired comprises one or more foreign antigens, such as food, animal, plant, and environmental antigens against which a patient experiences an unwanted immune response. While a therapeutic protein can also be considered a foreign antigen due to its exogenous origin, for purposes of clarity in the description of the present disclosure such therapeutics are described as a separate group. Similarly, a plant or an animal antigen can be eaten and considered a food antigen, and an environmental antigen may originate from a plant. They are, however, considered foreign antigens. In the interest of simplicity no attempt will be made to describe distinguish and define all of such potentially overlapping groups, as those skilled in the art can appreciate the antigens that can be employed in the compositions of the disclosure, particularly in light of the detailed description and examples.

In several embodiments, the antigen is selected from the group consisting of insulin, proinsulin, preproinsulin, gluten, gliadin, myelin basic protein, myelin oligodendrocyte glycoprotein and proteolipid protein, desmoglein-3, desmoglein-1, alpha-synuclein, acetylcholine receptor, Factor VIII, Factor IX, asparaginase, uricase, adeno-associated viruses (AAV), and fragments of any of the preceding. In several embodiments, the antigen is not a full-length protein. For example, in some embodiments, the antigen is not full-length gliadin, insulin, or proinsulin. In several embodiments, the antigen is not full-length myelin basic protein, not full-length myelin oligodendrocyte protein, or not full-length proteolipid protein. In several embodiments, the antigen is not a fragment of a protein. As discussed in more detail below, there exist a variety of antigens to which tolerance may be desired. These may include, but are not limited to, exogenous antigens that result in an adverse immune response when a subject is exposed to the antigen. In several embodiments, the adverse immune response could be a result of ingestion of the antigen, e.g., orally, nasally, or via some other mucosal route. These routes could be the case, for example, with food antigens. In some embodiments, the antigen may be purposefully administered to a subject, for example, with the administration of a therapeutic composition to treat a disease or condition that the subject is affected by. In still additional embodiments, the antigen may be produced by the subject, e.g., an autoimmune antigen. For example, in several embodiments, the antigen comprises a foreign transplant antigen against which transplant recipients develop an unwanted immune response or a tolerogenic portion thereof. In several embodiments, the antigen comprises a foreign food, animal, plant or environmental antigen against which patients develop an unwanted immune response or a tolerogenic portion thereof. In several embodiments, the antigen comprises a foreign therapeutic agent against which patients develop an unwanted immune response or a tolerogenic portion thereof. In several embodiments, the antigen comprises a synthetic autoantigen against the endogenous version of which patients develop an unwanted immune response or a tolerogenic portion thereof.

In further detail to the above, there are provided in several embodiments, compounds where the antigen is a food antigen. In some such embodiments, the antigen is one or more of conarachin (Ara h 1), allergen II (Ara h 2), arachis agglutinin, conglutin (Ara h 6), a-lactalbumin (ALA), lactotransferrin, Pen a 1 allergen (Pen a 1), allergen Pen m 2 (Pen m 2), tropomyosin fast isoform, high molecular weight glutenin, low molecular weight glutenin, alpha-gliadin, gamma-gliadin, omega-gliadin, hordein, secalin, and avenin. Fragments of any of these antigens and/or mimotopes of any of these antigens are also used, in several embodiments. In several embodiments, the antigen is selected from the group consisting of gluten, high molecular weight glutenin, low molecular weight glutenin, alpha-gliadin, gamma-gliadin, omega-gliadin, hordein, secalin, and avenin and fragments thereof. In several embodiments, the antigen is selected from the group consisting of gluten, high molecular weight glutenin, low molecular weight glutenin, alpha-gliadin, gamma-gliadin, and omega-gliadin and fragments thereof. In several embodiments, the antigen is gluten or fragment thereof. In several embodiments, the antigen is gliadin or fragment thereof.

In several embodiments, there are provided compounds where the antigen is a therapeutic agent. In several embodiments, the antigen is selected from the group consisting of Factor VII, Factor VIII, Factor IX, asparaginase, uricase, adeno-associated viruses (AAV), and fragments of any one thereof. In several embodiments, the antigen is a therapeutic agent selected from the group consisting of Factor VII and Factor IX and fragments thereof. In several embodiments, the antigen is a therapeutic agent selected from the group consisting of Factor VIII or fragment thereof. In several embodiments, when the antigen is a therapeutic agent, the compound can be used in the treatment, prevention, reduction, or otherwise amelioration of an immune response developed against a therapeutic agent for hemophilia. As discussed herein, mimotopes of any antigenic portion of the antigens above can be used in several embodiments.

In several embodiments, the antigen comprises asparaginase or a fragment thereof. In several embodiments, the antigen comprises uricase or a fragment thereof. In several such embodiments, the compound can be used in the treatment, prevention, reduction, or otherwise amelioration of an immune response developed against an anti-neoplastic agent. As discussed herein, mimotopes of any antigenic portion of the antigens above can be used in several embodiments.

In several embodiments, the antigen is associated with an autoimmune disease. For example, in several embodiments, the associated autoimmune disease is one or more of type 1 diabetes, multiple sclerosis, rheumatoid arthritis, vitiligo, uveitis, pemphigus vulgaris, celiac disease, myasthenia gravis, and neuromyelitis optica.

In several embodiments, the autoimmune disease is type 1 diabetes and the antigen comprises insulin or a fragment thereof. In several embodiments, the autoimmune disease is type 1 diabetes and the antigen comprises proinsulin or a fragment thereof. In several embodiments, the autoimmune disease is type 1 diabetes and the antigen comprises preproinsulin or a fragment thereof. As discussed herein, mimotopes of any antigenic portion of the antigens above can be used in several embodiments. In several embodiments, combinations of these antigens can be incorporated into the tolerogenic compound which may aid in reducing immune responses to autoantigens at multiple points along the insulin pathway.

In several embodiments, the autoimmune disease is multiple sclerosis and the antigen comprises myelin basic protein or a fragment thereof. In several embodiments, the autoimmune disease is multiple sclerosis and the antigen comprises myelin oligodendrocyte glycoprotein or a fragment thereof. In several embodiments, the autoimmune disease is multiple sclerosis and the antigen comprises proteolipid protein or a fragment thereof. As discussed herein, mimotopes of any antigenic portion of the antigens above can be used in several embodiments. In several embodiments, combinations of these antigens can be incorporated into the tolerogenic compound (e.g., a mixture of antigens or fragments of MOG, MBP and/or PLP) which may aid in reducing immune responses to autoantigens at multiple points along the pathways that control myelination or myelin repair.

As discussed herein, mimotopes of any antigenic portion of the autoantigens above (or otherwise disclosed herein) can be used in several embodiments.

In several embodiments, the pharmaceutically acceptable composition consists of, or consists essentially of a compound wherein the antigen is a food antigen, therapeutic agent, an autoantigen, or fragment thereof, an optional linker, and a liver targeting moiety as disclosed herein.

The antigen can be a complete protein, a portion of a complete protein, a peptide, or the like, and can be derivatized for conjugation, e.g. with a linker. In some embodiments, the antigen can be a variant and/or can contain conservative substitutions, particularly maintaining sequence identity, and/or can be desialylated (or otherwise modified), relative to what is found naturally.

In some embodiments, the antigen comprises a food antigen. In some embodiments, the food antigen is selected from conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), a-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1IS globulin (Sin a 2), lipid transfer protein (Sin a 3), profilin (Sin a 4), profilin (Api g 4), high molecular weight glycoprotein (Api g 5), tropomyosin (Pen a 1), arginine kinase (Pen m 2), tropomyosin fast isoform, high molecular weight glutenin, low molecular weight glutenin, alpha-, gamma- and omega-gliadin, hordein, secalin, avenin, pathogenesis-related protein from strawberries (Fra a 1), profilin (Mus a 1), a portion of any of said antigens, and a mimetic of any of said antigens.

In some embodiments, the food antigen is selected from the group consisting of gluten, high molecular weight glutenin, low molecular weight glutenin, alpha-gliadin, gamma-gliadin, omega-gliadin, hordein, secalin, and avenin, a portion of any of said antigens, and a mimetic of any of said antigens. In some embodiments, the food antigen is selected from gluten, high molecular weight glutenin, low molecular weight glutenin, alpha-gliadin, gamma-gliadin, and omega-gliadin, a portion of any of said antigens, and a mimetic of any of said antigens. In some embodiments, the food antigen is gluten or a portion or mimetic thereof. In general, these antigens may be associated with gluten intolerance, gluten-sensitive enteropathy, and/or celiac disease. In some embodiments, the antigen is associated with the HLA-DQ2 serotype group.

In celiac disease, main antigens include, but are not limited to, tissue transglutaminase and the natural and deamidated forms of gluten or gluten-like proteins, such as alpha-, gamma-, and omega-gliadin, glutenin, hordein, secalin, and avenin. Those skilled in the art will appreciate, for example, that an antigen associated with gluten intolerance may be converted to be more immunogenic in the body through deamidation by tissue glutaminase converting the antigen's glutamines to glutamic acid. Thus, while an antigen associated with gluten intolerance may be originally considered a foreign food antigen, it may also be considered an autoantigen after modification by the body.

In some embodiments, sequences for wheat gluten proteins and other proteins associated with gluten intolerance and/or celiac disease are generally known in the art, for example, from Bromilow et al. “A curated gluten protein sequence database to support development of proteomics methods for determination of gluten in gluten-free foods” J. Proteomics (2017) 163:67-75, which is hereby expressly incorporated by reference in its entirety.

In some embodiments, peptides or epitopes useful in the tolerogenic compounds disclosed herein for use in preventing unwanted immune response against proteins involved in gluten intolerance and/or celiac disease include some or all of the following sequences, either individually or in combination:

HLA-DQ-2.5 relevant, Alpha-gliadin “33-mer” native:

(SEQ ID NO: 40)
LQLQPFPQPQLPYPQPQLPYPQPQLPYPQPQPF

HLA-DQ-2.5 relevant, Alpha-gliadin “33-mer” deamidated:

(SEQ ID NO: 41)
LQLQPFPQPELPYPQPELPYPQPELPYPQPQPF

HLA-DQ-8 relevant, Alpha-gliadin:

(SEQ ID NO: 42)
QQYPSGQGSFQPSQQNPQ

HLA-DQ-8 relevant, Omega-gliadin (wheat, U5UA46):

(SEQ ID NO: 43)
QPFPQPEQPFPW

Alpha-gliadin “15-mer” fragment:

(SEQ ID NO: 44)
ELQPFPQPELPYPQP

Gliadin with linker:

(SEQ ID NO: 45)
GGGPQPQPFPSQQPY

Gliadin with linker and cysteine conjugation moiety:

(SEQ ID NO: 46)
GCRGGGPQPQPFPSQQPY

Gliadin extended:

(SEQ ID NO: 47)
PQPQPFPSQQPYLQLQPFPQPQLPYPQPQLPYPQPQLPYPQPQPF

Gliadin extended with cysteine conjugation moiety:

(SEQ ID NO: 48)
GCGPQPQPFPSQQPYLQLQPFPQPQLPYPQPQLPYPQPQLPYPQPQPF

Gliadin deamidated extended:

(SEQ ID NO: 49)
PQPQPFPSQQPYLQLQPFPQPELPYPQPELPYPQPELPYPQPQPF

Gliadin deamidated extended:

(SEQ ID NO: 50)
GCGPQPQPFPSQQPYLQLQPFPQPELPYPQPELPYPQPELPYPQPQPF

DQ-8, Alpha-gliadin:

(SEQ ID NO: 51)
FQQPQQQYPSGEGSFQPSQENPQAQ

DQ-8, Alpha-gliadin with cysteine conjugation moiety:

(SEQ ID NO: 52)
GCFQQPQQQYPSGEGSFQPSQENPQAQ

DQ-8, Alpha-gliadin extended:

(SEQ ID NO: 53)
FQQPQQQYPSGEGSFQPSQENPQAQGSVQPQQLPQFEEIRN

DQ-8, Alpha-gliadin extended with cysteine conjugation moiety:

(SEQ ID NO: 54)
GCFQQPQQQYPSGEGSFQPSQENPQAQGSVQPQQLPQFEEIRN

For any of the peptide or epitope sequences provided herein for use as a tolerogen (e.g. SEQ ID NO: 40-54, or a fragment thereof), embodiments may also include sequences having at least 80%, 85%, 90%, 95%, 96%, 97%, 98%, 99% or more sequence identity, which are envisioned to have the same or similar efficacy. Some of the sequences provided herein may include a cysteine conjugation moiety for chemical conjugation. Alternative moieties for chemical conjugation are also envisioned. In alternative embodiments, the sequences provided herein may be fused to an antibody sequence without an additional conjugation moiety, for example, by recombinant cloning techniques.

In some embodiments, the antigen is a foreign antigen associated with an animal, plant, or environmental allergens, toxins, or irritants. Some non-limiting examples include antigens, allergen, toxins, or irritants from cat, mouse, dog, horse, bee, dust, fungus, tree, and plants, including but not limited to:
weeds, (including ragweed allergens Amb a 1, 2, 3, 5, and 6, and Amb t 5; pigweed Che a 2 and 5; and other weed allergens Par j 1, 2, and 3, and Par o 1);
grass (including major allergens Cyn d 1, 7, and 12; Dac g 1, 2, and 5; Hol l 1.01203; Lol p 1, 2, 3, 5, and 11; Mer a 1; Pha a 1; Poap 1 and 5);
pollen from ragweed and other weeds (including curly dock, lambs quarters, pigweed, plantain, sheep sorrel, and sagebrush), grass (including Bermuda, Johnson, Kentucky, Orchard, Sweet vernal, and Timothy grass), and trees (including catalpa, elm, hickory, olive, pecan, sycamore, and walnut);
dust (including major allergens from species Dermatophagoides pteronyssinus, such as Der p 1, 2, 3, 4, 5, 6, 7, 8, 9, 10, 11, 14, 15, 18, 20, 21, and 23; from species Dermatophagoides farina, such as Der f 1, 2, 3, 6, 7, 10, 11, 13, 14, 15, 16, 18, 22, and 24; from species Blomia tropicalis such as Blo t 1, 2, 3, 4, 5, 6, 10, 11, 12, 13, 19, and 21; also allergens Eur m 2 from Euroglyphus maynei, Tyr p 13 from Tyrophagus putrescentiae, and allergens Bla g 1, 2, and 4; Per a 1, 3, and 7 from cockroach);
pets (including cats, dogs, rodents, and farm animals; major cat allergens include Fel d 1 through 8, cat IgA, BLa g 2, and cat albumin; major dog allergens include Can f 1 through 6, and dog albumin);
bee stings, including major allergens Api m 1 through 12; and
fungus, including allergens derived from, species of Aspergillus and Penicillium, as well as the species Alternaria alternata, Davidiella tassiana, and Trichophyton rubrum.

In some embodiments, the antigen comprises an autoantigen. In some embodiments, the autoantigen is selected from thyroglobulin, thyroperoxidase, thyroid-stimulating hormone receptor, glutamic acid decarboxylase (GAD), 21OH hydroxylase, 17OH hydroxylase, H+/K+ ATPase, intrinsic factor, transglutaminase, tyrosinase, tyrosinase-related protein-2, myelin basic protein, myelin oligodendrocyte glycoprotein, proteolipid protein, desmogleins, desmoglien-1, desmoglein-3, desmoglien-4, alpha-synuclein, acetylcholine receptor, 2-oxoacid dehydrogenase complexes, insulin, proinsulin, preproinsulin, insulinoma-associated protein 2 (IA-2), insulinoma-associated protein 213 (IA-213), ICA69, ICA12 (SOX-13), carboxypeptidase H, imogen 38, GLIMA 38, chromogranin-A, HSP-60, carboxypeptidase E, peripherin, glucose transporter 2, hepatocarcinoma-intestine-pancreas/pancreatic associated protein, S100B, glial fibrillary acidic protein, regenerating gene II, pancreatic duodenal homeobox 1, dystrophia myotonica protein kinase (DMPK), islet-specific glucose-6-phosphatase catalytic subunit-related protein, SST G-protein coupled receptors 1-5, myeloperoxidase (MPO), proteinase-3/myeloblastin, and a fragment or portion of any of said antigens, and a mimetic of any of said antigens. In some embodiments, the antigen comprises an antigen associated with an autoimmune disease. In some embodiments, the autoimmune disease is selected from the group consisting of type 1 diabetes, multiple sclerosis, rheumatoid arthritis, vitiligo, uveitis, pemphigus vulgaris, neuromyelitis optica, Goodpasture's Disease, Parkinson's disease, myasthenia gravis, celiac disease, primary biliary cholangitis, Sjogren's syndrome, autoimmune hepatitis, myocarditis, inflammatory cardiomyopathy, and anti-neutrophil cytoplasmic antibody-associated vasculitis.

In several embodiments, the antigen to which tolerance is desired is a viral antigen, for example a viral antigen derived from a therapeutic viral vector, such as an adeno-associated viral vector (AAV). In several embodiments, the antigen to which tolerance is desired comprises is or is an immunogenic fragment derived from the AAV serotype 2 capsid protein 1 (SEQ ID NO: 141). In several embodiments, the antigen to which tolerance is desired comprises is or is an immunogenic fragment derived from the AAV serotype 2 capsid protein 2 (SEQ ID NO: 142). In several embodiments, the antigen to which tolerance is desired comprises is or is an immunogenic fragment derived from the AAV serotype 2 capsid protein 3 (SEQ ID NO: 143). In several embodiments, the antigen to which tolerance is desired comprises is or is an immunogenic fragment derived from the AAV serotype 9 capsid protein 1 (SEQ ID NO: 144). In several embodiments, the antigen to which tolerance is desired comprises is or is an immunogenic fragment derived from the AAV serotype 9 capsid protein 2 (SEQ ID NO: 145). In several embodiments, the antigen to which tolerance is desired comprises is or is an immunogenic fragment derived from the AAV serotype 9 capsid protein 3 (SEQ ID NO: 146).

MART1 (Melanoma antigen recognized by T cells 1, Melan-A), including an exogenously obtained form useful in the compositions of the disclosure, has the following sequence (Uniprot #Q16655):

(SEQ ID NO: 147)
MPREDAHFIYGYPKKGHGHSYTTAEEAAGIGILTVILGVLLLIGC
WYCRRRNGYRALMDKSLHVGTQCALTRRCPQEGFDHRDSKVSLQE
KNCEPWPNAPPAYEKLSAEQSPPPYSP.

Tyrosinase, including an exogenously obtained form useful in the compositions of the disclosure, has the following sequence (Uniprot #P14679):

(SEQ ID NO: 148)
MLLAVLYCLLWSFQTSAGHFPRACVSSKNLMEKECCPPWSGDRSP
CGQLSGRGSCQNILLSNAPLGPQFPFTGVDDRESWPSVFYNRTCQ
CSGNFMGFNCGNCKFGFWGPNCTERRLLVRRNIFDLSAPEKDKFF
AYLTLAKHTISSDYVIPIGTYGQMKNGSTPMFNDINIYDLFVWMH
YYVSMDALLGGSEIWRDIDFAHEAPAFLPWHRLFLLRWEQEIQKL
TGDENFTIPYWDWRDAEKCDICTDEYMGGQHPTNPNLLSPASFFS
SWQIVCSRLEEYNSHQSLCNGTPEGPLRRNPGNHDKSRTPRLPSS
ADVEFCLSLTQYESGSMDKAANFSFRNTLEGFASPLTGIADASQS
SMHNALHIYMNGTMSQVQGSANDPIFLLHHAFVDSIFEQWLRRHR
PLQEVYPEANAPIGHNRESYMVPFIPLYRNGDFFISSKDLGYDYS
YLQDSDPDSFQDYIKSYLEQASRIWSWLLGAAMVGAVLTALLAGL
VSLLCRHKRKQLPEEKQPLLMEKEDYHSLYQSHL.

Melanocyte protein PMEL (gp100), including an exogenously obtained form useful in the compositions of the disclosure, has the following sequence (Uniprot #P40967):

(SEQ ID NO: 149)
MDLVLKRCLLHLAVIGALLAVGATKVPRNQDWLGVSRQLRTKAWN
RQLYPEWTEAQRLDCWRGGQVSLKVSNDGPTLIGANASFSIALNF
PGSQKVLPDGQVIWVNNTIINGSQVWGGQPVYPQETDDACIFPDG
GPCPSGSWSQKRSFVYVWKTWGQYWQVLGGPVSGLSIGTGRAMLG
THTMEVTVYHRRGSRSYVPLAHSSSAFTITDQVPFSVSVSQLRAL
DGGNKHFLRNQPLTFALQLHDPSGYLAEADLSYTWDFGDSSGTLI
SRALVVTHTYLEPGPVTAQVVLQAAIPLTSCGSSPVPGTTDGHRP
TAEAPNTTAGQVPTTEVVGTTPGQAPTAEPSGTTSVQVPTTEVIS
TAPVQMPTAESTGMTPEKVPVSEVMGTTLAEMSTPEATGMTPAEV
SIVVLSGTTAAQVTTTEWVETTARELPIPEPEGPDASSIMSTESI
TGSLGPLLDGTATLRLVKRQVPLDCVLYRYGSFSVTLDIVQGIES
AEILQAVPSGEGDAFELTVSCQGGLPKEACMEISSPGCQPPAQRL
CQPVLPSPACQLVLHQILKGGSGTYCLNVSLADTNSLAVVSTQLI
MPGQEAGLGQVPLIVGILLVLMAVVLASLIYRRRLMKQDFSVPQL
PHSSSHWLRLPRIFCSCPIGENSPLLSGQQV.

Aquaporin-4, including an exogenously obtained form useful in the compositions of the disclosure, has the following sequence (Uniprot #P55087):

(SEQ ID NO: 150)
MSDRPTARRWGKCGPLCTRENIMVAFKGVWTQAFWKAVTAEFLAM
LIFVLLSLGSTINWGGTEKPLPVDMVLISLCFGLSIATMVQCFGH
ISGGHINPAVTVAMVCTRKISIAKSVFYIAAQCLGAIIGAGILYL
VTPPSVVGGLGVTMVHGNLTAGHGLLVELIITFQLVFTIFASCDS
KRTDVTGSIALAIGFSVAIGHLFAINYTGASMNPARSFGPAVIMG
NWENHWIYWVGPIIGAVLAGGLYEYVFCPDVEFKRRFKEAFSKAA
QQTKGSYMEVEDNRSQVETDDLILKPGWHVIDVDRGEEKKGKDQS
GEVLSSV.

In uveitis, main antigens include Retinal S-antigen or “S-arrestin” and interphotoreceptor retinoid binding protein (IRBP) or retinol-binding protein 3.

S-arrestin, including an exogenously obtained form useful in the compositions of the disclosure, has the following sequence (Uniprot #P10523):

(SEQ ID NO: 151)
MAASGKTSKSEPNHVIFKKISRDKSVTIYLGNRDYIDHVSQVQPV
DGVVLVDPDLVKGKKVYVTLTCAFRYGQEDIDVIGLTFRRDLYFS
RVQVYPPVGAASTPTKLQESLLKKLGSNTYPFLLTFPDYLPCSVM
LQPAPQDSGKSCGVDFEVKAFATDSTDAEEDKIPKKSSVRLLIRK
VQHAPLEMGPQPRAEAAWQFFMSDKPLHLAVSLNKEIYFHGEPIP
VTVTVINNTEKTVKKIKAFVEQVANVVLYSSDYYVKPVAMEEAQE
KVPPNSTLTKTLTLLPLLANNRERRGIALDGKIKHEDTNLASSTI
IKEGIDRTVLGILVSYQIKVKLTVSGFLGELTSSEVATEVPFRLM
HPQPEDPAKESYQDANLVFEEFARHNLKDAGEAEEGKRDKNDVDE.

IRBP, including an exogenously obtained form useful in the compositions of the disclosure, has the following sequence (Uniprot #P10745):

(SEQ ID NO: 152)
MMREWVLLMSVLLCGLAGPTHLFQPSLVLDMAKVLLDNYCFPENL
LGMQEAIQQAIKSHEILSISDPQTLASVLTAGVQSSLNDPRLVIS
YEPSTPEPPPQVPALTSLSEEELLAWLQRGLRHEVLEGNVGYLRV
DSVPGQEVLSMMGEFLVAHVWGNLMGTSALVLDLRHCTGGQVSGI
PYIISYLHPGNTILHVDTIYNRPSNTTTEIWTLPQVLGERYGADK
DWVLTSSQTRGVAEDIAHILKQMRRAIVVGERTGGGALDLRKLRI
GESDFFFTVPVSRSLGPLGGGSQTWEGSGVLPCVGTPAEQALEKA
LAILTLRSALPGWVHCLQEVLKDYYTLVDRVPTLLQHLASMDFST
VVSEEDLVTKLNAGLQAASEDPRLLVRAIGPTETPSWPAPDAAAE
DSPGVAPELPEDEAIRQALVDSVFQVSVLPGNVGYLRFDSFADAS
VLGVLAPYVLRQVWEPLQDTEHLIMDLRHNPGGPSSAVPLLLSYF
QGPEAGPVHLFTTYDRRTNITQEHFSHMELPGPRYSTQRGVYLLT
SHRTATAAEEFAFLMQSLGWATLVGEITAGNLLHTRTVPLLDTPE
GSLALTVPVLTFIDNHGEAWLGGGVVPDAIVLAEEALDKAQEVLE
FHQSLGALVEGTGHLLEAHYARPEVVGQTSALLRAKLAQGAYRTA
VDLESLASQLTADLQEVSGDHRLLVFHSPGELVVEEAPPPPPAVP
SPEELTYLIEALFKTEVLPGQLGYLRFDAMAELETVKAVGPQLVR
LVWQQLVDTAALVIDLRYNPGSYSTAIPLLCSYFFEAEPRQHLYS
VFDRATSKVTEVWTLPQVAGQRYGSHKDLYILMSHTSGSAAEAFA
HTMQDLQRATVIGEPTAGGALSVGIYQVGSSPLYASMPTQMAMSA
TTGKAWDLAGVEPDITVPMSEALSIAQDIVALRAKVPTVLQTAGK
LVADNYASAELGAKMATKLSGLQSRYSRVTSEVALAEILGADLQM
LSGDPHLKAAHIPENAKDRIPGIVPMQIPSPEVFEELIKFSFHTN
VLEDNIGYLRFDMFGDGELLTQVSRLLVEHIWKKIMHTDAMIIDM
RFNIGGPTSSIPILCSYFFDEGPPVLLDKIYSRPDDSVSELWTHA
QWGERYGSKKSMVILTSSVTAGTAEEFTYIMKRLGRALVIGEVTS
GGCQPPQTYHVDDTNLYLTIPTARSVGASDGSSWEGVGVTPHVVV
PAEEALARAKEMLQHNQLRVKRSPGLQDHL.

In some embodiments, the autoimmune disease is type 1 diabetes. In some embodiments, the antigen comprises insulin, proinsulin, preproinsulin, glutamic acid decarboxylase-65 (GAD-65), GAD-67, glucose-6-phosphatase 2, islet-specific glucose-6-phosphatase catalytic subunit-related protein (IGRP), insulinoma-associated protein 2 (IA-02), insulinoma-associated protein 2B (IA-2β), ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, and SST G-protein coupled receptors 1-5, or a fragment, portion, or mimetic thereof. In some embodiments, combinations of these antigens can be incorporated into the tolerogenic compound, which may result in a synergistic effect in reducing immune response to autoantigens at multiple points along the insulin pathway. It should be noted that recombinant forms of insulin and derivatives thereof, which are therapeutically used for the treatment of diabetes, is included in some embodiments of the disclosure. It should be noted that insulin is an example of an antigen that can be characterized both as an autoantigen and a therapeutic protein antigen. For example, rHu Insulin and bovine insulin are therapeutic protein antigens (that are the subject of unwanted immune attack), whereas endogenous human insulin is an autoantigen (that is the subject of an unwanted immune attack). Because endogenous human insulin is not available to be employed in a pharmaceutical composition, a recombinant form is employed in certain embodiments of the compositions of the disclosure.

Human insulin, including an exogenously obtained form useful in the compositions of the disclosure, has the following sequence (Uniprot #P01308):

(SEQ ID NO: 153)
MALWMRLLPLLALLALWGPDPAAAFVNQHLCGSHLVEALYLVCGE
RGFFYTPKTRREAEDLQVGQVELGGGPGAGSLQPLALEGSLQKRG
IVEQCCTSICSLYQLENYCN.

GAD-65, including an exogenously obtained form useful in the compositions of the disclosure, has the following sequence (Uniprot #Q05329):

(SEQ ID NO: 154)
MASPGSGFWSFGSEDGSGDSENPGTARAWCQVAQKFTGGIGNKLC
ALLYGDAEKPAESGGSQPPRAAARKAACACDQKPCSCSKVDVNYA
FLHATDLLPACDGERPTLAFLQDVMNILLQYVVKSFDRSTKVIDF
HYPNELLQEYNWELADQPQNLEEILMHCQTTLKYAIKTGHPRYFN
QLSTGLDMVGLAADWLTSTANTNMFTYEIAPVFVLLEYVTLKKMR
EIIGWPGGSGDGIFSPGGAISNMYAMMIARFKMFPEVKEKGMAAL
PRLIAFTSEHSHFSLKKGAAALGIGTDSVILIKCDERGKMIPSDL
ERRILEAKQKGFVPFLVSATAGTTVYGAFDPLLAVADICKKYKIW
MHVDAAWGGGLLMSRKHKWKLSGVERANSVTWNPHKMMGVPLQCS
ALLVREEGLMQNCNQMHASYLFQQDKHYDLSYDTGDKALQCGRHV
DVFKLWLMWRAKGTTGFEAHVDKCLELAEYLYNIIKNREGYEMVF
DGKPQHTNVCFWYIPPSLRTLEDNEERMSRLSKVAPVIKARMMEY
GTTMVSYQPLGDKVNFFRMVISNPAATHQDIDFLIEEIERLGQDL.

IGRP, including an exogenously obtained form useful in the compositions of the disclosure, has the following sequence (Uniprot #QN9QR9):

(SEQ ID NO: 155)
MDFLHRNGVLIIQHLQKDYRAYYTFLNFMSNVGDPRNIFFIYFPL
CFQFNQTVGTKMIWVAVIGDWLNLIFKWILFGHRPYWWVQETQIY
PNHSSPCLEQFPTTCETGPGSPSGHAMGASCVWYVMVTAALSHTV
CGMDKFSITLHRLTWSFLWSVFWLIQISVCISRVFIATHFPHQVI
LGVIGGMLVAEAFEHTPGIQTASLGTYLKTNLFLFLFAVGFYLLL
RVLNIDLLWSVPIAKKWCANPDWIHIDTTPFAGLVRNLGVLFGLG
FAINSEMFLLSCRGGNNYTLSFRLLCALTSLTILQLYHFLQIPTH
EEHLFYVLSFCKSASIPLTWVAFIPYSVHMLMKQSGKKSQ.

In several embodiments, human proinsulin, including an exogenously obtained form useful in the tolerogenic compositions of the disclosure, has the following sequence:

(SEQ ID NO: 156)
FVNQHLCGSHLVEALYLVCGERGFFYTPKTRREAEDLQVGQVELG
GGPGAGSLQPLALEGSLQKRGIVEQCCTSICSLYQLENYCN.

In some embodiments, peptides or epitopes useful in the tolerogenic compounds disclosed herein for use in preventing unwanted immune response against proteins involved in the insulin pathway and/or for treating type 1 diabetes include some or all of the following sequences, either individually or in combination:

Human Proinsulin 1-70:
(SEQ ID NO: 55)
FVNQHLCGSHLVEALYLVCGERGFFYTPKTRREAEDLQVGQ
VELGGGPGAGSLQPLALEGSLQKRGIVEQ;
Human Proinsulin 9-70:
(SEQ ID NO: 56)
SHLVEALYLVCGERGFFYTPKTRREAEDLQVGQVELGGGPGA
GSLQPLALEGSLQKRGIVEQ;
Human Proinsulin 9-38:
(SEQ ID NO: 57)
SHLVEALYLVCGERGFFYTPKTRREAEDLQ;
Human Proinsulin 1-38:
(SEQ ID NO: 58)
FVNQHLCGSHLVEALYLVCGERGFFYTPKTRREAEDLQ;
Human Proinsulin 9-23:
(SEQ ID NO: 59)
SHLVEALYLVCGERG;
Human Proinsulin 45-71 (C13-A6):
(SEQ ID NO: 60)
GGGPGAGSLQPLALEGSLQKRGIVEQC;
Human Proinsulin C24-A1:
(SEQ ID NO: 61)
LALEGSLQKRG;
Human Proinsulin C19-A3:
(SEQ ID NO: 62)
GSLQPLALEGSLQKRGIV;
Human Proinsulin C13-32:
(SEQ ID NO: 63)
GGGPGAGSLQPLALEGSLQK;
Human Proinsulin B9-C4:
(SEQ ID NO: 64)
SHLVEALYLVCGERGFFYTPKTRREAED;
Human Proinsulin C22-A5:
(SEQ ID NO: 65)
QPLALEGSLQKRGIVEQ;
Human IA-2 654-674:
(SEQ ID NO: 66)
AEGPPEPSRVSSVSSQFSDAAQASPSSHSSTPSWCEEPA
Human IA-2 718-782:
(SEQ ID NO: 67)
AYQAEPNTCATAQGEGNIKKNRHPDFLPYDHARIKLKVES
SPSRSDYINASPIIEHDPRMPAYIA;
Human IA-2 785-819:
(SEQ ID NO: 68)
GPLSHTIADFWQMVWESGCTVIVMLTPLVEDGVKQ;
Human IA-2 828-883:
(SEQ ID NO: 69)
GASLYHVYEVNLVSEHIWCEDFLVRSFYLKNVQTQETRT
LTQFHFLSWPAEGTPAS;
Human IA-2 943-979:
(SEQ ID NO: 70)
EHVRDQRPGLVRSKDQFEFALTAVAEEVNAILKALPQCG.

For any of the peptide or epitope sequences provided herein for use as a tolerogen (e.g. SEQ ID NO: 55-70, or a fragment thereof), embodiments may also include sequences having at least 80%, 85%, 90%, 95%, 96%, 97%, 98%, 99% or more sequence identity, which envisioned to have the same or similar efficacy. It is envisioned that the sequences may also include a cysteine conjugation moiety or any other alternative moieties for chemical conjugation. In alternative embodiments, the sequences provided herein may be fused to an antibody sequence without an additional conjugation moiety, for example, by recombinant cloning techniques.

In some embodiments, the autoimmune disease is multiple sclerosis. In some embodiments, the antigen comprises myelin basic protein, myelin oligodendrocyte glycoprotein, proteolipid protein, or any fragment, portion, or mimetic thereof. In some embodiments, combinations of these antigens can be incorporated into the tolerogenic compound, which may result in a synergistic effect in reducing immune responses to autoantigens at multiple points along the pathological pathway to promote resolution of disease.

In some embodiments, peptides or epitopes useful in the tolerogenic compounds disclosed herein for use in preventing unwanted immune response against proteins involved in myelination, myelin repair, and/or for treating multiple sclerosis. In multiple sclerosis, main antigens include, but are not limited to, myelin basic protein (“MBP”), myelin oligodendrocyte glycoprotein (“MOG”) and myelin proteolipid protein (“PLP”).

MBP, including an exogenously obtained form useful in the compositions of the disclosure, has the following sequence (Uniprot #P02686):

(SEQ ID NO: 157)
MGNHAGKRELNAEKASTNSETNRGESEKKRNLGELSRTTSEDNEV
FGEADANQNNGTSSQDTAVTDSKRTADPKNAWQDAHPADPGSRPH
LIRLFSRDAPGREDNTFKDRPSESDELQTIQEDSAATSESLDVMA
SQKRPSQRHGSKYLATASTMDHARHGFLPRHRDTGILDSIGRFFG
GDRGAPKRGSGKDSHHPARTAHYGSLPQKSHGRTQDENPWVHFFK
NIVTPRTPPPSQGKGRGLSLSRFSWGAEGQRPGFGYGGRASDYKS
AHKGFKGVDAQGTLSKIFKLGGRDSRSGSPMARR

MOG, including an exogenously obtained form useful in the compositions of the disclosure, has the following sequence (Uniprot #Q16653):

(SEQ ID NO: 158)
MASLSRPSLPSCLCSFLLLLLLQVSSSYAGQFRVIGPRHPIRALV
GDEVELPCRISPGKNATGMEVGWYRPPFSRWHLYRNGKDQDGDQA
PEYRGRTELLKDAIGEGKVTLRIRNVRFSDEGGFTCFFRDHSYQE
EAAMELKVEDPFYWVSPGVLVLLAVLPVLLLQITVGLIFLCLQYR
LRGKLRAEIENLHRTFDPHFLRVPCWKITLFVIVPVLGPLVALII
CYNWLHRRLAGQFLEELRNPF

PLP, including an exogenously obtained form useful in the compositions of the disclosure, has the following sequence (Uniprot #P60201):

(SEQ ID NO: 159)
MGLLECCARCLVGAPFASLVATGLCFFGVALFCGCGHEALTGTEK
LIETYFSKNYQDYEYLINVIHAFQYVIYGTASFFFLYGALLLAEG
FYTTGAVRQIFGDYKTTICGKGLSATVTGGQKGRGSRGQHQAHSL
ERVCHCLGKWLGHPDKFVGITYALTWWWLLVFACSAVPVYIYFNT
WTTCQSIAFPSKTSASIGSLCADARMYGVLPWNAFPGKVCGSNLL
SICKTAEFQMTFHLFIAAFVGAAATLVSLLTFMIAATYNFAVLKL
MGRGTKF

In several embodiments, the peptides or epitopes useful in the tolerogenic compounds disclosed herein include some or all of the following sequences derived from myelin basic protein (MBP), myelin oligodendrocyte glycoprotein (MOG), or myelin proteolipid protein (PLP), either individually or in combination:

MBP 1-39:
(SEQ ID NO: 71)
GCASQKRPSQRHGSKYLATASTMDHARHGFLPRHRDTGILDS;
MBP 13-32:
(SEQ ID NO: 72)
KYLATASTMDHARHGFLPRH;
MBP 76-106:
(SEQ ID NO: 73)
SHGRTQDENPVVHFFKNIVTPRTPPPSQGKGCG;
MBP 83-99 (altered peptide ligand):
(SEQ ID NO: 74)
ENPWHFFKNIVTPRTP;
MBP 102-136:
(SEQ ID NO: 75)
SQGKGRGLSLSRFSWGAEGQRPGFGYGGRASDYKSCG
MBP 111-129:
(SEQ ID NO: 76)
LSRFSWGAEGQRPGFGYGG;
MBP 146-170:
(SEQ ID NO: 77)
AQGTLSKIFKLGGRDSRSGSPMARR;
MBP 146-170 with cysteine conjugation moiety:
(SEQ ID NO: 78)
AQGTLSKIFKLGGRDSRSGSPMARRCG;
MBP 83-99:
(SEQ ID NO: 79)
ENPVVHFFKNIVTPRTP;
MBP 82-98:
(SEQ ID NO: 80)
DENPVVHFFKNIVTPRT;
MBP 82-99:
(SEQ ID NO: 81)
DENPVVHFFKNIVTPRTP;
MBP 82-106:
(SEQ ID NO: 82)
DENPWHFFKNIVTPRTPPPSQGKG;
MBP 87-106:
(SEQ ID NO: 83)
VHFFKNIVTPRTPPPSQGKG;
MBP 131-155:
(SEQ ID NO: 84)
ASDYKSAHKGLKGVDAQGTLSKIFK;
MBP 131-170 with cysteine conjugation moiety:
(SEQ ID NO: 85)
ASDYKSAHKGFKGVDAQGTLSKIFKLGGRDSRSGSPMARRCG;
MBP 76-136:
(SEQ ID NO: 86)
SHGRTQDENPVVHFFKNIVTPRTPPPSQGKGRGLSLSRFSWGAE
GQRPGFGYGGRASDYKSCG
MOG 1-20:
(SEQ ID NO: 87)
GQFRVIGPRHPIRALVGDEV;
MOG 1-27:
(SEQ ID NO: 88)
GQFRVIGPRHPIRALVGDEVELPCRIS;
Alternate MOG 35-55:
(SEQ ID NO: 89)
MEVGWYRPPFSRWHLYRNGK;
MOG 30-60:
(SEQ ID NO: 90)
KNATGMEVGWYRSPFSRVVHLYRNGKDQDAE;
MOG 34-56:
(SEQ ID NO: 91)
GMEVGWYRSPFSRVVHLYRNGKD;
MOG 35-55:
(SEQ ID NO: 92)
MEVGWYRPPFSRVVHLYRNGK;
MOG 35-55 (Mouse):
(SEQ ID NO: 93)
MEVGWYRSPFSRVVHLYRNGK;
MOG 33-62:
(SEQ ID NO: 94)
TGMEVGWYRPPFSRVVHLYRNGKDQDGDQA;
MOG 11-30:
(SEQ ID NO: 95)
PIRALVGDEVELPCRISPGK;
MOG 18-62:
(SEQ ID NO: 96)
DEVELPCRISPGKNATGMEVGWYRPPFSRVVHLYRNGKDQDGDQA;
MOG 21-40:
(SEQ ID NO: 97)
ELPCRISPGKNATGMEVGWY;
MOG 64-86:
(SEQ ID NO: 98)
EYRGRTELLKDAIGEGKVTLRIR;
MOG 1-60:
(SEQ ID NO. 99)
GQFRVIGPRHPIRALVGDEVELPCRISPGKNATGMEV
GWYRPPFSRVVHLYRNGKDQDGD;
MOG 1-62:
(SEQ ID NO. 100)
GQFRVIGPRHPIRALVGDEVELPCRISPGKNATGMEVG
WYRPPFSRVVHLYRNGKDQDGDQA;
PLP 41-58:
(SEQ ID NO: 101)
GTEKLIETYFSKNYQDYE;
PLP 89-106:
(SEQ ID NO: 102)
GFYTTGAVRQIFGDYKTT;
PLP 95-116:
(SEQ ID NO: 103)
AVRQIFGDYKTTICGKGLSATV;
PLP 178-197:
(SEQ ID NO: 104)
NTWTTCQSIAFPSKTSASIG;
PLP 190-209:
(SEQ ID NO: 105)
SKTSASIGSLCADARMYGVL;
and
PLP 139-154:
(SEQ ID NO: 106)
HCLGKWLGHPDKFVGI.

For any of the peptide or epitope sequences provided herein for use as a tolerogen (e.g. SEQ ID NO: 75-106), embodiments may also include sequences having at least 80%, 85%, 90%, 95%, 96%, 97%, 98%, 99% or more sequence identity, which envisioned to have the same or similar efficacy. Some of the sequences provided herein may include a cysteine conjugation moiety for chemical conjugation. Alternative moieties for chemical conjugation are also envisioned. In alternative embodiments, the sequences provided herein may be fused to an antibody sequence without an additional conjugation moiety, for example, by recombinant cloning techniques.

In some embodiments, the antigen is a therapeutic agent. In some embodiments, the antigen is selected from the group consisting of Factor VIII, Factor IX, asparaginase, uricase, adeno-associated viruses (AAV) (i.e. for use in gene therapy), and mimetics, fragments or portions of any of the aforementioned antigens. In some embodiments, the antigen is associated with hemophilia.

In the embodiments where the antigen is a therapeutic protein, peptide, antibody or antibody-like molecule, specific antigens can be selected from: Abatacept, Abciximab, Adalimumab, Adenosine deaminase, Ado-trastuzumab emtansine, Agalsidase alfa, Agalsidase beta, Aldeslukin, Alglucerase, Alglucosidase alfa, α-1-proteinase inhibitor, Anakinra, Anistreplase (anisoylated plasminogen streptokinase activator complex), Antithrombin III, Antithymocyte globulin, Ateplase, Bevacizumab, Bivalirudin, Botulinum toxin type A, Botulinum toxin type B, C1-esterase inhibitor, Canakinumab, Carboxypeptidase G2 (Glucarpidase and Voraxaze), Certolizumab pegol, Cetuximab, Collagenase, Crotalidae immune Fab, Darbepoetin-α, Denosumab, Digoxin immune Fab, Dornase alfa, Eculizumab, Etanercept, Factor Vlla, Factor VIII, Factor IX, Factor XI, Factor XIII, Fibrinogen, Filgrastim, Galsulfase, Golimumab, Histrelin acetate, Hyaluronidase, Idursulphase, Imiglucerase, Infliximab, Insulin [including recombinant human insulin (“rHu insulin”) and bovine insulin], Interferon-α2a, Interferon-α2b, Interferon-β1a, Interferon-β1b, Interferon-γ1b, Ipilimumab, L-arginase, L-asparaginase, L-methionase, Lactase, Laronidase, Lepirudin/hirudin, Mecasermin, Mecasermin rinfabate, Methoxy Natalizumab, Octreotide, Ofatumumab, Oprelvekin, Pancreatic amylase, Pancreatic lipase, Papain, Peg-asparaginase, Peg-doxorubicin HCl, PEG-epoetin-β, Pegfilgrastim, Peg-Interferon-α2a, Peg-Interferon-α2b, Pegloticase, Pegvisomant, Phenylalanine ammonia-lyase (PAL), Protein C, Rasburicase (uricase), Sacrosidase, Salmon calcitonin, Sargramostim, Streptokinase, Tenecteplase, Teriparatide, Tocilizumab (atlizumab), Trastuzumab, Type 1 alpha-interferon, Ustekinumab, vW factor. The therapeutic protein can be obtained from natural sources (e.g., concentrated and purified) or synthesized, e.g., recombinantly, and includes antibody therapeutics that are typically IgG monoclonal or fragments or fusions.

Particular therapeutic protein, peptide, antibody or antibody-like molecules include, but are not limited to, Abciximab, Adalimumab, Agalsidase alfa, Agalsidase beta, Aldeslukin, Alglucosidase alfa, Factor VIII, Factor IX, Infliximab, Insulin (including rHu Insulin), L-asparaginase, Laronidase, Natalizumab, Octreotide, Phenylalanine ammonia-lyase (PAL), or Rasburicase (uricase) and generally IgG monoclonal antibodies in their varying formats.

Some embodiments employ hemostatic agents (e.g., Factor VIII and IX), Insulin (including rHu Insulin), and the therapeutic molecules uricase, phenylalanine ammonia lyase (PAL) and asparaginase (which may be non-human in origin).

In several embodiments, therapeutic agents are delivered through the use of, e.g., a gene therapy vector. In some such embodiments, an immune response may be developed against a portion of such vectors and/or their cargo (e.g., the therapeutic agent). Thus, in several embodiments, the antigen to which tolerance is desired comprises a gene therapy vector, including, but are not limited to: adenoviruses and adeno-associated virus (and corresponding variants-1, -2, -5, -6, -8, -9, and/or other parvoviruses), lentiviruses, and retroviruses.

In some embodiments, for autoimmune diseases of the thyroid, including Hashimoto's thyroiditis and Graves' disease, example antigens for use in the tolerogenic compounds include, but are not limited to, thyroglobulin (TG), thyroid peroxidase (TPO), thyrotropin receptor (TSHR), sodium iodine symporter (NIS), or megalin. In some embodiments, for thyroid-associated ophthalmopathy and dermopathy, insulin-like growth factor 1 receptor is another example antigen. In some embodiments, for hypoparathyroidism, calcium sensitive receptor is another example antigen.

In some embodiments, for Addison's disease, example antigens for use in the tolerogenic compounds include, but are not limited to, 21OH hydroxylase, 17OH hydroxylase, cytochrome P450 side-chain cleavage enzyme (P450scc) and/or P450c21 or P450c17, or adrenocorticotropic hormone receptor (ACTH receptor).

In some embodiments, for premature ovarian failure, example antigens for use in the tolerogenic compounds include, but are not limited to, FSH receptor and α-enolase.

In some embodiments, for autoimmune hypophysitis or pituitary autoimmune disease, example antigens for use in the tolerogenic compounds include, but are not limited to, pituitary gland-specific protein factor (PGSF) 1a and 2, or type 2 iodothyronine deiodinase.

In some embodiments, for rheumatoid arthritis, example antigens for use in the tolerogenic compounds include, but are not limited, to, collagen I, collagen II, immunoglobulin binding protein, double stranded DNA, fibrin, fibrinogen (including fibrinogen alpha, beta, or gamma chains), vimentin, aggrecan, or α-enolase.

An example sequence for collagen II is provided as Uniprot #P02458 (SEQ ID NO: 107). In some embodiments, the tolerogenic compound may comprise amino acids 1236-1249 (SEQ ID NO: 108), 662-678 (SEQ ID NO: 160), 459-478 (SEQ ID NO: 161), 461-473 (SEQ ID NO: 162), 471-485 (SEQ ID NO: 163), 450-470 (SEQ ID NO: 164), 595-603 (SEQ ID NO: 165), 455-474 (SEQ ID NO: 166), 498-511 (SEQ ID NO: 167), 1133-1150 (SEQ ID NO: 168), 1427-1435 (SEQ ID NO: 169), 378-440 (SEQ ID NO: 170), 1350-1362 (SEQ ID NO: 171), 1237-1249 (SEQ ID NO: 172), 511-525 (SEQ ID NO: 173), or 756-764 (SEQ ID NO: 174) of collagen II. In some embodiments, the tolerogenic compound is post-translationally modified, for example, amino acids 461-473 of collagen II are carbamylated at K4 (SEQ ID NO: 272), amino acids 455-474 of collagen II are hydroxylated at P4 and P19 (SEQ ID NO: 273), amino acids 1237-1249 of collagen II are citrullinated at R4 (SEQ ID NO: 274), amino acids 511-525 of collagen II are citrullinated at R5 (SEQ ID NO: 275), or amino acids 756-764 of collagen II are hydroxylated at P6 (SEQ ID NO: 276).

An example sequence for aggrecan is provided as Uniprot #P16112 (SEQ ID NO: 109). In some embodiments, the tolerogenic compound may comprise amino acids 84-103 (SEQ ID NO: 110), 32-64 (SEQ ID NO: 199), 68-82 (SEQ ID NO: 200), 76-110 (SEQ ID NO: 201), 116-130 (SEQ ID NO: 202), 140-169 (SEQ ID NO: 203), 161-177 (SEQ ID NO: 204), 174-188 (SEQ ID NO: 205), 200-215 (SEQ ID NO: 206), 225-244 (SEQ ID NO: 207), 252-269 (SEQ ID NO: 208), 287-314 (SEQ ID NO: 209), 341-355 (SEQ ID NO: 210), 520-539 (SEQ ID NO: 211), 553-570 (SEQ ID NO: 212), 568-583 (SEQ ID NO: 213), 589-601 (SEQ ID NO: 214), 620-636 (SEQ ID NO: 215), 666-674 (SEQ ID NO: 216), 714-725 (SEQ ID NO: 217), 866-879 (SEQ ID NO: 218), 988-998 (SEQ ID NO: 219), 1056-1067 (SEQ ID NO: 220), 1352-1366 (SEQ ID NO: 221), 1645-1658 (SEQ ID NO: 222), 1892-1904 (SEQ ID NO: 223), 1785-1798 (SEQ ID NO: 224), 1805-1819 (SEQ ID NO: 225), 1885-1898 (SEQ ID NO: 226), 1937-1950 (SEQ ID NO: 227), 1961-1974 (SEQ ID NO: 228), 2018-2031 (SEQ ID NO: 229), 2104-2118 (SEQ ID NO: 230), 2189-2201 (SEQ ID NO: 231), 2282-2296 (SEQ ID NO: 232), 2351-2368 (SEQ ID NO: 233), 2507-2521 (SEQ ID NO: 234), 2513-2528 (SEQ ID NO: 235), or 2516-2530 (SEQ ID NO: 236) of aggrecan. In some embodiments, the tolerogenic compound is post-translationally modified, for example, amino acids 161-177 of aggrecan are citrullinated at R3, R10, and R₁₃ (SEQ ID NO: 282), amino acids 200-215 of aggrecan are citrullinated at R11 (SEQ ID NO: 283), amino acids 225-244 of aggrecan are citrullinated at R7 and R12 (SEQ ID NO: 284), amino acids 520-539 of aggrecan are citrullinated at R11 and R16 (SEQ ID NO: 285), amino acids 553-570 of aggrecan are citrullinated at R4 and R9 (SEQ ID NO: 286), amino acids 568-583 of aggrecan are citrullinated at R8 (SEQ ID NO: 287), or amino acids 620-636 of aggrecan are citrullinated at R13 (SEQ ID NO: 288).

An example sequence for fibrinogen alpha chain is provided as Uniprot #P02671 (SEQ ID NO: 111). In some embodiments, the tolerogenic compound may comprise amino acids 78-91 (SEQ ID NO: 112), 717-725 (SEQ ID NO: 113), 51-95 (SEQ ID NO:175), 138-152 (SEQ ID NO: 176), 171-185 (SEQ ID NO: 177), 201-215 (SEQ ID NO: 178), 300-314 (SEQ ID NO: 179), 347-361 (SEQ ID NO: 180), 363-377 (SEQ ID NO: 181), 420-434 (SEQ ID NO: 182), 438-452 (SEQ ID NO: 183), 456-470 (SEQ ID NO: 184), 542-556 (SEQ ID NO: 185), 717-725 (SEQ ID NO: 186), or 737-751 (SEQ ID NO: 187) of fibrinogen alpha chain. In some embodiments, the tolerogenic compound is post-translationally modified, for example, amino acids 138-152 of fibrinogen alpha chain is citrullinated at R6 (SEQ ID NO: 277), amino acids 456-470 of fibrinogen alpha chain is citrullinated at R3 and R4 (SEQ ID NO: 278), amino acids 717-725 of fibrinogen alpha chain is citrullinated at R4 (SEQ ID NO: 279), or amino acids 737-751 of fibrinogen alpha chain is citrullinated at R7 (SEQ ID NO: 280).

An example sequence for fibrinogen beta chain is provided as Uniprot #P02675 (SEQ ID NO: 188). In some embodiments, the tolerogenic compound may comprise amino acids 433-411 (SEQ ID NO: 189) or 69-81 (SEQ ID NO: 190) of fibrinogen beta chain.

An example sequence for vimentin is provided as Uniprot #P08670 (SEQ ID NO: 114). In some embodiments, the tolerogenic compound may comprise amino acids 66-78 (SEQ ID NO: 115), 447-455 (SEQ ID NO: 116), 26-49 (SEQ ID NO: 191), 51-88 (SEQ ID NO: 192), 116-122 (SEQ ID NO: 193), 130-138 (SEQ ID NO: 194), 226-242 (SEQ ID NO: 195), 371-387 (SEQ ID NO: 196), 415-443 (SEQ ID NO: 197), or 447-455 (SEQ ID NO: 198) of vimentin. In some embodiments, the tolerogenic compound is post-translationally modified, for example, amino acids 447-455 of vimentin is citrullinated at R4 (SEQ ID NO: 281).

An example sequence for alpha enolase is provided as Uniprot #P06733 (SEQ ID NO: 237). In some embodiments, the tolerogenic compound may comprise amino acids 4-40 (SEQ ID NO: 238) or 326-340 (SEQ ID NO: 239) of alpha enolase.

In some embodiments, for autoimmune gastritis, example antigens for use in the tolerogenic compounds include, but are not limited to, H+/K+ ATPase.

In some embodiments, for pernicious anemia, example antigens for use in the tolerogenic compounds include, but are not limited to, intrinsic factor.

In some embodiments, for vitiligo, example antigens for use in the tolerogenic compounds include, but are not limited to, tyrosinase or tyrosinase related protein 1 and 2.

In some embodiments, for myasthenia gravis, example antigens for use in the tolerogenic compounds include, but are not limited to, nicotinic acetylcholine receptor (AChR), muscle-specific kinase (MuSK), or lipoprotein receptor-related protein 4 (LRP4).

In some embodiments, for pemphigus vulgaris and variants, example antigens for use in the tolerogenic compounds include, but are not limited to, desmogelin-1, desmoglein-3, desmoglein-4, desmocollin 1, plectin, plakoglobin, periplakin, desmoplakin I and II, envoplakin, and acetylcholine receptor.

In some embodiments, for bullous pemphigoid, example antigens for use in the tolerogenic compounds include, but are not limited to, BP180, BP230, plectin, or laminin 5.

In some embodiments, for dermatitis herpetiformis (Duhring's disease), example antigens for use in the tolerogenic compounds include, but are not limited to, endomysium or tissue transglutaminase.

In some embodiments, for epidermolysis bullosa acquisita, example antigens for use in the tolerogenic compounds include, but are not limited to, collagen VII.

In some embodiments, for systemic sclerosis, example antigens for use in the tolerogenic compounds include, but are not limited to, matrix metalloproteinase 1, matrix metalloproteinase 3, heat-shock protein 47, fibrillin 1, PDGF receptor, Scl-70, U1 snRNP, Th/To, Ku, Jo1, NAG-2, centromere proteins, topoisomerase I, nucleolar proteins, RNA polymerase I, II, or III, PM-Scl, fibrillarin, or B23.

In some embodiments, for mixed connective tissue disease, example antigens for use in the tolerogenic compounds include, but are not limited to, U1 snRNP.

In some embodiments, for Sjogren's syndrome, example antigens for use in the tolerogenic compounds include, but are not limited to, nuclear antigens SS-A (Ro) and SS-B (La), fodrin, poly (ADP-ribose) polymerase, topoisomerase, muscarinic receptors, and Fc-gamma receptor IIIb. An example sequence for SS-A is provided as Uniprot #P19474 (SEQ ID NO: 117).

An example sequence for SS-B is provided as Uniprot #P05455 (SEQ ID NO: 118). In some embodiments, the tolerogenic compound may comprise amino acids 19-33 (SEQ ID NO: 240), 46-72 (SEQ ID NO: 241), 151-180 (SEQ ID NO: 242), 211-228 (SEQ ID NO: 243), 244-258 (SEQ ID NO: 244), or 304-329 (SEQ ID NO: 245) of SS-B.

In some embodiments, for systemic lupus erythematosus, example antigens for use in the tolerogenic compounds include, but are not limited to, “Smith antigen”, SS-A, high mobility group box 1 (HMGB1), nucleosomes, histone proteins, and double stranded DNA.

In some embodiments, for Goodpasture's syndrome, example antigens for use in the tolerogenic compounds include, but are not limited to, glomerular basement membrane proteins or collagen IV.

In some embodiments, for rheumatic heart disease, autoimmune myocarditis, viral myocarditis, and/or inflammatory cardiomyopathy, example antigens for use in the tolerogenic compounds include, but are not limited to, cardiac myosin and myosin 6. An example sequence for cardiac myosin is provided as Uniprot #P13533 (SEQ ID NO: 119).

An example sequence for myosin 6 is provided as Uniprot #P12883 (SEQ ID NO: 251). In some embodiments, the tolerogenic compound may comprise amino acids 1906-1923 (SEQ ID NO: 252), 1828-1845 (SEQ ID NO: 253), 1411-1493 (SEQ ID NO: 254), 1554-1584 (SEQ ID NO: 255), 1170-1181 (SEQ ID NO: 256), 1619-1636 (SEQ ID NO: 257), 1671-1714 (SEQ ID NO: 258), 450-464 (SEQ ID NO: 259), 1801-1819 (SEQ ID NO: 260), 1333-1363 (SEQ ID NO: 261), 1867-1897 (SEQ ID NO: 262), 1749-1814 (SEQ ID NO: 263), 1142-1156 (SEQ ID NO: 264), 1372-1389 (SEQ ID NO: 265), 1710-1727 (SEQ ID NO: 266), 1645-1662 (SEQ ID NO: 267), 689-703 (SEQ ID NO: 268), 1593-1610 (SEQ ID NO: 269), or 111-119 (SEQ ID NO: 270) of myosin 6.

In some embodiments, for autoimmune polyendocrine syndrome type I, example antigens for use in the tolerogenic compounds include, but are not limited to, aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and P4502A6, SOX-9, SOX-10, calcium-sensing receptor protein, or type 1 interferons.

In some embodiments, for neuromyelitis optica, example antigens for use in the tolerogenic compounds include, but are not limited to, aquaporin 4.

In some embodiments, for uveitis, example antigens for use in the tolerogenic compounds include, but are not limited to, retinal S-antigen, S-arrestin, interphotoreceptor retinoid binding protein (IRBP), or retinol-binding protein 3.

In some embodiments, for Parkinson's disease, example antigens for use in the tolerogenic compounds include, but are not limited to, alpha-synuclein. An example sequence for alpha-synuclein is provided as Uniprot #P37840 (SEQ ID NO: 120). In some embodiments, the tolerogenic compound may comprise amino acids 2-23 (SEQ ID NO: 246), 32-46 (SEQ ID NO: 247), 56-85 (SEQ ID NO: 248), 81-110 (SEQ ID NO: 249) or 116-140 (SEQ ID NO: 250) of alpha-synuclein.

In some embodiments, for anti-neutrophil cytoplasmic antibody-associated vasculitis (ANCA-V, ANCA-vasculitis), example antigens for use in the tolerogenic compounds include, but are not limited to, myeloperoxidase and proteinase-3/myeloblastin. An example sequence for myeloperoxidase is provided as Uniprot #P05164 (SEQ ID NO: 121). In some embodiments, the tolerogenic compound may comprise amino acids 447-461 (SEQ ID NO: 122), 435-454 (SEQ ID NO: 123), 409-423 (SEQ ID NO: 124), 409-474 (SEQ ID NO: 125), 279-341 (SEQ ID NO: 126), 341-409 (SEQ ID NO: 127), or 598-745 (SEQ ID NO: 128) of myeloperoxidase. An example sequence for proteinase-3 is provided as Uniprot #P24158 (SEQ ID NO: 129).

In some embodiments, for primary biliary cholangitis, example antigens for use in the tolerogenic compounds include, but are not limited to, pyruvate dehydrogenase complex E2 subunit (PDC-E2). An example sequence for PDC-E2 is provided as Uniprot #P10515 (SEQ ID NO: 130). In some embodiments, the tolerogenic compound may comprise amino acids 163-176 (SEQ ID NO: 131), 159-167 (SEQ ID NO: 132), or 159-176 (SEQ ID NO: 271) of PDC-E2.

In some embodiments, for autoimmune hepatitis, example antigens for use in the tolerogenic compounds include, but are not limited to, cytochrome P450 2D6 (CYP2D6). An example sequence for CYP2D6 is provided as Uniprot #P10635 (SEQ ID NO: 133). In some embodiments, the tolerogenic compound may comprise amino acids 245-254 (SEQ ID NO: 134), 217-260 (SEQ ID NO: 135), 305-348 (SEQ ID NO: 136), 193-212 (SEQ ID NO: 137), 305-325 (SEQ ID NO: 138), 313-322 (SEQ ID NO: 139), or 393-412 (SEQ ID NO: 140) of CYP2D6.

In some embodiments, the antigen comprises an alloantigen. In some embodiments, the alloantigen is selected from the group consisting of subunits of the MHC class I and MHC class II haplotype proteins and their complexes with the antigens they present (for example, donor/recipient differences identified in tissue cross-matching), and minor blood group antigens RhCE, Kell, Kidd, Duffy, Ss, Diego, and MNSs (including single nucleotide polymorphisms), and mimetics, fragments, or portions thereof. In some embodiments, the antigen is associated with graft-vs-host disease, transplant rejection, or autoimmune aplastic anemia. Such compositions can be prepared individually for a given donor/recipient pair.

Additional allergens with more description, as well as nomenclature as standardized by the WHO/IUIS Allergen Nomenclature Sub-Committee used herein (e.g., “Aca f 2”) may be found on the world wide web at www.allergen.org.

In some embodiments, the antigen of the tolerogenic composition may comprise one or more of any of the antigen sequences disclosed herein, such as any one or more of SEQ ID NO: 40-288, or fragment thereof, or any other antigen generally known in the art.

Also disclosed herein are compositions comprising any one or more of the tolerogenic compounds disclosed herein and a pharmaceutically acceptable excipient.

The pharmaceutical compositions may be manufactured in a manner that is itself known, e.g., by means of conventional mixing, dissolving, granulating, dragee-making, levigating, emulsifying, encapsulating, entrapping, or tableting processes. Additionally, the active ingredients are contained in an amount effective to achieve its intended purpose. Many of the compounds used in the pharmaceutical combinations disclosed herein may be provided as salts with pharmaceutically compatible counterions.

The compositions may, if desired, be presented in a dispenser device which may contain one or more unit dosage forms containing the active ingredient. The dispenser device may be accompanied by instructions for administration. The dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Methods of Inducing Tolerance

Also disclosed herein are methods of inducing tolerance to an antigen to which a subject is capable of developing an unwanted immune response. In some embodiments, the methods comprise administering any one or more of the tolerogenic compounds or compositions disclosed herein. In some embodiments, the tolerogenic compound or the composition is administered prior to the subject being exposed to the antigen, after the subject has been exposed to the antigen, or both. In some embodiments, the unwanted immune response is associated with an allergy towards a food, animal, plant, or environmental allergen, an autoimmune disease, a therapeutic agent, or graft-vs-host disease, or other diseases and disorders associated with any of the antigens disclosed herein.

Also disclosed herein are the tolerogenic compounds and compositions disclosed herein for use in inducing tolerance in a subject to an antigen to which the subject is capable of developing an unwanted immune response. In some embodiments, the unwanted immune response is associated with an allergy towards a food, animal, plant, or environmental allergen, an autoimmune disease, a therapeutic agent, or graft-vs-host disease, or other diseases or disorders associated with any of the antigens disclosed herein.

Also disclosed herein are the tolerogenic compounds and compositions disclosed herein for use in the manufacture of a medicament.

In some embodiments, the compounds or compositions provided herein are used in the treatment, prevention, reduction, or otherwise alter an immune response to an antigen. In some embodiments, the immune response has occurred or is occurring in an ongoing manner. In some embodiments, the treatment and use of the compounds or compositions are used in a prophylactic manner. In some embodiments, the administration to a subject is performed before, after, or before and after exposure of the subject to an antigen. In some embodiments, administration prior to exposure serves a prophylactic effect to essentially avoid or significantly reduce the unwanted immune response.

Administration of the compositions can be via a variety of methods, including, but not limited to parenteral, intravenous, infusion, intramuscular, oral, rectal, pulmonary, topical, aerosol, transdermal, intradermal, or other administration route. In several embodiments, the compositions are delivered in a therapeutically effective amount, for example, by a systemic or local route (e.g., intravenous, intraarterially, locally, intramuscular, subcutaneous, intramedullary, intrathecal, intraventricular, intraperitoneal, intranasal, intraocular etc.). Administration can be performed at time points that are less frequent or that are substantially equal to yearly, monthly, daily, weekly, multiple times per day, or on an as needed basis (e.g., prior to an anticipated exposure).

In some embodiments, the compounds or compositions are administered in an amount sufficient to result in induction of clonal deletion and/or anergy of T cells that are specific for the antigen of interest. In some embodiments, the compounds or compositions are configured to target hepatocytes and/or LSEC. In some embodiments, the compounds or compositions are configured to induce expansion of certain populations, or sub-populations, of regulatory T cells, such as CD4⁺CD25⁺FOXP3⁺ regulatory T cells. In some embodiments, the compounds or compositions are administered in an amount effective to reduce a concentration of antibodies that are causatively involved with any of the disease or disorders disclosed herein, such as graft-vs-host disease, transplant rejection, immune response against a therapeutic agent, autoimmune disease, hypersensitivity, and/or allergy, in the blood of a patient by at least 10%, 20%, 30%, 40%, or 50%, or any percentage within a range defined by any two of the aforementioned reductions.

The compounds or compositions described herein can be administered to a patient per se, or in combinations where they are mixed with other active ingredients, such as in combination therapy.

In several embodiments, the compounds or compositions is provided as a unit dose. In several embodiments, the methods and uses disclosed herein includes administering a unit dose to a patient or subject. In several embodiments, the unit dose includes 1 μg/kg to 10 mg/kg of an tolerogenic compound to body weight of a subject. In several embodiments, the tolerogenic compound to body weight per administration in a single dose is equal to or less than: about 10 μg/kg, about 50 μg/kg, about 75 μg/kg, about 0.1 mg/kg, about 0.2 mg/kg, about 0.5 mg/kg, about 0.75 mg/kg, about 1.0 mg/kg, about 1.5 mg/kg, about 2.0 mg/kg, about 2.5 mg/kg, about 4.0 mg/kg, about 5.0 mg/kg, 10.0 mg/kg, or ranges spanning and/or including the aforementioned values. In some embodiments, the quantity of tolerogenic compound that is administered is at a unit dose that is less than or equal less or equal to 10, 5, 2, 1, 0.5, 0.1, 0.05, 0.01, 0.005, 0.001, or 0.0005 mg per kg of bodyweight. In several embodiments, a dosing regimen is provided for, wherein a subject receives at least one dose of a composition according to embodiments disclosed herein. In several embodiments, the subject receives at least two, at least three, at least four, at least five, or more doses of a composition according to embodiments disclosed herein. In several embodiments, a given subsequent dose is provided at a concentration that is less than or equal to the prior dose. For example, when receiving a second dose, if the concentration of the first dose was 0.5 mg/kg, the second dose may be provided at about 0.25 mg/kg. In additional embodiments, the doses are held constant over time. Depending on the severity of the underlying immune response (or potential immune response), the doses optionally escalate over time.

### NON-LIMITING EMBODIMENTS

1. An asialoglycoprotein receptor 1 (ASGR1) binding polypeptide comprising a heavy chain variable (VH) region comprising a first heavy chain complementarity determining region (HCDR1), a second heavy chain complementarity determining region HCDR2, and a third heavy chain complementarity determining region HCDR3;
wherein the HCDR1 comprises the sequence of SEQ ID NO: 9, 297, 329,361, 393, 425, 457, 489, 521, or 553;
wherein the HCDR2 comprises the sequence of SEQ ID NO: 10, 298, 330, 362, 394, 426, 458, 490, 522, or 554; and
wherein the HCDR3 comprises the sequence of SEQ ID NO: 11, 299, 331, 363, 395, 427, 459, 491, 523, or 555.

2. The ASGR1 binding polypeptide of embodiment 1, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 15, 303, 335, 367, 399, 431, 463, 495, 527, or 559.

3. The ASGR1 binding polypeptide of embodiment 1, further comprising a light chain variable region (VL) comprising a first light chain complementarity determining region (LCDR1), a second light chain complementarity determining region (LCDR2), and a third light chain complementarity determining region (LCDR3);
wherein the LCDR1 comprises the sequence of SEQ ID NO: 25, 313, 345, 377, 409, 441, 473, 505, 537, or 569;
wherein the LCDR2 comprises the sequence of SEQ ID NO: 26, 314, 346, 378, 410, 442, 474, 506, 538, or 570; and
wherein the LCDR3 comprises the sequence of SEQ ID NO: 27, 315, 347, 379, 411, 443, 475, 507, 539, or 571.

4. The ASGR1 binding polypeptide of embodiment 3, wherein the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 31, 319, 351, 383, 415, 447, 479, 511, 543, or 575.

5. The ASGR1 binding polypeptide of embodiment 1, wherein the heavy chain variable region comprises the sequence of SEQ ID NO: 15, 303, 335, 367, 399, 431, 463, 495, 527, or 559 and the light chain variable region comprises the sequence of SEQ ID NO: 31, 319, 351, 383, 415, 447, 479, 511, 543, or 575.

6. The ASGR1 binding polypeptide of embodiment 1, wherein the ASGR1 binding polypeptide is an antibody, an Fab′ fragment, an F(ab′)2 fragment, a domain antibody (dAb), or an scFv.

7. The ASGR1 binding polypeptide of embodiment 1, wherein the ASGR1 binding polypeptide comprises an Fc domain, optionally wherein the Fc domain is silenced.

8. A polynucleotide encoding for the ASGR1 binding polypeptide of embodiment 1, the polynucleotide comprising one or more of the sequences of SEQ ID NO: 12-14, 300-302, 332-334, 364-366, 396-398, 428-430, 460-462, 492-494, 524-526, or 556-558.

9. The polynucleotide of embodiment 8, wherein the polynucleotide comprises the sequence of SEQ ID NO: 16, 304, 336, 368, 400, 432, 464, 496, 528, or 560.

10. A polynucleotide encoding for the ASGR1 binding polypeptide of embodiment 3, the polynucleotide comprising one or more of the sequences of SEQ ID NO: 25-27, 313-315, 345-347, 377-379, 409-411, 441-443, 473-475, 505-507, 537-539, or 569-571.

11. The polynucleotide of embodiment 10, wherein the polynucleotide comprises the sequence of SEQ ID NO: 32, 320, 352, 384, 416, 448, 480, 512, 544, or 576.

12. The polynucleotide of embodiment 11, wherein the polynucleotide further comprises the sequence of SEQ ID NO: 16, 304, 336, 368, 400, 432, 464, 496, 528, or 560.

13. A tolerogenic compound comprising an ASGR1 binding polypeptide according to any one of Embodiments 1 to 12, wherein the ASGR1 binding polypeptide is conjugated or fused to an antigen to which tolerance is desired.

14. The tolerogenic compound of Embodiment 13, wherein the ASGR1 binding polypeptide and the antigen are conjugated or fused with a linker, optionally wherein the linker is a polypeptide linker or a chemical conjugation linker.

15. The tolerogenic compound of embodiment 14, wherein the linker is a cleavable linker.

16. The tolerogenic compound of embodiment 15, wherein the linker comprises glycine and/or serine, optionally wherein the linker comprises the sequence of SEQ ID NO: 37.

17. The tolerogenic compound of embodiment 13, wherein the antigen is conjugated or fused to the N-terminus or C-terminus of the ASGR1 binding polypeptide.

18. The tolerogenic compound of embodiment 13, wherein the antigen comprises a food antigen.

19. The tolerogenic compound of Embodiment 18, wherein the food antigen is associated with celiac disease.

20. The tolerogenic compound of embodiment 19, wherein the antigen comprises a sequence having at least 80%, 85%, 90%, 95%, 96%, 97%, 98%, 99%, or 100% sequence identity to SEQ ID NO: 41.

21. The tolerogenic compound of embodiment 19, wherein the antigen comprises SEQ ID NO: 41.

22. The tolerogenic compound of any one of embodiments 13-19, wherein the food antigen is selected from conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), a-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1IS globulin (Sin a 2), lipid transfer protein (Sin a 3), profilin (Sin a 4), profilin (Api g 4), high molecular weight glycoprotein (Api g 5), tropomyosin (Pen a 1), arginine kinase (Pen m 2), tropomyosin fast isoform, high molecular weight glutenin, low molecular weight glutenin, alpha-, gamma- and omega-gliadin, hordein, secalin, avenin, pathogenesis-related protein from strawberries (Fra a 1), profilin (Mus a 1), a portion of any of said antigens, and a mimetic of any of said antigens.

23. The tolerogenic compound of any one of embodiments 13-19 or 22, wherein the food antigen is selected from the group consisting of high molecular weight glutenin, low molecular weight glutenin, alpha-, gamma- and omega-gliadin, hordein, secalin, avenin, a portion of any of said antigens, and a mimetic of any of said antigens.

24. The tolerogenic compound of any one of embodiments 13-19, wherein the antigen comprises a sequence having at least 80%, 85%, 90%, 95%, 96%, 97%, 98%, 99%, or 100% sequence identity to any one of SEQ ID NO: 40-54, or a fragment thereof.

25. The tolerogenic compound of any one of embodiments 13-17, wherein the antigen comprises an autoantigen.

26. The tolerogenic compound of embodiment 25, wherein the autoantigen is selected from thyroglobulin, thyroperoxidase, thyroid-stimulating hormone receptor, glutamic acid decarboxylase (GAD), 210H hydroxylase, 17OH hydroxylase, H+/K+ ATPase, intrinsic factor, transglutaminase, tyrosinase, tyrosinase-related protein-2, myelin basic protein, proteolipid protein, desmogleins, acetylcholine receptor, 2-oxoacid dehydrogenase complexes, insulin, proinsulin, preproinsulin, insulinoma-associated protein 2 (IA-2), insulinoma-associated protein 213 (IA-213), ICA69, ICA12 (SOX-13), carboxypeptidase H, imogen 38, GLIMA 38, chromogranin-A, HSP-60, carboxypeptidase E, peripherin, glucose transporter 2, hepatocarcinoma-intestine-pancreas/pancreatic associated protein, S100ß, glial fibrillary acidic protein, regenerating gene II, pancreatic duodenal homeobox 1, dystrophia myotonica protein kinase (DMPK), islet-specific glucose-6-phosphatase catalytic subunit-related protein, SST G-protein coupled receptors 1-5, myeloperoxidase (MPO), proteinase-3/myeloblastin, and a portion of any of said antigens, and a mimetic of any of said antigens.

27. The tolerogenic compound of any one of embodiments 13-17 or 25-26, wherein the antigen comprises an antigen associated with an autoimmune disease.

28. The tolerogenic compound of embodiment 27, wherein the autoimmune disease is selected from the group consisting of multiple sclerosis, type 1 diabetes, rheumatoid arthritis, vitiligo, uveitis, pemphigus vulgaris, neuromyelitis optica, Goodpasture's Disease, Parkinson's disease, myasthenia gravis, celiac disease, primary biliary cholangitis, Sjogren's syndrome, autoimmune hepatitis, myocarditis, inflammatory cardiomyopathy, and anti-neutrophil cytoplasmic antibody-associated vasculitis.

29. The tolerogenic compound of embodiment 27 or 28, wherein the autoimmune disease is multiple sclerosis.

30. The tolerogenic compound of embodiment 29, wherein the antigen comprises a sequence having at least 80%, 85%, 90%, 95%, 96%, 97%, 98%, 99%, or 100% sequence identity to any one of SEQ ID NO: 100, 71-99, 101-106 or 157-159, or a fragment thereof.

31. The tolerogenic compound of embodiment 27 or 28, wherein the autoimmune disease is type 1 diabetes.

32. The tolerogenic compound of embodiment 31, wherein the antigen comprises a sequence having at least 80%, 85%, 90%, 95%, 96%, 97%, 98%, 99%, or 100% sequence identity to any one of SEQ ID NO: 55-70 or 153-156, or a fragment thereof.

33. A tolerogenic compound comprising an ASGR1 binding polypeptide conjugated or fused to an antigen to which tolerance is desired, wherein the ASGR1 binding polypeptide comprises a heavy chain variable region comprising an HCDR1, HCDR2, and HCDR3;

wherein the HCDR1 comprises the sequence of SEQ ID NO: 9, 297, 329,361, 393, 425, 457, 489, 521, or 553;

wherein the HCDR2 comprises the sequence of SEQ ID NO: 10, 298, 330, 362, 394, 426, 458, 490, 522, or 554; and

wherein the HCDR3 comprises the sequence of SEQ ID NO: 11, 299, 331, 363, 395, 427, 459, 491, 523, or 555.

34. The tolerogenic compound of embodiment 33, wherein the heavy chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 15, 303, 335, 367, 399, 431, 463, 495, 527, or 559.

35. The tolerogenic compound of embodiment 33 or 34, wherein the ASGR1 binding polypeptide further comprises a light chain variable region comprising an LCDR1, LCDR2, and LCDR3;
wherein the LCDR1 comprises the sequence of SEQ ID NO: 25, 313, 345, 377, 409, 441, 473, 505, 537, or 569;
wherein the LCDR2 comprises the sequence of SEQ ID NO: 26, 314, 346, 378, 410, 442, 474, 506, 538, or 570; and
wherein the LCDR3 comprises the sequence of SEQ ID NO: 27, 315, 347, 379, 411, 443, 475, 507, 539, or 571.

36. The tolerogenic compound of embodiment 35, the light chain variable region comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 31, 319, 351, 383, 415, 447, 479, 511, 543, or 575.

37. The tolerogenic compound of any one of embodiments 33-36, wherein the heavy chain variable region comprises the sequence of SEQ ID NO: 15, 303, 335, 367, 399, 431, 463, 495, 527, or 559 and the light chain variable region comprises the sequence of SEQ ID NO: 31, 319, 351, 383, 415, 447, 479, 511, 543, or 575.

38. The tolerogenic compound of any one of embodiments 33-37, wherein the ASGR1 binding polypeptide is an antibody, an Fab′ fragment, an F(ab′)2 fragment, a domain antibody (dAb), or an scFv.

39. The tolerogenic compound of any one of embodiments 33-38, wherein the ASGR1 binding polypeptide comprises an Fc domain, optionally wherein the Fc domain is silenced.

40. The tolerogenic compound of embodiment 34-39, wherein the antigen is a polypeptide.

41. The tolerogenic compound of any one of embodiments 33-40, wherein the ASGR1 binding polypeptide and the antigen are conjugated or fused with a linker, optionally wherein the linker is a polypeptide linker or a chemical conjugation linker.

42. The tolerogenic compound of embodiment 41, wherein the linker is a cleavable linker.

43. The tolerogenic compound of embodiment 41, wherein the linker comprises glycine and/or serine, optionally wherein the linker comprises the sequence of SEQ ID NO: 37.

44. The tolerogenic compound of any one of embodiments 33-43, wherein the antigen is conjugated or fused to the N-terminus or C-terminus of the ASGR1 binding polypeptide.

45. The tolerogenic compound of any one of embodiments 33-44, wherein the ASGR1 binding polypeptide comprises an Fc domain, optionally wherein the Fc domain is silenced, and the antigen is conjugated or fused to the Fc domain, optionally wherein the antigen is conjugated or fused to the C-terminus of the Fc domain.

46. The tolerogenic compound of any one of embodiments 33-35, wherein the antigen comprises a food antigen.

47. The tolerogenic compound of embodiment 46, wherein the food antigen is selected from conarachin (Ara h 1), allergen II (Ara h 2), arachis agglutinin, conglutin (Ara h 6), 31 kda major allergen/disease resistance protein homolog (Mal d 2), lipid transfer protein precursor (Mal d 3), major allergen Mal d 1.03D (Mal d 1), a-lactalbumin (ALA), lactotransferrin, actinidin (Act c 1, Act d 1), phytocystatin, thaumatin-like protein (Act d 2), kiwellin (Act d 5), ovomucoid, ovalbumin, ovotransferrin, and lysozyme, livetin, apovitillin, vosvetin, 2S albumin (Sin a 1), 1IS globulin (Sin a 2), lipid transfer protein (Sin a 3), profilin (Sin a 4), profilin (Api g 4), high molecular weight glycoprotein (Api g 5), tropomyosin (Pen a 1), arginine kinase (Pen m 2), tropomyosin fast isoform, high molecular weight glutenin, low molecular weight glutenin, alpha-, gamma- and omega-gliadin, hordein, secalin, avenin, pathogenesis-related protein from strawberries (Fra a 1), profilin (Mus a 1), a portion of any of said antigens, and a mimetic of any of said antigens.

48. The tolerogenic compound of embodiment 46 or 47, wherein the food antigen is selected from the group consisting of high molecular weight glutenin, low molecular weight glutenin, alpha-, gamma- and omega-gliadin, hordein, secalin, avenin, a portion of any of said antigens, and a mimetic of any of said antigens.

49. The tolerogenic compound of any one of embodiments 46-48, wherein the food antigen is associated with celiac disease.

50. The tolerogenic compound of embodiment 49, wherein the antigen comprises a sequence having at least 80%, 85%, 90%, 95%, 96%, 97%, 98%, 99%, or 100% sequence identity to any one of SEQ ID NO: 40-54, or a fragment thereof.

51. The tolerogenic compound of any one of embodiments 33-45, wherein the antigen comprises an autoantigen.

52. The tolerogenic compound of embodiment 51, wherein the autoantigen is selected from thyroglobulin, thyroperoxidase, thyroid-stimulating hormone receptor, glutamic acid decarboxylase (GAD), 21OH hydroxylase, 17OH hydroxylase, H+/K+ ATPase, intrinsic factor, transglutaminase, tyrosinase, tyrosinase-related protein-2, myelin basic protein, proteolipid protein, desmogleins, acetylcholine receptor, 2-oxoacid dehydrogenase complexes, insulin, proinsulin, preproinsulin, insulinoma-associated protein 2 (IA-2), insulinoma-associated protein 213 (IA-213), ICA69, ICA12 (SOX-13), carboxypeptidase H, imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica protein kinase (DMPK), islet-specific glucose-6-phosphatase catalytic subunit-related protein, SST G-protein coupled receptors 1-5, myeloperoxidase (MPO), proteinase-3/myeloblastin, and a portion of any of said antigens, and a mimetic of any of said antigens.

53. The tolerogenic compound of any one of embodiments 33-45 or 51-52, wherein the antigen comprises an antigen associated with an autoimmune disease.

54. The tolerogenic compound of embodiment 53, wherein the autoimmune disease is selected from the group consisting of type 1 diabetes, multiple sclerosis, rheumatoid arthritis, vitiligo, uveitis, pemphigus vulgaris, neuromyelitis optica, Goodpasture's Disease, Parkinson's disease, myasthenia gravis, celiac disease, primary biliary cholangitis, Sjogren's syndrome, autoimmune hepatitis, myocarditis, inflammatory cardiomyopathy, and anti-neutrophil cytoplasmic antibody-associated vasculitis.

55. The tolerogenic compound of embodiment 53 or 54, wherein the autoimmune disease is type 1 diabetes.

56. The tolerogenic compound of embodiment 55, wherein the antigen comprises a sequence having at least 80%, 85%, 90%, 95%, 96%, 97%, 98%, 99%, or 100% sequence identity to any one of SEQ ID NO: 55-70 or 153-156, or a fragment thereof.

57. The tolerogenic compound of embodiment 53 or 54, wherein the autoimmune disease is multiple sclerosis.

58. The tolerogenic compound of embodiment 57, wherein the antigen comprises a sequence having at least 80%, 85%, 90%, 95%, 96%, 97%, 98%, 99%, or 100% sequence identity to any one of SEQ ID NO: 71-106 or 157-159, or a fragment thereof.

59. The tolerogenic compound of embodiment 53 or 54, wherein the autoimmune disease is:
rheumatoid arthritis, wherein the antigen comprises a sequence having at least 80%, 85%, 90%, 95%, 96%, 97%, 98%, 99%, or 100% sequence identity to any one of SEQ ID NO: 107-116, 160-239 or 272-288, or a fragment thereof;
Sjogren's syndrome, wherein the antigen comprises a sequence having at least 80%, 85%, 90%, 95%, 96%, 97%, 98%, 99%, or 100% sequence identity to any one of SEQ ID NO: 117-118 or 240-245, or a fragment thereof;
rheumatic heart disease, autoimmune myocarditis, viral myocarditis, or inflammatory cardiomyopathy, wherein the antigen comprises a sequence having at least 80%, 85%, 90%, 95%, 96%, 97%, 98%, 99%, or 100% sequence identity to any one of SEQ ID NO: 119 or 251-270, or a fragment thereof;
Parkinson's disease, wherein the antigen comprises a sequence having at least 80%, 85%, 90%, 95%, 96%, 97%, 98%, 99%, or 100% sequence identity to any one of SEQ ID NO: 120 or 247-250, or a fragment thereof;
anti-neutrophil cytoplasmic antibody-associated vasculitis (ANCA-vasculitis), wherein the antigen comprises a sequence having at least 80%, 85%, 90%, 95%, 96%, 97%, 98%, 99%, or 100% sequence identity to any one of SEQ ID NO: 121-129, or a fragment thereof;
primary biliary cholangitis, wherein the antigen comprises a sequence having at least 80%, 85%, 90%, 95%, 96%, 97%, 98%, 99%, or 100% sequence identity to any one of SEQ ID NO: 130-132 or 271, or a fragment thereof; or
autoimmune hepatitis, wherein the antigen comprises a sequence having at least 80%, 85%, 90%, 95%, 96%, 97%, 98%, 99%, or 100% sequence identity to any one of SEQ ID NO: 133-140, or a fragment thereof.

60. The tolerogenic compound of any one of embodiments 33-45, wherein the antigen comprises an alloantigen.

61. The tolerogenic compound of embodiment 60, wherein the alloantigen is selected from the group consisting of subunits of the MHC class I and MHC class II haplotype proteins and their complexes with the antigens they present, and minor blood group antigens RhCE, Kell, Kidd, Duffy, Diego, and MNSs.

62. A composition comprising the tolerogenic compound of any one of embodiments 33-61 and a pharmaceutically acceptable excipient.

63. A method of inducing tolerance to an antigen to which a subject is capable of developing an unwanted immune response, comprising administering the tolerogenic compound of any one of embodiments 33-61 or the composition of embodiment 62 to the subject.

64. The method of embodiment 63, wherein the tolerogenic compound or the composition is administered prior to the subject being exposed to the antigen, after the subject has been exposed to the antigen, or both.

65. The method of embodiment 63 or 64, wherein the unwanted immune response is associated with an allergy towards a food, animal, plant, or environmental allergen, an autoimmune disease, a therapeutic agent, or graft-vs-host disease.

66. The tolerogenic compound of any one of embodiments 33-45 or the composition of embodiment 63 for use in inducing tolerance in a subject to an antigen to which the subject is capable of developing an unwanted immune response.

67. The tolerogenic compound or the composition for use of embodiment 66, wherein the unwanted immune response is associated with an allergy towards a food, animal, plant, or environmental allergen, an autoimmune disease, a therapeutic agent, or graft-vs-host disease.

68. The tolerogenic compound of any one of embodiments 33-45 or the composition of embodiment 62 for use in the manufacture of a medicament.

69. The compound of any one of embodiments 33-45 or the composition of embodiment 62 for use in the induction of immune tolerance in a subject in need thereof.

70. A method of inducing tolerance to an antigen for which tolerance is desired, the method comprising:
administering to a subject a compound comprising:
(i) an asialoglycoprotein receptor 1 (ASGR1) binding polypeptide comprising a heavy chain variable region comprising an HCDR1, HCDR2, and HCDR3;
wherein the HCDR1 comprises a sequence having at least 90% identity to SEQ ID NO: 9, 297, 329,361, 393, 425, 457, 489, 521, or 553;
wherein the HCDR2 comprises a sequence having at least 90% identity to SEQ ID NO: 10, 298, 330, 362, 394, 426, 458, 490, 522, or 554; and
wherein the HCDR3 comprises a sequence having at least 90% identity to SEQ ID NO: 11, 299, 331, 363, 395, 427, 459, 491, 523, or 555; and
(ii) an antigen to which tolerance is desired,
wherein the ASGR1 binding polypeptide is conjugated or fused to the antigen to which tolerance is desired.

71. The method of embodiment 70, wherein the ASGR1 binding polypeptide further comprises a light chain variable region comprising an LCDR1, LCDR2, and LCDR3;
wherein the LCDR1 comprises a sequence having at least 90% identity to SEQ ID NO: 25, 313, 345, 377, 409, 441, 473, 505, 537, or 569;
wherein the LCDR2 comprises a sequence having at least 90% identity to SEQ ID NO: 26, 314, 346, 378, 410, 442, 474, 506, 538, or 570; and
wherein the LCDR3 comprises a sequence having at least 90% identity to SEQ ID NO: 27, 315, 347, 379, 411, 443, 475, 507, 539, or 571.

72. The method of embodiment 70 or 71, wherein the antigen to which tolerance is desired is associated with Celiac Disease.

73. The method of embodiment 72, wherein the antigen comprises a sequence having at least 80%, 85%, 90%, 95%, 96%, 97%, 98%, 99%, or 100% sequence identity to any one of SEQ ID NO: 41, 40, 43-54, or a fragment thereof.

74. The method of embodiment 70 or 71, wherein the antigen to which tolerance is desired is associated with MS.

75. The method of embodiment 74, wherein the antigen comprises a sequence having at least 80%, 85%, 90%, 95%, 96%, 97%, 98%, 99%, or 100% sequence identity to any one of SEQ ID NO: 100, 71-99, 101-106 or 157-159, or a fragment thereof.

76. The method of embodiment 70 or 71, wherein the antigen to which tolerance is desired is associated with type 1 diabetes.

77. The method of embodiment 76, wherein the antigen comprises a sequence having at least 80%, 85%, 90%, 95%, 96%, 97%, 98%, 99%, or 100% sequence identity to any one of SEQ ID NO: 55-70 or 153-156, or a fragment thereof.

78. The method of any one of embodiments 70-77, wherein the administering is via an intravenous route.

79. A method of delivering an antigen to liver tissue of a subject, the method comprising:
conjugating or fusing an antigen to an asialoglycoprotein receptor 1 (ASGR1) binding polypeptide, thereby generating an ASGR1 binding polypeptide-antigen complex; and
causing the ASGR1 binding polypeptide-antigen complex to be contacted with liver tissue of a subject in situ,
wherein the contacting allows the ASGR1 binding polypeptide to bind to ASGR1 on the liver tissue, thereby delivering the antigen to liver tissue.

80. A method of delivering an antigen to liver tissue of a subject, the method comprising:
causing an asialoglycoprotein receptor 1 (ASGR1) binding polypeptide, conjugated of fused to an antigen to contact liver tissue of a subject in situ, thereby allowing the ASGR1 binding polypeptide to bind to ASGR1 on the liver tissue,
wherein the ASGR1 binding polypeptide comprises a heavy chain variable region comprising an HCDR1, HCDR2, and HCDR3;
wherein the HCDR1 comprises a sequence having at least 90% identity to SEQ ID NO: 9, 297, 329,361, 393, 425, 457, 489, 521, or 553;
wherein the HCDR2 comprises a sequence having at least 90% identity to SEQ ID NO: 10, 298, 330, 362, 394, 426, 458, 490, 522, or 554; and
wherein the HCDR3 comprises a sequence having at least 90% identity to SEQ ID NO: 11, 299, 331, 363, 395, 427, 459, 491, 523, or 555; and
wherein the ASGR1 binding polypeptide comprises a light chain variable region comprising an LCDR1, LCDR2, and LCDR3;
wherein the LCDR1 comprises a sequence having at least 90% identity to SEQ ID NO: 25, 313, 345, 377, 409, 441, 473, 505, 537, or 569;
wherein the LCDR2 comprises a sequence having at least 90% identity to SEQ ID NO: 26, 314, 346, 378, 410, 442, 474, 506, 538, or 570; and
wherein the LCDR3 comprises a sequence having at least 90% identity to SEQ ID NO: 27, 315, 347, 379, 411, 443, 475, 507, 539, or 571.

81. The method of embodiment 70 or 80, wherein the delivery of the antigen to the liver tissue of the subject in situ induces tolerance in the subject to the antigen.

82. The method of any one of embodiments 80-81 for treatment of an autoimmune disease.

83. The method of any one of embodiments 80-82 for treatment of an allergy.

### EXAMPLES

Additional embodiments are disclosed in further detail in the following examples, which are not in any way intended to limit the scope of the claims. The methods and materials represent non-limiting embodiments of how the compounds disclosed herein can be made, screened, tested or otherwise evaluated.

### Example 1: Materials and Methods

Antibodies were generated against the extracellular domain (ECD) of human ASGR1 (SEQ ID NO: 34). This ECD corresponds to amino acids Q62-L291 of the full length wild-type ASGR1 sequence (SEQ ID NO: 33).

### ASGR1 Preparation

ASGR1 was biotinylated using the EZ-Link Sulfo-NHS-Biotinylation Kit (Thermo Scientific, Cat #21425).

The ASGR1 proteins were concentrated to ˜1 mg/ml and buffer exchanged into PBS before addition of 1:7.5 molar ratio biotinylation reagent. The mixture was held at 4 C overnight prior to another buffer exchange to remove free biotin in the solution. Biotinylation was confirmed through streptavidin sensor binding of the labeled proteins on a ForteBio.

### Naïve Library Selections

Eight naïve human synthetic yeast libraries each of ˜109 diversity were propagated as previously described (see, e.g., Y. Xu et al, PEDS 26(10), 663-70(2013); WO2009036379; WO2010105256; and WO2012009568, each of which is hereby expressly incorporated by reference in its entirety.)

For the first two rounds of selection, a magnetic bead sorting technique utilizing the Miltenyi MACS system was performed, as previously described (see, e.g., Siegel et al, J Immunol Methods 286(1-2), 141-153 (2004), which is hereby expressly incorporated by reference in its entirety.) Briefly, yeast cells (˜1010 cells/library) were incubated with 50 nM biotinylated human ASGR1-His for 30 min at 30° C. in wash buffer (HEPES-buffered saline (HBS)/0.1% bovine serum albumin (BSA)/1 mM CaCl2). After washing once with 40 mL ice-cold wash buffer, the cell pellet was resuspended in 20 mL wash buffer, and Streptavidin MicroBeads (500 μl) were added to the yeast and incubated for 15 min at 4° C. Next the yeast were pelleted, resuspended in 5 mL wash buffer, and loaded onto a Miltenyi LS column. After the 5 mL were loaded, the column was washed 3 times with 3 mL wash buffer. The column was then removed from the magnetic field, and the yeast were eluted with 5 ml of growth media and then grown overnight.

The following rounds of selection were performed using flow cytometry (FACS). Yeast were pelleted, washed three times with wash buffer, and incubated at 30° C. with either 50 nM biotinylated human ASGR1-His, 50 nM biotinylated cyno ASGR1-His, 50 nM biotinylated mouse ASGR1-His, or 50 nM biotinylated rat ASGR1-His in order to obtain species cross-reactivity, or with a polyspecificity reagent (PSR) to remove non-specific antibodies from the selection. For the PSR depletion, the libraries were incubated with a 1:10 dilution of biotinylated PSR reagent as previously described (see, e.g., Y. Xu et al, PEDS 26(10), 663-70 (2013).) Yeast were also incubated with 50 nM biotinylated human ASGR1-His without CaCl₂) to enrich for calcium-dependent antibodies. After the various incubations, yeast were then washed twice with wash buffer and stained with goat F(ab′)2 anti-human kappa-FITC (LC-FITC) diluted 1:100 (Southern Biotech, Cat #2062-02) and either Streptavidin-AF633 (SA-633) diluted 1:500 (Life Technologies, Cat #S21375) or Extravidin-phycoerythrin (EA-PE) diluted 1:50 (Sigma-Aldrich, Cat #E4011), secondary reagents for 15 min at 4° C. After washing twice with ice-cold wash buffer, the cell pellets were resuspended in 0.3 mL wash buffer and transferred to strainer-capped sort tubes. Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast were plated and individual colonies were picked for characterization.

### Light Chain Batch Shuffle Diversification

Heavy chains from naïve output were used to prepare light chain diversification libraries used for additional selection rounds. Selections were performed on these libraries as described above, i.e., with one round of MACS and four rounds of FACS. In the different FACS selection rounds, the libraries were evaluated for, e.g., PSR binding, species cross-reactivity, calcium dependence, and affinity pressure by ASGR1 titration. Sorting was performed in order to obtain a population with the desired characteristics. Individual colonies from each terminal FACS selection round were picked for sequencing and characterization.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 3.5.

### ForteBio Octet KD Measurements

ForteBio affinity measurements were performed on an Octet HTX generally as previously described (see, e.g., Estep et al, Mabs 5(2), 270-278 (2013). Briefly, ForteBio affinity measurements were performed by loading IgGs on-line onto AHC sensors. Sensors were equilibrated off-line in assay buffer for 30 min and then monitored on-line for 60 seconds for baseline establishment. Sensors with loaded IgGs were exposed to 100 nM ASGR1 for 3 minutes, and afterwards were transferred to assay buffer for 3 min for off-rate measurement. All kinetics were analyzed using the 1:1 binding model.

### ForteBio Epitope Binning

Epitope binning was performed using a standard sandwich format cross-blocking assay. Control anti-ASGR1 IgG was loaded onto AHQ sensors and unoccupied Fc-binding sites on the sensor were blocked with an irrelevant human IgG1 antibody. The sensors were then exposed to 100 nM human ASGR1-His followed by a second anti-ASGR1 antibody. Additional binding by the second antibody after ASGR1 association indicates an unoccupied epitope (non-competitor), while no binding indicates epitope blocking (competitor).

### Cell Binding Analysis

Approximately 200,000 cells overexpressing human ASGR1 or parental cells were washed with wash buffer and incubated with 100 μL of 100 nM IgG for 15 minutes at room temperature. Cells were then washed twice with wash buffer and incubated with 100 μL of 1:100 anti-Human IgG R-PE (Southern Biotech Cat #2040-09) for 15 minutes on ice. Cells were then washed twice with wash buffer and analyzed on a FACS Canto II analyzer (BD Biosciences.)

### Example 2: Non-Limiting Examples of Anti-ASGR1 Antibodies

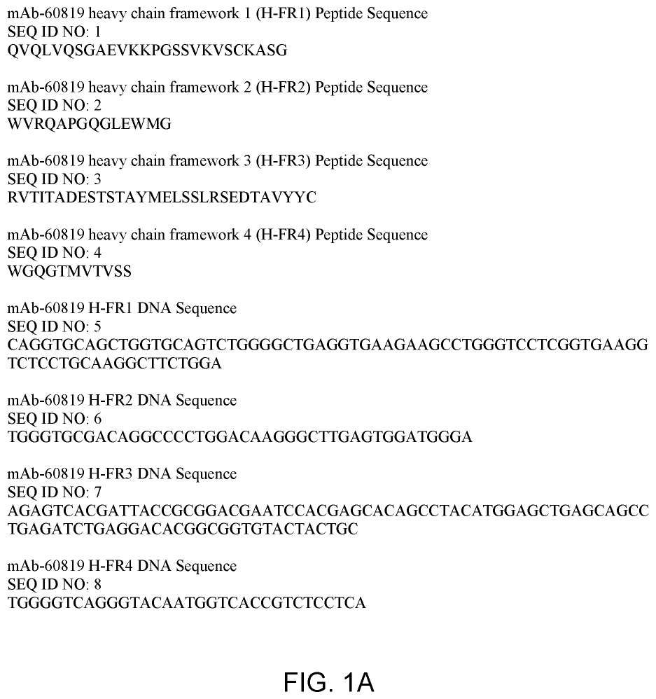

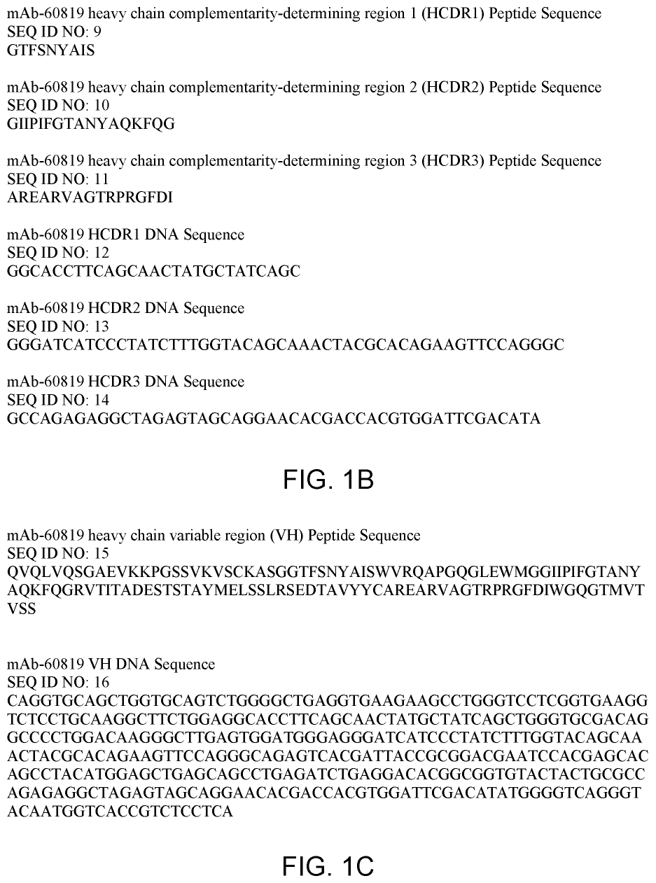

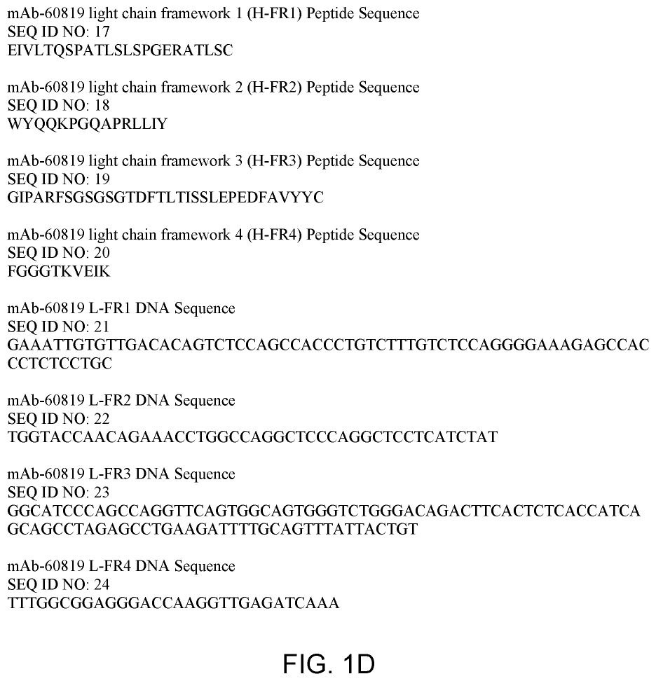

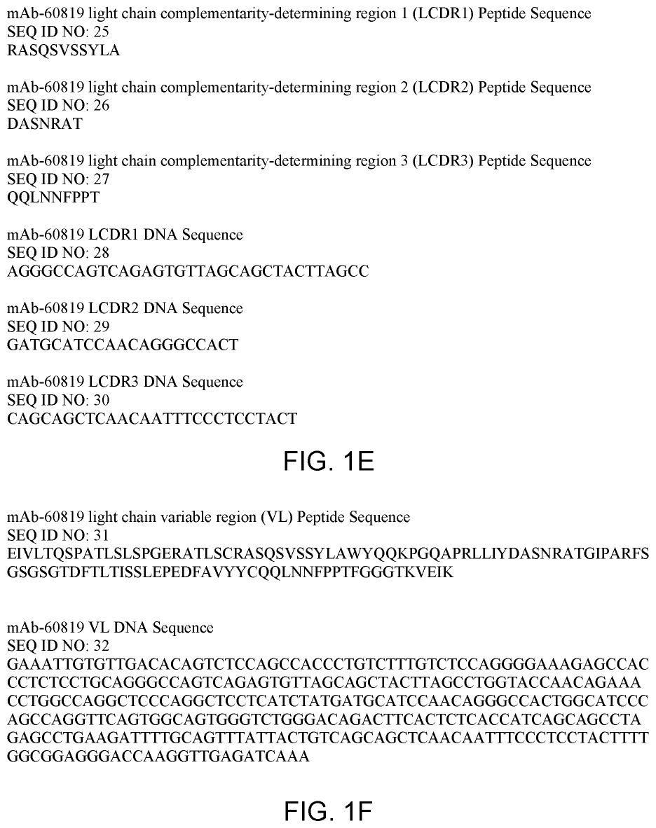

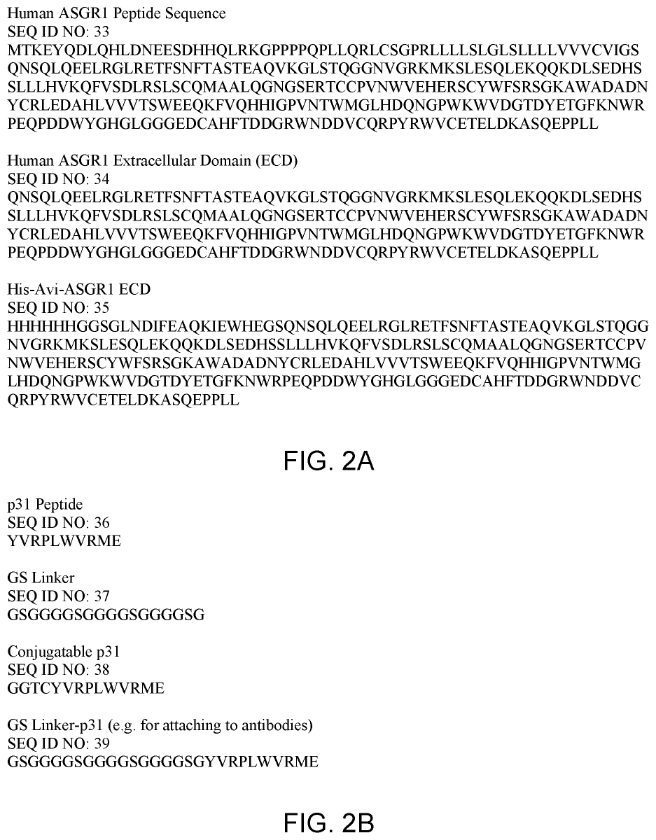

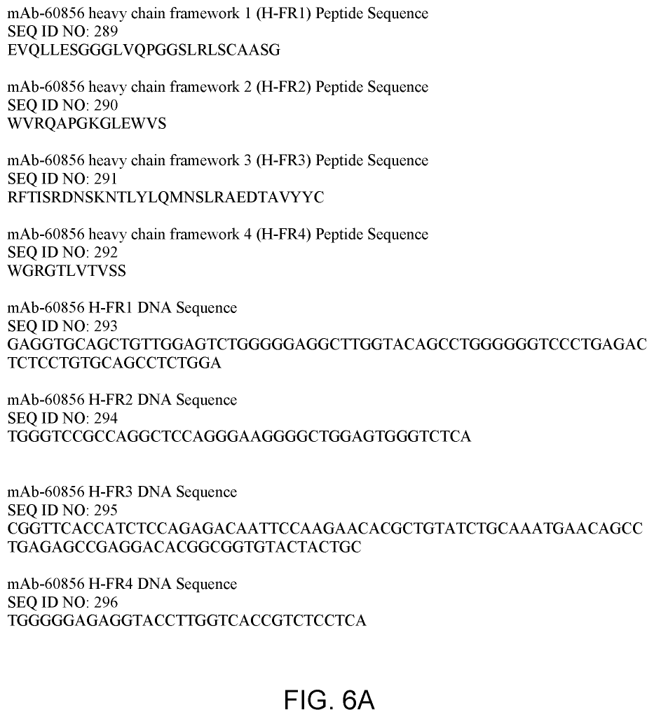

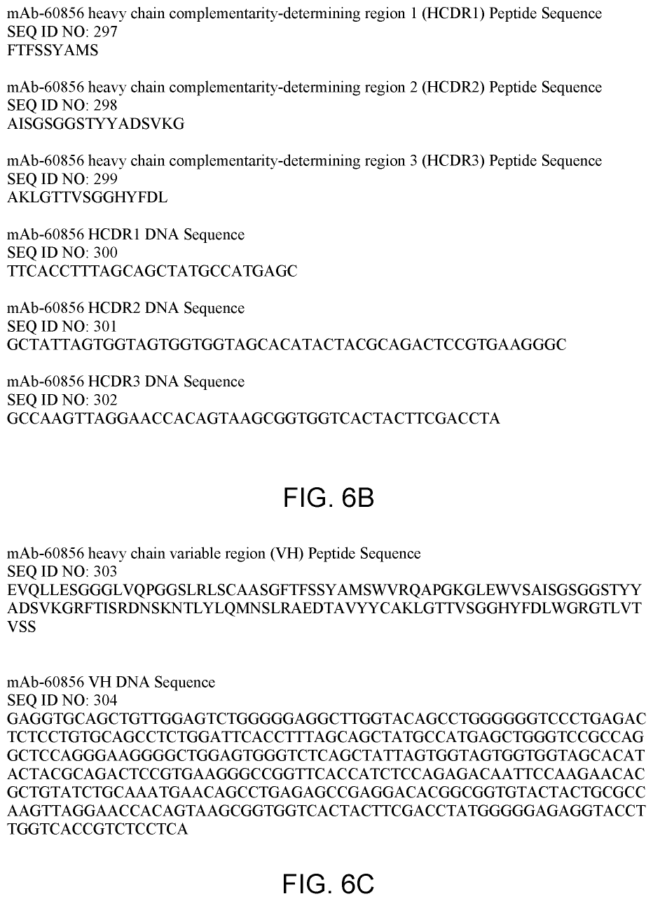

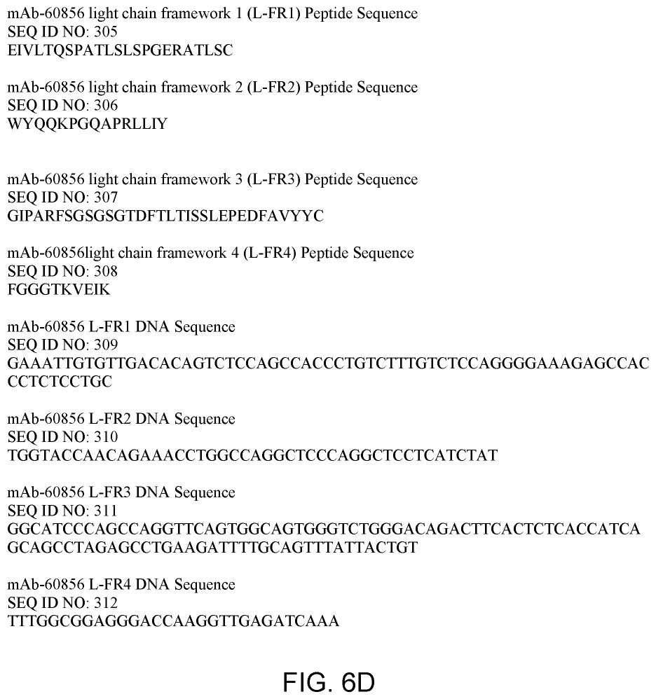

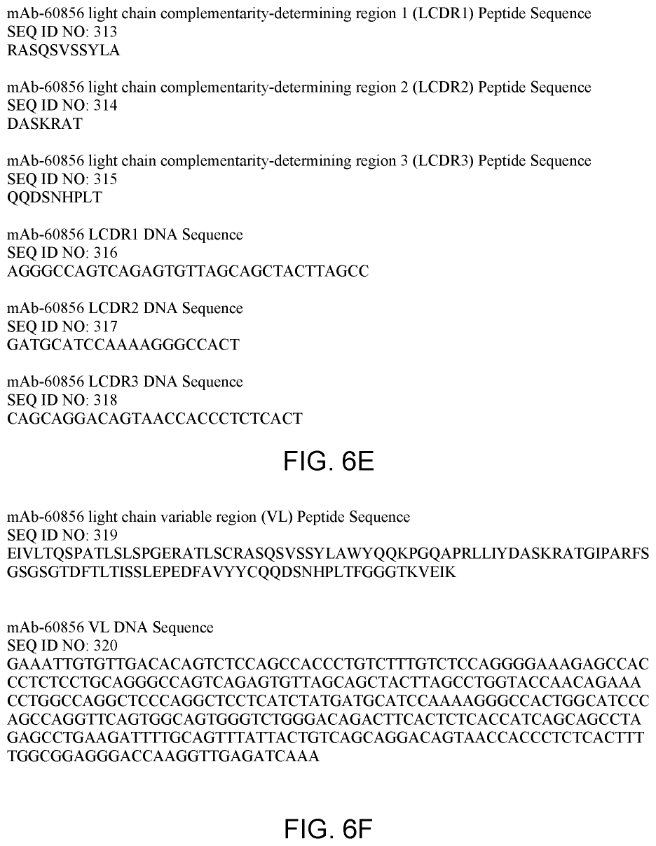

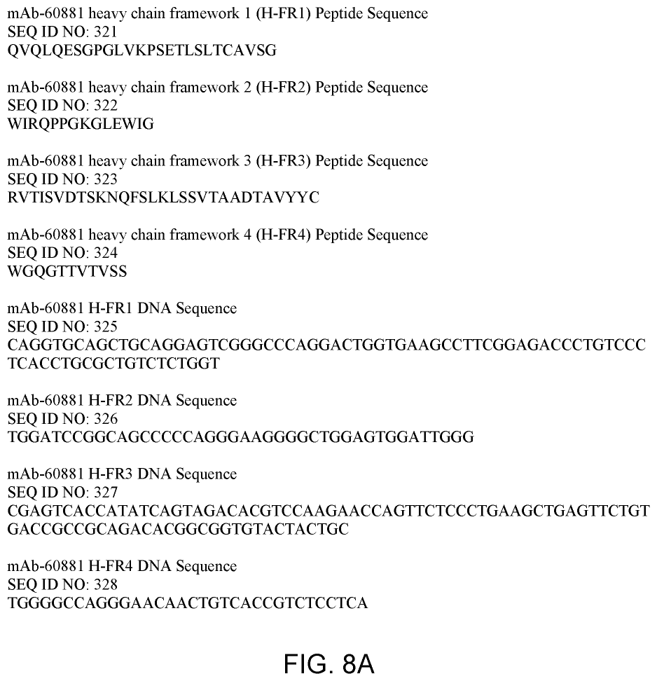

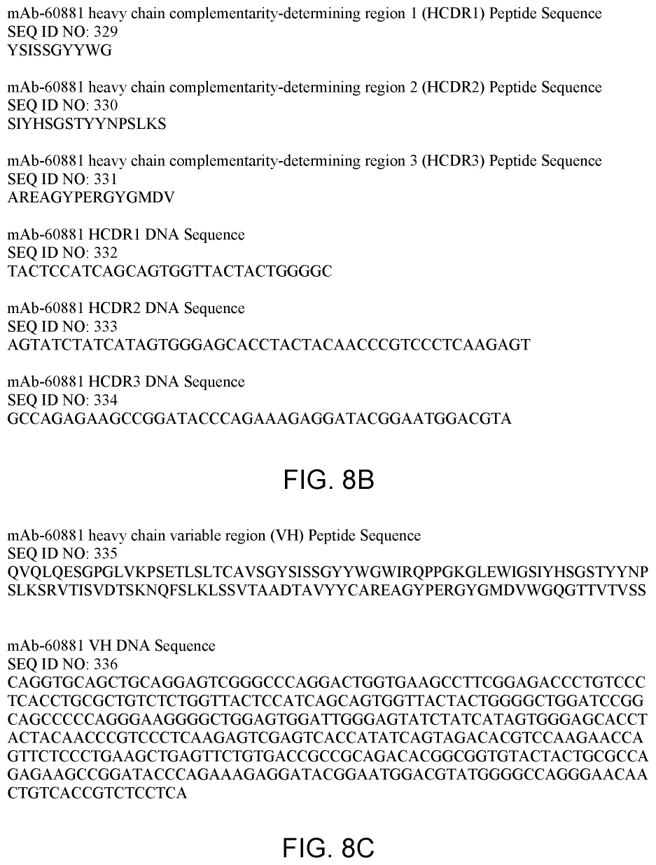

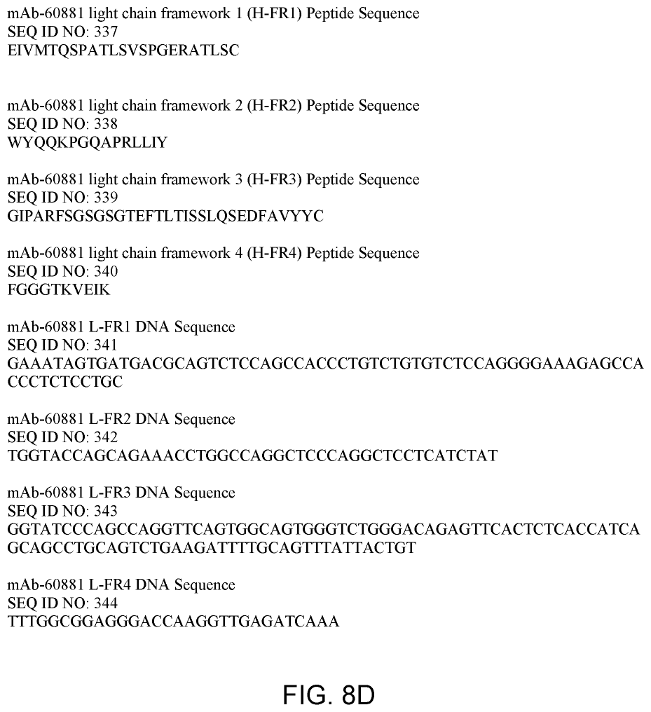

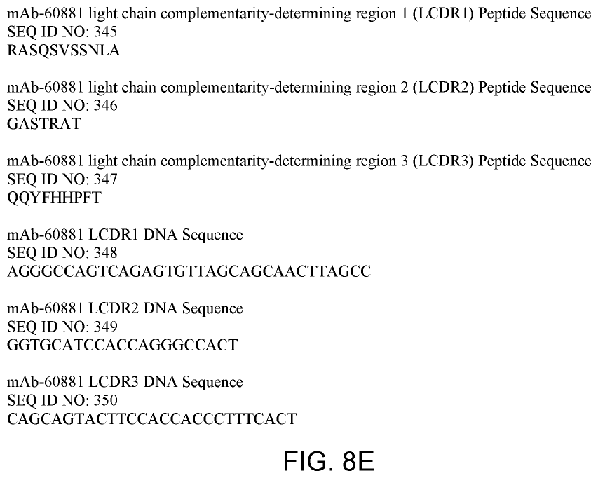

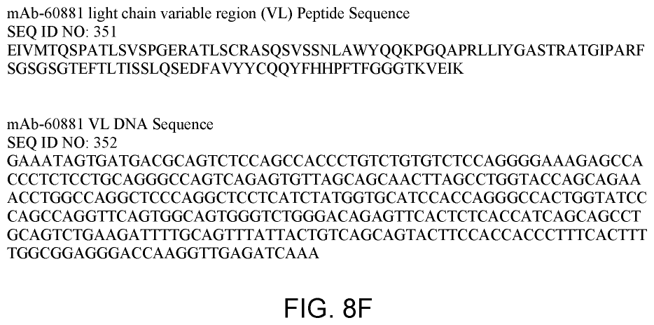

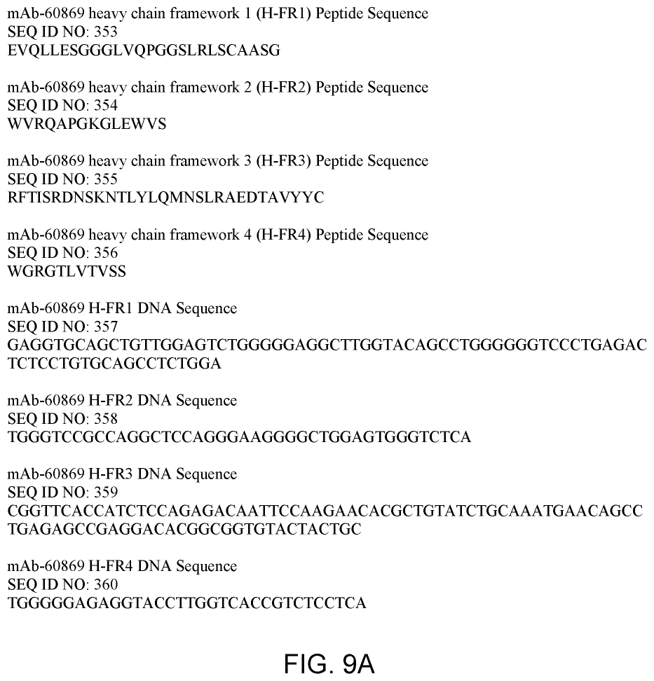

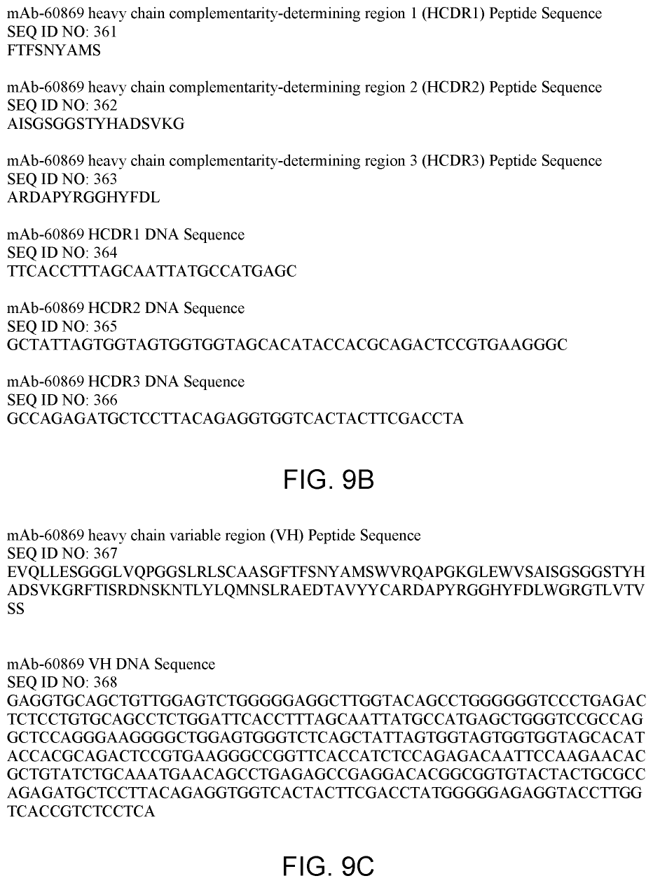

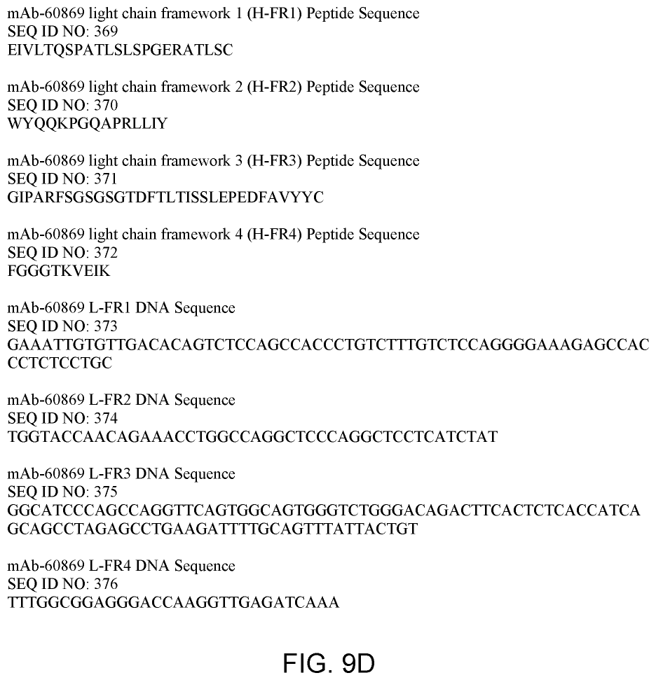

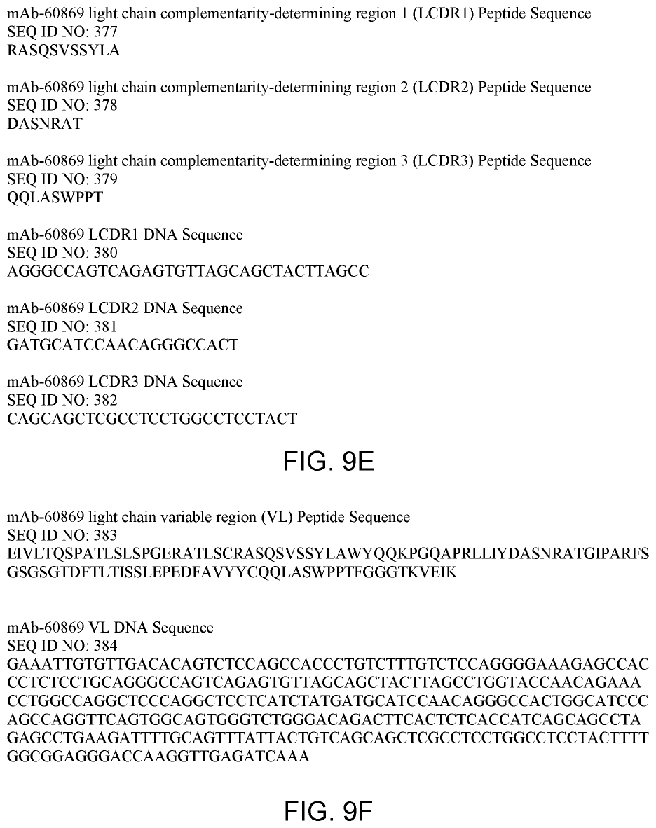

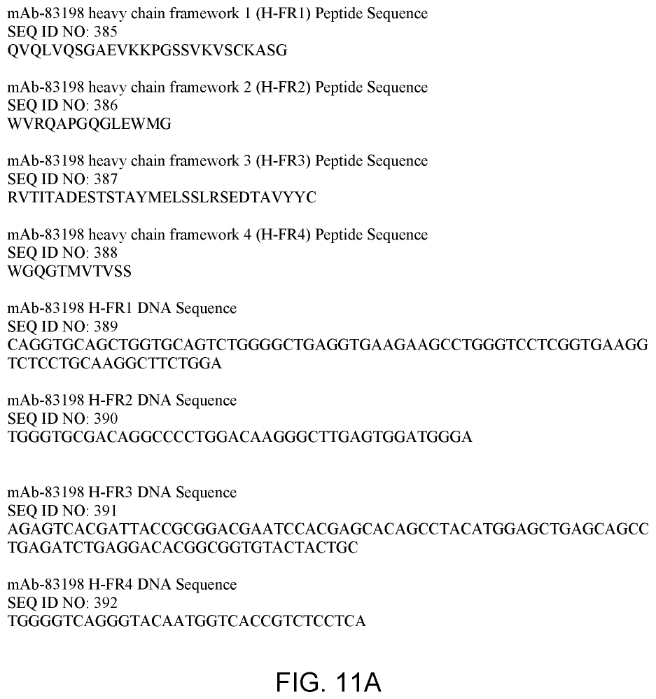

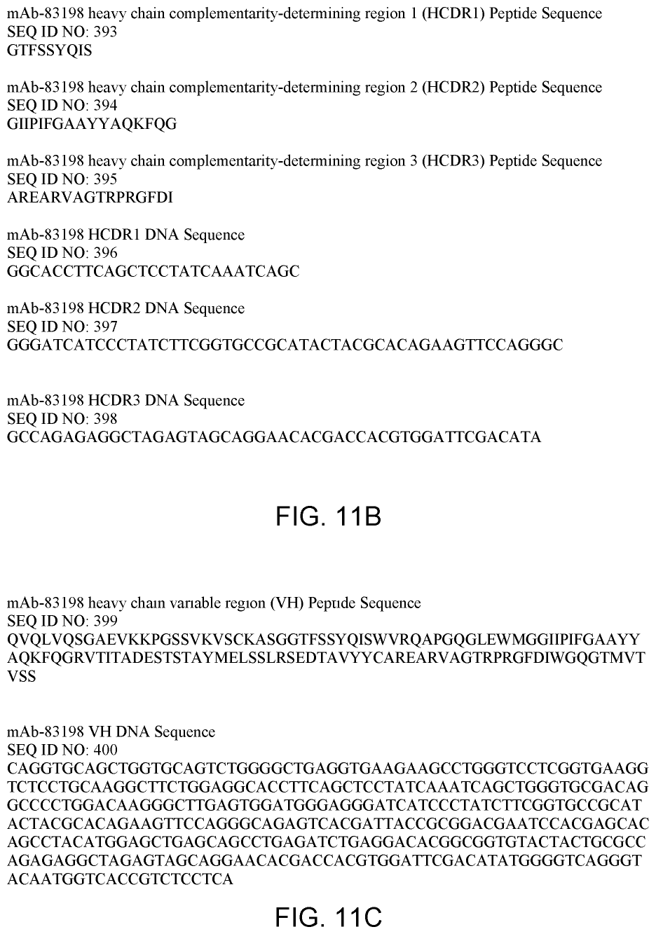

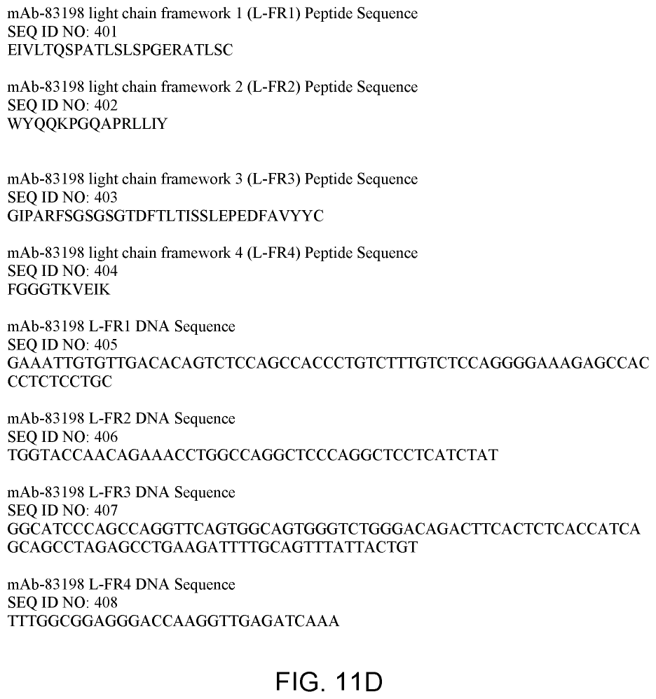

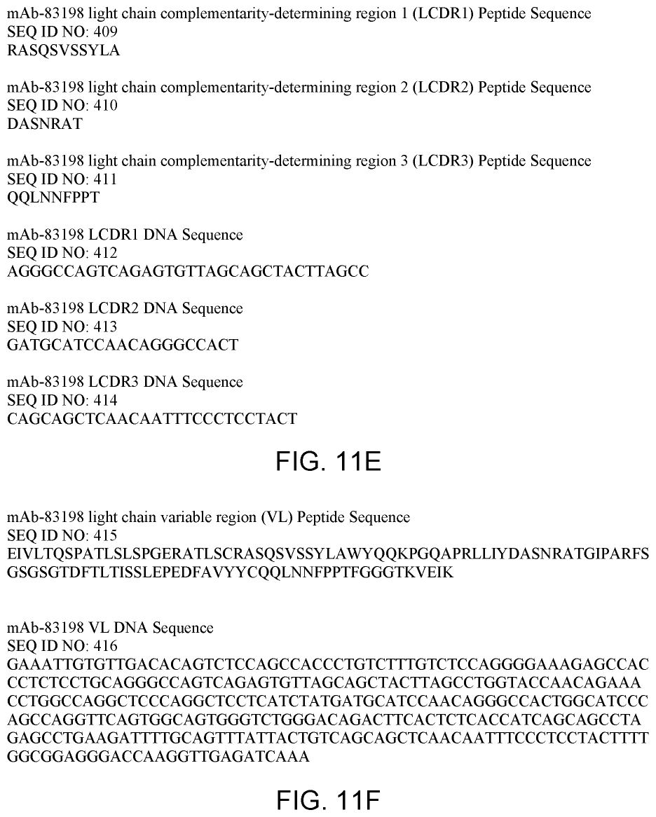

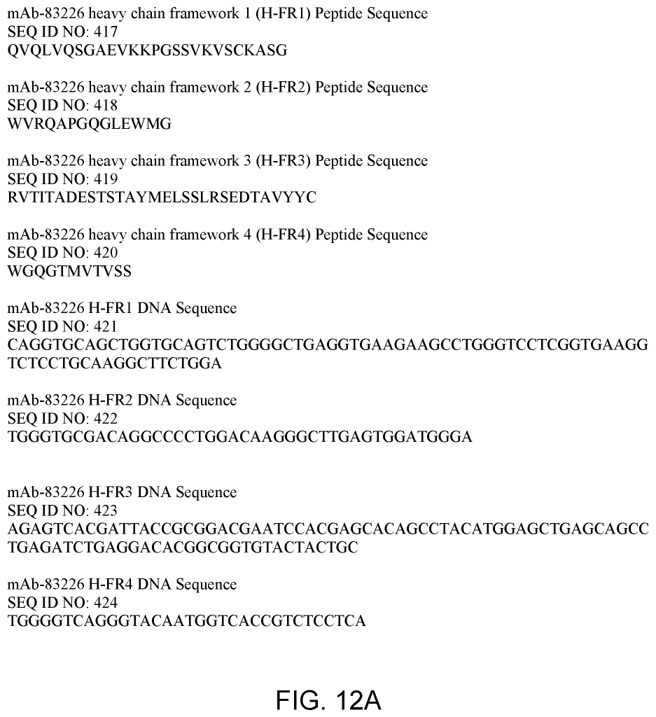

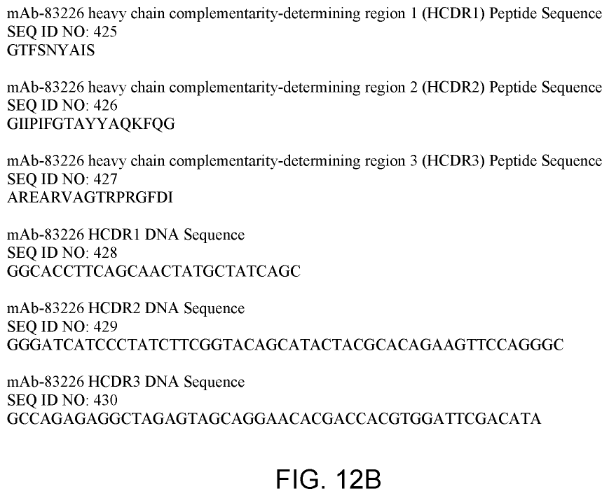

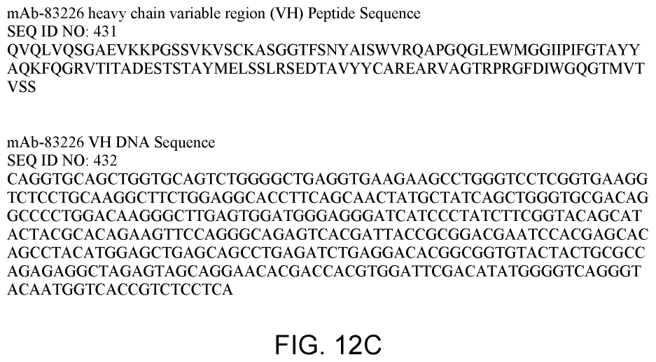

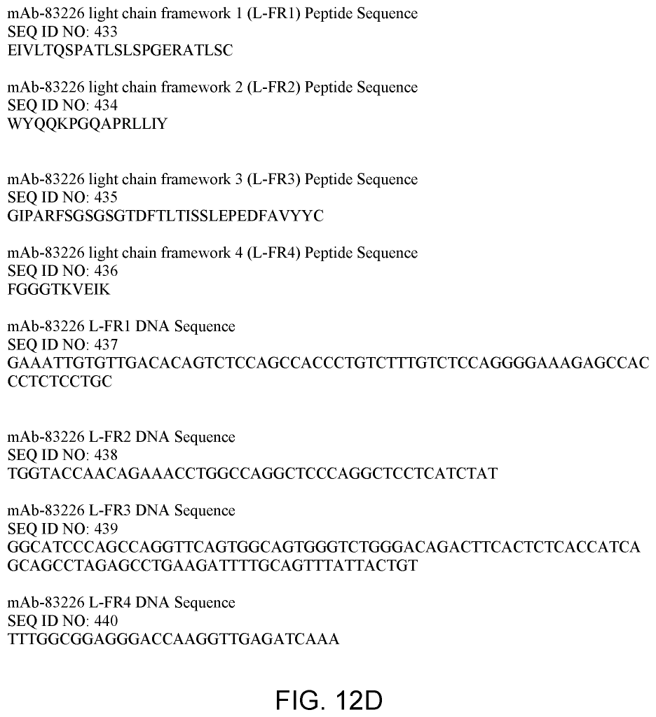

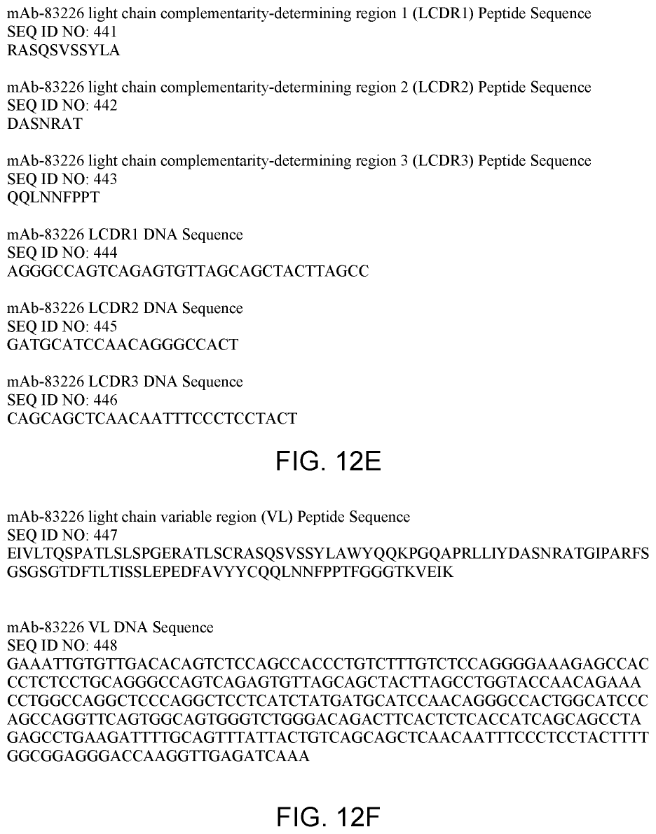

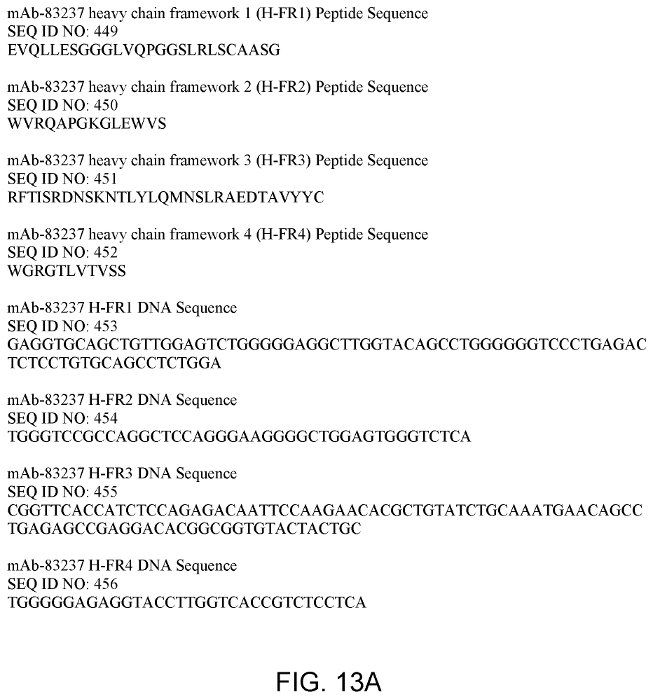

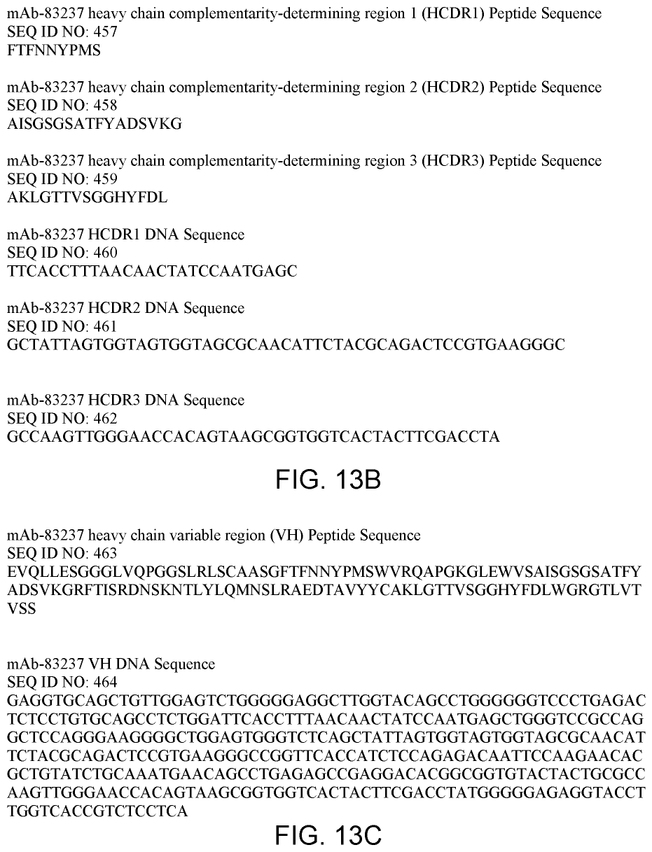

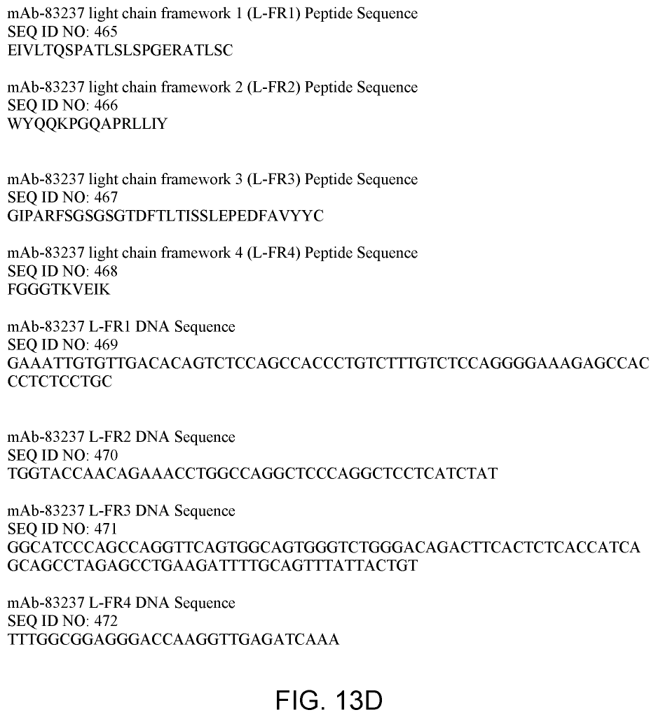

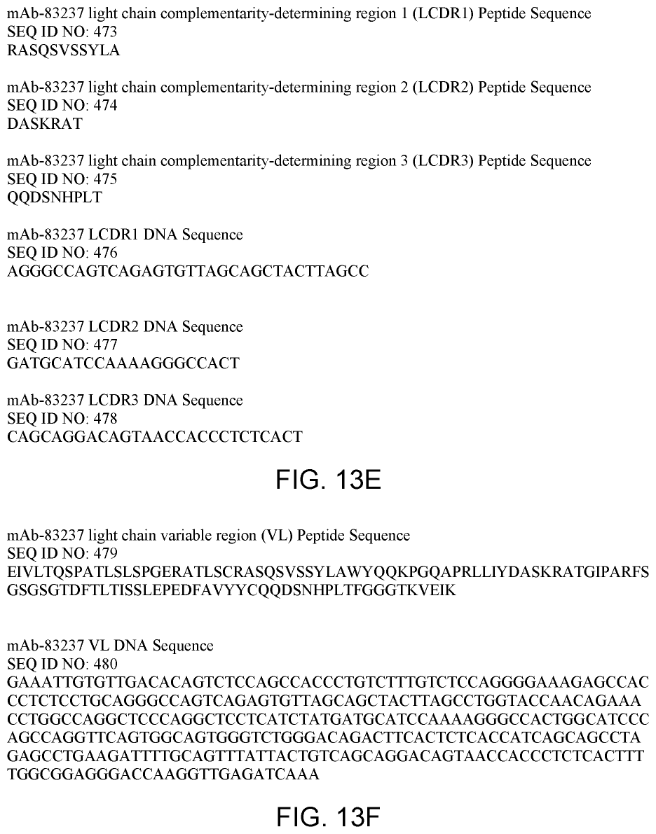

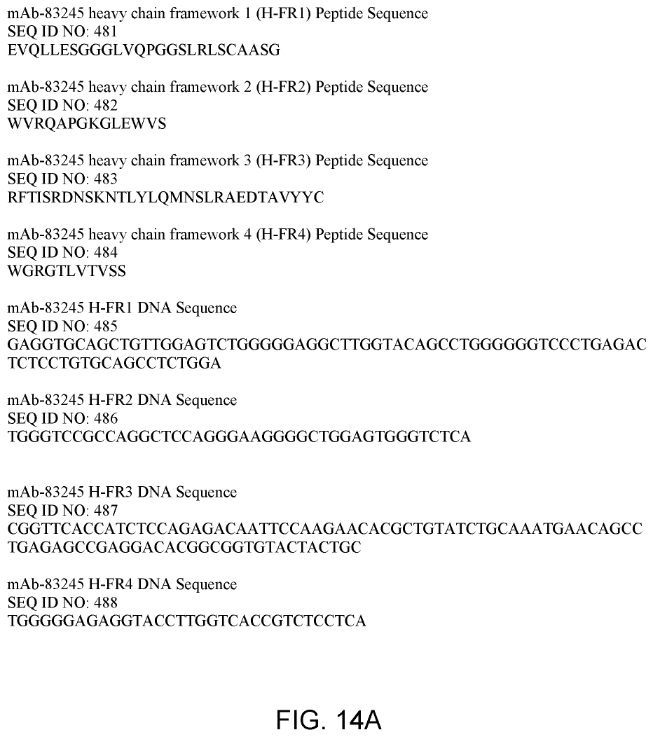

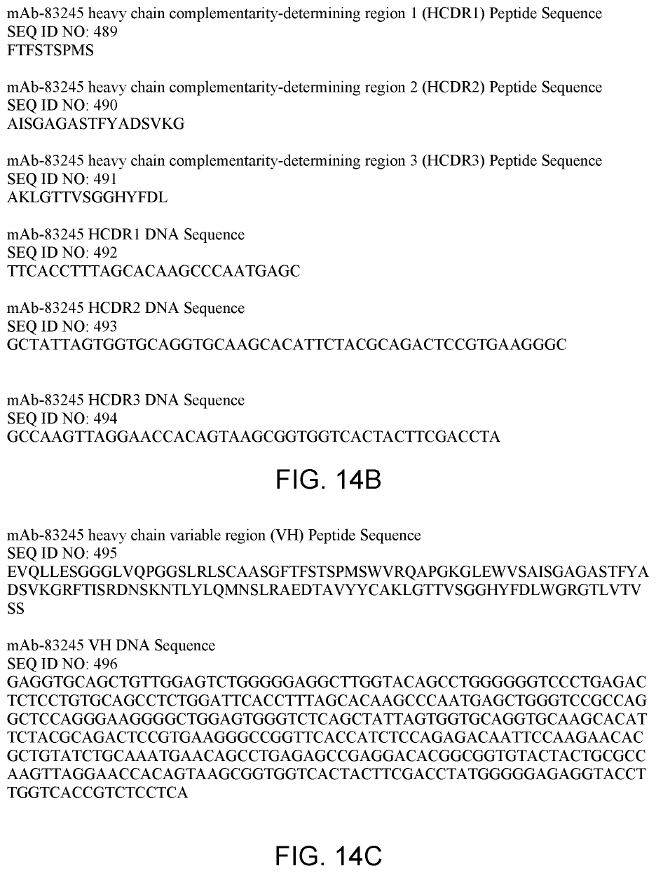

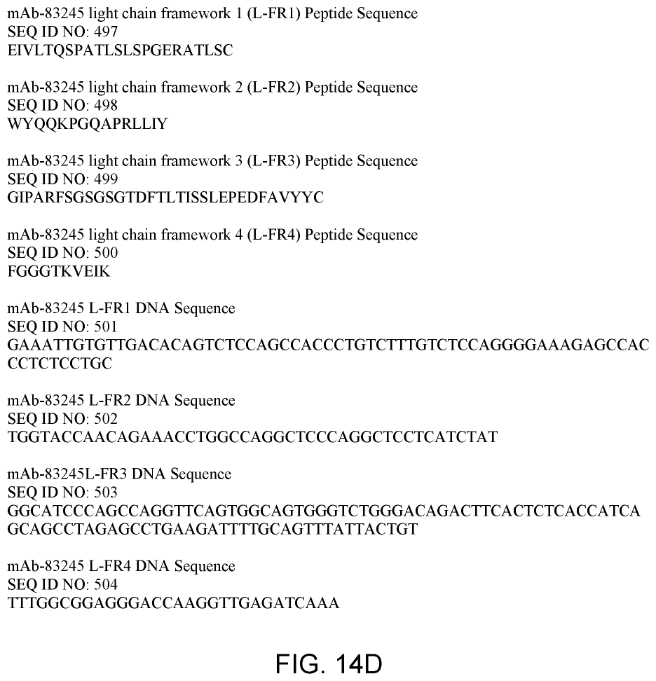

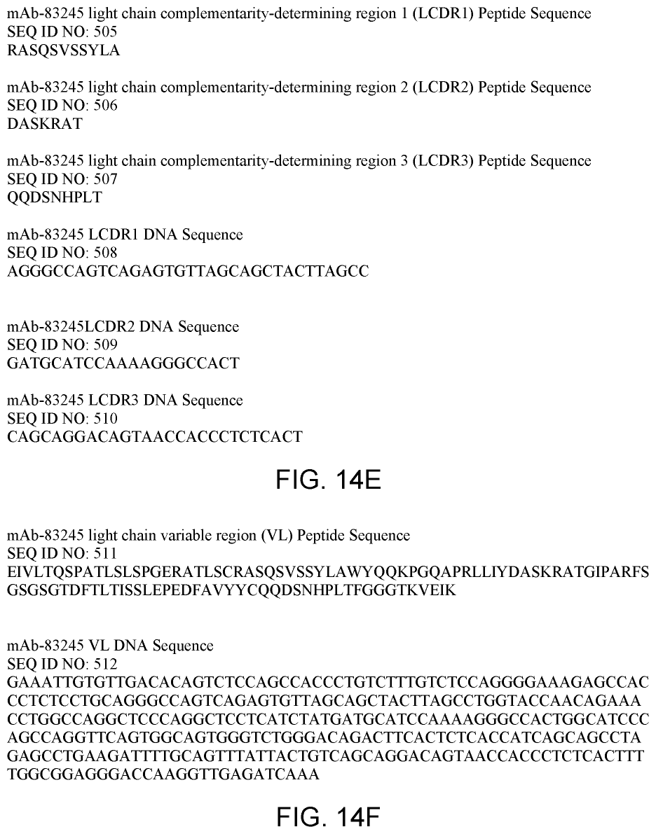

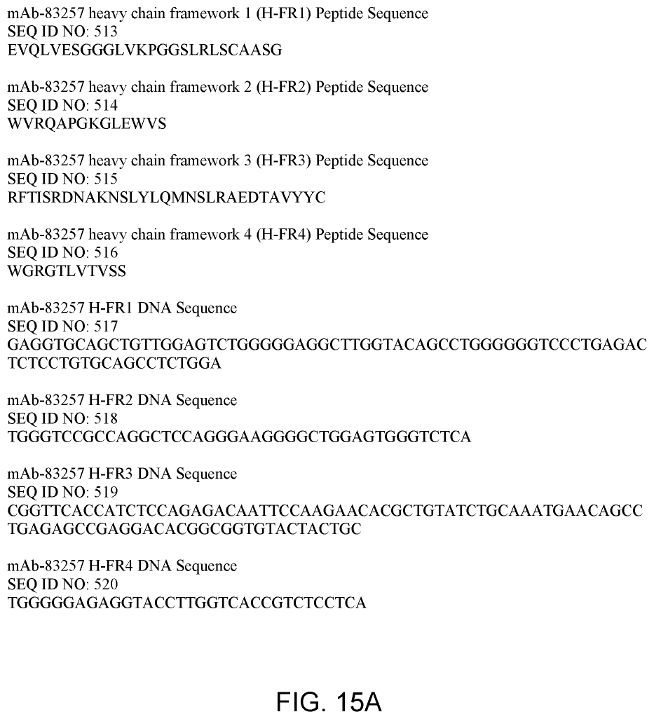

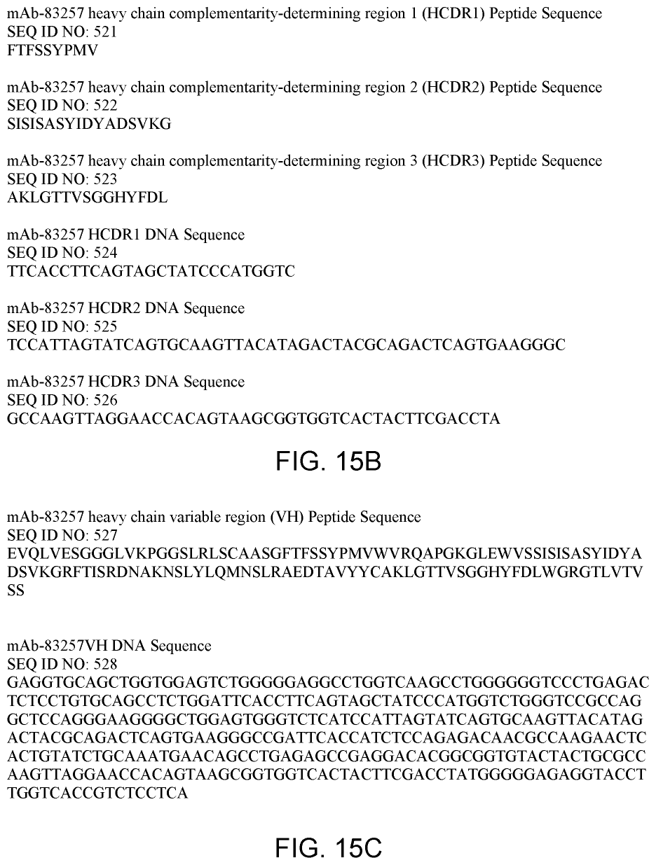

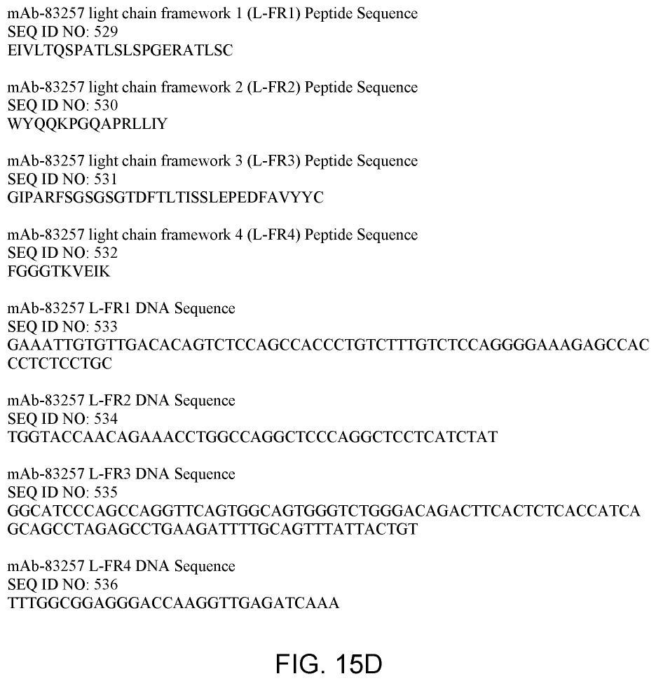

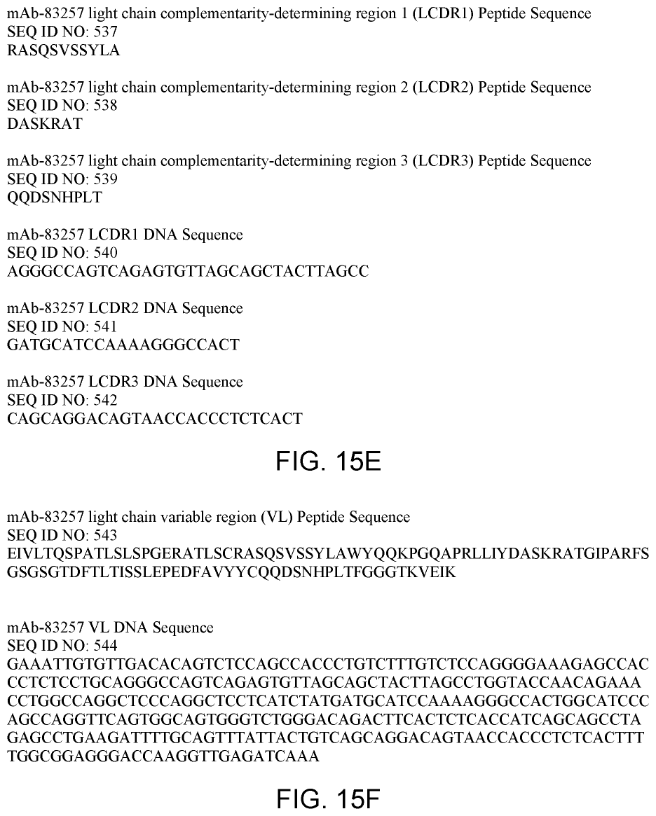

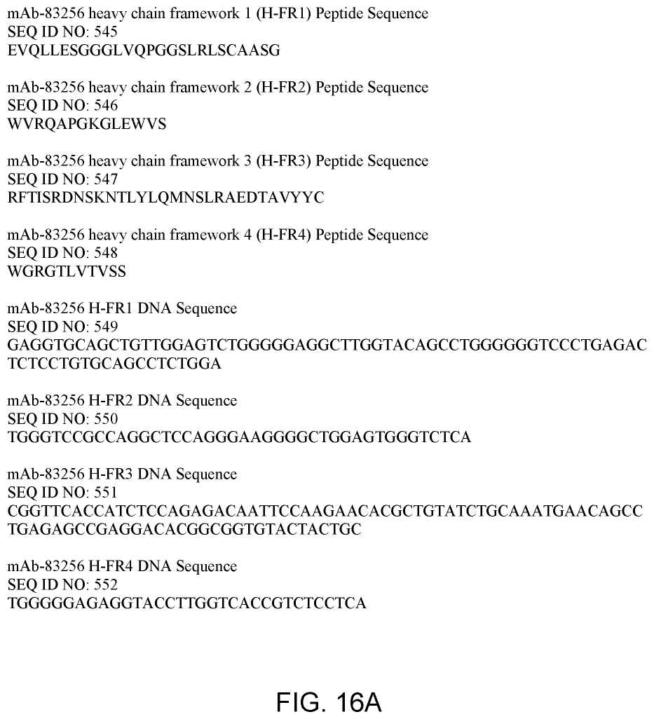

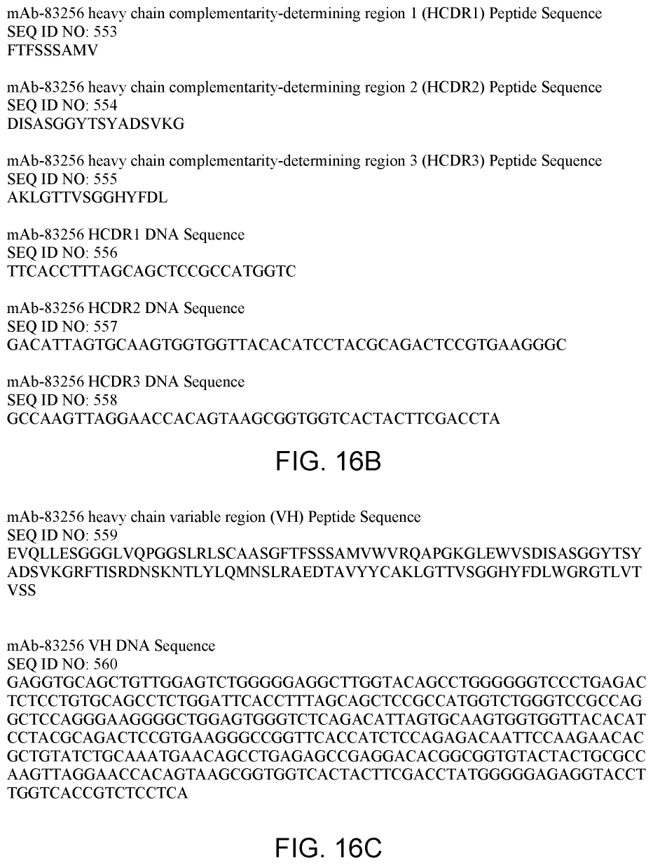

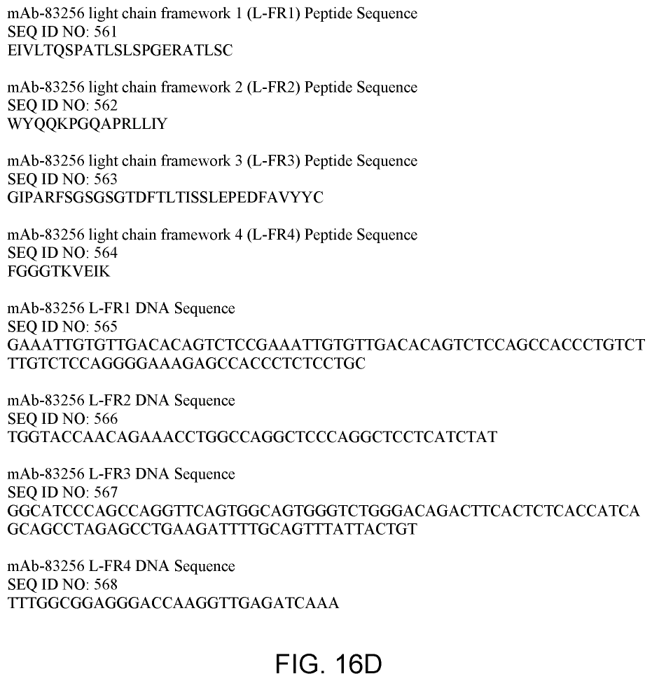

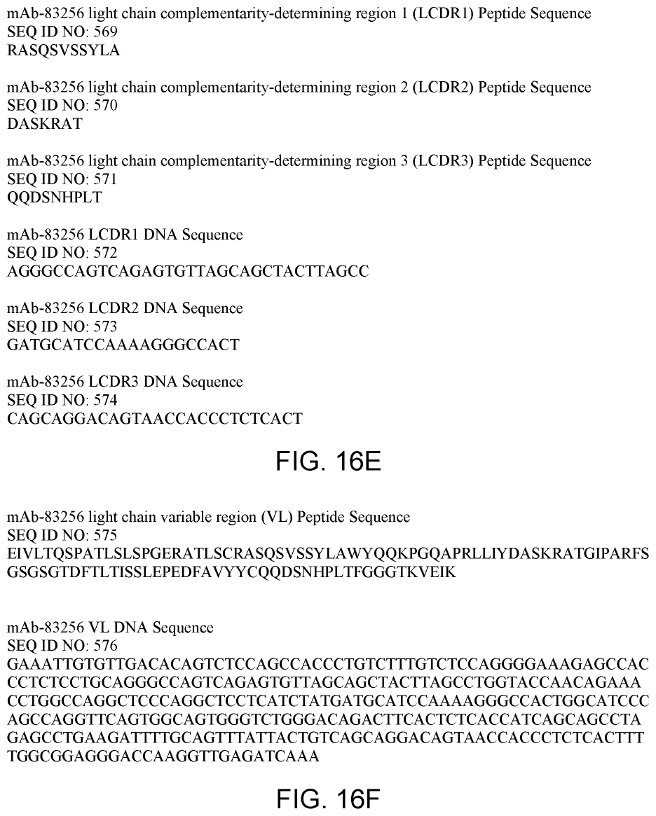

Several non-limiting examples of anti-ASGR1 antibodies were identified by this process. The antibodies comprise the sequences as detailed in Table 2 and also depicted in FIGS. 1A-1F, 6A-6F, 8A-8F, 9A-9F, 11A-11F, 12A-12F, 13A-13F, 14A-14F, 15A-15F, and 16A-16F. It is envisioned that a degree of sequence divergence is permissible without a significant effect on ASGR1 binding according to conventional experimentation. The anti-ASGR1 was antibodies are referred to as mAb-60819, mAb-60856, mAb-60881, mAb-60869, mAb-83198, mAb-83226, mAb-83237, mAb-83245, mAb-83257, mAb-83256, and are referred to elsewhere herein by their terminal three digits, e.g., “819”.

TABLE 2
Anti-ASGR1 antibody sequences

| Component | Peptide Sequences | DNA Sequences |
|---|---|---|
| Heavy chain | SEQ ID NO: 9, | SEQ ID NO: 12, |
| CDR1 (HCDR1) | 297, 329, 361, | 300, 332, 364, |
| | 393, 425, 457, | 396, 428, 460, |
| | 489, 521, 553 | 492, 524, 556 |
| Heavy chain | SEQ ID NO: 10, | SEQ ID NO: 13, |
| CDR2 (HCDR2) | 298, 330, 362, | 301, 333, 365, |
| | 394, 426, 458, | 397, 429, 461, |
| | 490, 522, 554 | 493, 525, 557 |
| Heavy chain | SEQ ID NO: 11, | SEQ ID NO: 14, |
| CDR3 (HCDR3) | 299, 331, 363, | 302, 334, 366, |
| | 395, 427, 459, | 398, 430, 462, |
| | 491, 523, 555 | 494, 526, 558 |
| Heavy chain | SEQ ID NO: 15, | SEQ ID NO: 16, |
| variable | 303, 335, 367, | 304, 336, 368, |
| region (VH) | 399, 431, 463, | 400, 432, 464, |
| | 495, 527, 559 | 496, 528, 560 |
| Light chain | SEQ ID NO: 25, | SEQ ID NO: 28, |
| CDR1 (HCDR1) | 313, 345, 377, | 316, 348, 380, |
| | 409, 441, 473, | 412, 444, 476, |
| | 505, 537, 569 | 508, 540, 572 |
| Light chain | SEQ ID NO: 26, | SEQ ID NO: 29, |
| CDR2 (HCDR2) | 314, 346, 378, | 317, 349, 381, |
| | 410, 442, 474, | 413, 445, 477, |
| | 506, 538, 570 | 509, 541, 573 |
| Light chain | SEQ ID NO: 27, | SEQ ID NO: 30, |
| CDR3 (HCDR3) | 315, 347, 379, | 318, 350, 382, |
| | 411, 443, 475, | 414, 446, 478, |
| | 507, 539, 571 | 510, 542, 574 |
| Light chain | SEQ ID NO: 31, | SEQ ID NO: 32, |
| variable | 319, 351, 383, | 320, 352, 384, |
| region (VH) | 415, 447, 479, | 416, 448, 480, |
| | 511, 543, 575 | 512, 544, 576 |

mAb-60819 was biochemically characterized and was found to have an apparent KD (KDₐₚₚ) of 9.29×10⁻⁹ M. mAb-60856 was biochemically characterized and was found to have an apparent KD (KDₐₚₚ) of 9.29×10⁻⁹ M. mAb-60881 was biochemically characterized and was found to have an apparent KD (KDₐₚₚ) of about 3×10⁻⁹ M. mAb-60869 was biochemically characterized and was found to have an apparent KD (KDₐₚₚ) of 3.22×10⁻⁹ M.ASGR1 is dependent on calcium (Ca²⁺ ions) for carbohydrate binding. The protein has an active site where the carbohydrate binds with an active site calcium, as well as calcium ions at a proximal site and distal site, and all of these calcium ions stabilize the protein structure. Accordingly, the binding of the anti-ASGR1 antibody was assessed for pH and Ca²⁺ dependency by surface plasmon resonance with a Biacore system, bio-layer interferometry with an Octet system, and with cell based assays. mAb-60819 was found to be pH and Ca²⁺ dependent, such that substantially reduced binding is detected in the absence of calcium. mAb-60856, mAb-60881 and mAb-60869 were found to be pH and Ca²⁺ independent, such that binding of these antibodies to an ASGR1 target was not affected by the absence or removal of calcium ions.

### Example 3: Induction of Immune Tolerance in Type 1 Diabetes Model

Novel liver-targeting immune tolerance platforms in which antigen-conjugated glycopolymers are specifically targeted to the liver to induce antigen-specific immune tolerance have been previously described in alternative forms and different capacities in U.S. Pat. No. 10,821,157, US Publication 2020/0101146, and PCT Publication WO 2021/053589, each of which is hereby expressly incorporated by reference in its entirety. This liver-targeting technology induces immune tolerance by deleting antigen-specific T cells, reducing antigen-specific inflammatory cytokine production, and enhancing regulatory T cells (Tregs). A novel antigen delivery platform using monoclonal antibodies (mAbs) specific to ASGR1 (αASGR1), which is expressed by liver parenchymal cells including hepatocytes, was envisioned and developed. It was examined whether antigen targeted to the liver using αASGR1 antibodies induced immune tolerance in the BDC2.5 mouse model of type 1 diabetes (T1D). In this model, targeting antigen to the liver using a non-limiting example of an αASGR1 antibody (mAb1) delayed diabetes onset, which in this model is indicative of the induction of antigen-specific immune tolerance.

The BDC2.5 TCR transgenic mouse model of T1D (NOD.Cg-Tg (TcraBDC2.5, TcrbBDC2.5) 1Doi/DoiJ [Jackson Laboratories]) was used. BDC2.5 TCR transgenic mice harbor diabetogenic CD4+ T cells specific for a region of chromogranin A (ChgA) that is post-translationally associated with the c-peptide fragment of proinsulin. BDC2.5 CD4+ T cells also recognize the major histocompatibility class II (MHC-II) I-A^{g7}-restricted polypeptide p31, which is one of several non-native mimotope peptides recognized by BCD2.5 T cells. I-A^{g7} is the mouse major histocompatibility complex that predisposes non-obese diabetic mice to develop autoimmune diabetes. When adoptively transferred into NOD.SCID mice, BDC2.5 transgenic T cells induce T1D pathology marked by insulitis and hyperglycemia. As described herein, it was assessed whether p31 delivered to the liver using αASGR1 mAbs induces antigen-specific tolerance to prevent and/or delay disease in this very aggressive model of T1D.

The BDC2.5 mouse model of T1D was used to assess the efficacy of recombinantly-expressed αASGR1-p31 fusion as a tolerogen. In this model, activated BDC2.5 TCR transgenic splenocytes were adoptively transferred into non-obese diabetic (NOD) mice carrying the severe combined immunodeficiency (SCID) mutation. Diabetes onset is observed 10 to 14 days post-adoptive transfer in control group animals that receive administrations of vehicle (saline).

NOD.SCID (NOD.Cg-Prkdc<scid>/J [Jackson Laboratories]): these immunodeficient mice do not develop mature T and B lymphocytes due to a mutation in the protein kinase PRKDC, required for the VDJ recombination of the antigen-receptor genes in germline cells. NOD.SCID mice do not spontaneously develop diabetes unless they receive an adoptive transfer of diabetogenic T cells.

The main goal was to test whether the delivery of the p31 peptide to the liver using αASGR1 mAb would confer protection from diabetes onset. On day 0, 3×10⁵ BDC2.5 splenocytes, pre-activated for 4 days with 0.5 μM p31 in vitro, were adoptively transferred intravenously (i.v.) into NOD.SCID mice. On days 0 and 4 post-cell transfer, NOD.SCID recipient mice received i.v. administrations of αASGR1-p31, free p31 peptide, or saline. Mice were monitored 2-3 times weekly for blood glucose levels. Diabetes onset was defined as two consecutive blood glucose measurements ≥250 mg/dL.

Antigen Design and Tolerogen Preparation: Tolerogens used in this study were designed to include the p31 mimotope sequence (YVRPLWVRME (SEQ ID NO: 36)) recognized by BDC2.5 CD4⁺ T cells. The αASGR1 antibody-p31 tolerogen (mAb1-p31) included a glycine-serine linker (for example, SEQ ID NO: 37) between the C-terminus of the heavy chain and p31 antigen (for example, SEQ ID NO: 39).

mAb1-p31: p31 was recombinantly expressed and fused downstream to the terminus of an example αASGR1 mAb (mAb1) heavy chain, with a short glycine-serine (GS) linker. Other example antibodies specific for ASGR1 and/or ASGR2 can be found in U.S. Pat. No. 9,771,427 and 10,358,497, each of which is hereby expressly incorporated by reference in its entirety. An example of a GS linker-p31 peptide sequence that can be fused to an αASGR1 antibody heavy chain (or other permissible regions of the antibody) is depicted in SEQ ID NO: 39. The DNA sequence corresponding to the GS linker and p31 mimotope was synthesized and cloned into a modified PCDNA3.4 vector containing DNA corresponding to the αASGR1 antibody. αASGR1-p31 was produced from a 5-day 480 mL-scale transient transfection in Expi293 cells using the manufacturer's protocol. Cell supernatants were harvested by centrifugation and filtered through a 0.2 μm aPES membrane filter unit to remove cells and debris before antibody purification.

Animals and in vivo Procedures: These studies followed animal guidelines approved by an Institutional Animal Care and Use Committee. Upon arrival, recipient NOD.SCID mice were taken randomly from their shipping container and allocated into cages by husbandry staff.

Isolation and Activation of Splenocytes for Adoptive Transfer: BDC2.5 spleens were collected and processed into cell suspension prior to activation with p31 peptide. In brief, spleen was collected in 5 ml of Iscove's Modified Dulbecco's Medium (IMDM) and disrupted by gently grinding the spleen with a syringe plunger through a 70 μm cell strainer. The cell strainer was then washed with 5 mL of IMDM, and the cell suspension was centrifuged for 5 minutes at 500×g at 4° C. The supernatant was aspirated, and the pellet was disrupted and resuspended in 1 mL ammonium-chloride-potassium (ACK) red blood cell lysis buffer and incubated for 2 minutes at room temperature. Cells were washed twice with IMDM, transferred through a 30 μm filter into a new 15 ml conical tube, and counted. Cells were centrifuged again and resuspended in IMDM culture media. Splenocytes were then cultured for 4 days with 0.5 UM of p31 peptide at a cell density of 1×10⁶ cells/mL at 37° C. with 5% CO₂. Activated BDC2.5 splenocytes were washed in IMDM culture media, resuspended at a density of 3×10⁶ cells/ml and mice were i.v. injected with 0.1 mL of the cell suspension (adoptive transfer).

Blood Glucose Measurements: Blood was collected from mice by nicking the tip of the tail with an insulin syringe. Blood glucose levels were determined using a handheld glucometer (Lifescan, Inc.) and glucose test strips (GenUltimate). The day of diabetes onset is noted as the first of two consecutive blood glucose measurements ≥250 mg/dL. Diabetic mice were euthanized.

Procedural outline for the BDC2.5 model: Twenty-one (21) NOD.SCID female mice were used as recipients, and one (1) BDC2.5 female mouse was used as a splenocyte donor. Recipient mice were distributed into three (3) experimental groups (Table 3). Mice were monitored 2-3 times weekly using a handheld glucometer. The following procedures were performed during the in vivo portion of the study:

Day −4: Splenocytes were isolated from BDC2.5 mice and cultured for 4 days with 0.5 UM of p31 peptide at a cell density of 1×10⁶ cells/ml at 37° C. with 5% CO₂.

Day 0: Recipient NOD.SCID mice were assigned to 4 groups (Table 3). Cultured splenocytes were harvested, washed, and prepared into single-cell suspensions. 3×10⁵ cells/mouse were adoptively transferred i.v. into all recipients.

Days 0 and 4: Mice were injected i.v. with mAb1-p31, p31, or saline (Table 3). Doses were calculated based on the average body weight (21.3 g) of all recipient mice in the study. Baseline body weight and blood glucose were measured 2 days before the beginning of the study.

Day 4-105: Mice were monitored for T1D by twice-weekly blood glucose measurements. Mice were considered diabetic after two consecutive blood glucose measurements ≥250 mg/dL.

TABLE 3
Experimental groups for the BDC2.5 model

| | | | | Test | |
|---|---|---|---|---|---|
| | | BDC2.5 | | Article | Dose |
| | Number of | Splenocytes | Test | Dose | Volume |
| Group | mice | (3 × 10⁵ cells) | Article | (pmol/g) | (mL) |
| 1 | 7 | Day 0 | mAb1-p31 | 50 | 0.1 |
| 2 | 7 | Day 0 | p31 | 50 | 0.1 |
| 3 | 7 | Day 0 | Saline | n/a | 0.1 |

### Results:

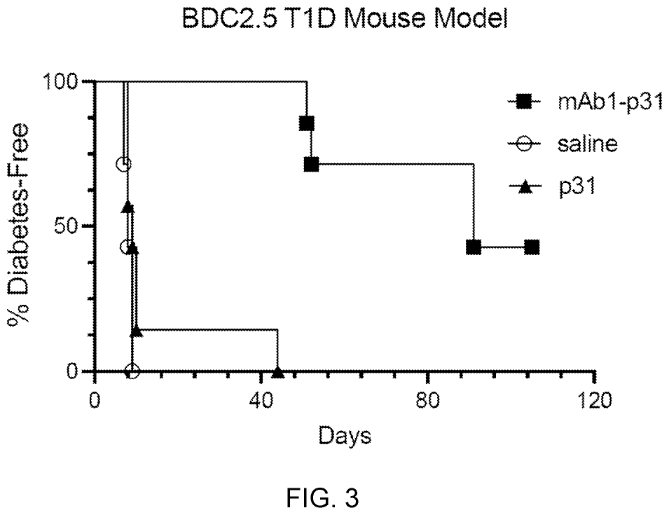

Liver-targeted p31 Antigens Induce Immune Tolerance and Prolong Diabetes-Free Survival in a BDC2.5 model of T1D: Animals treated with mAb1-p31 showed prolonged diabetes-free survival (median survival time [MST]=91 days) when compared to p31 and saline treated control groups (MST=9 and 8 days respectively) (p=0.0001, FIG. 3, Table 4; Means were compared to the p31 treated control group using the Mantel-Cox log-rank test). These data demonstrate that αASGR1 mAb1-p31 protect mice from T1D by inducing p31-specific immune tolerance even when the diabetogenic T cells are highly pre-activated prior to adoptive transfer.

TABLE 4
Survival statistics for the BDC2.5 model of T1D

| | | Median Diabetes- | |
|---|---|---|---|
| | | Free Survival | |
| Group | Tolerogen | Time (days) | p-value |
| 1 | mAb1-p31 | 91 | 0.0001 |
| 2 | p31 | 9 | n/a |
| 3 | Saline | 8 | 0.093 |

### Example 4: Immune Tolerance in the BDC2.5 Mouse T1D Model Using a Non-Limiting Embodiment of an Anti-ASGR1 Antibody

Anti-ASGR1 antibodies mAb-60819, mAb-60856, mAb-60869 as described herein (e.g. VH having the sequences of SEQ ID NOs: 15, 303, 367 and VL having the sequence of SEQ ID NOs: 31, 319, 383 respectively) are used to prepare a p31 tolerogen for testing in the BDC2.5 T1D mouse model. The p31 peptide is fused with a glycine-serine linker to the C-terminal end of the antibody heavy chain.

Optionally, effector function of the Fc region of the antibody may be silenced such that it does not bind to FcRn or other FcRγ receptors. The Fc portion of the antibody is not needed for the desired immune tolerance effect.

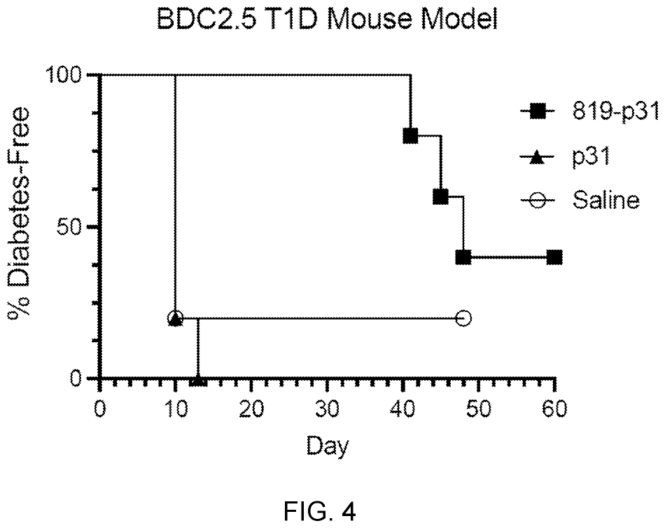

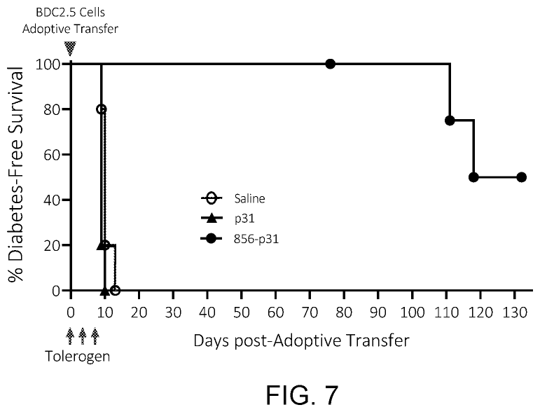

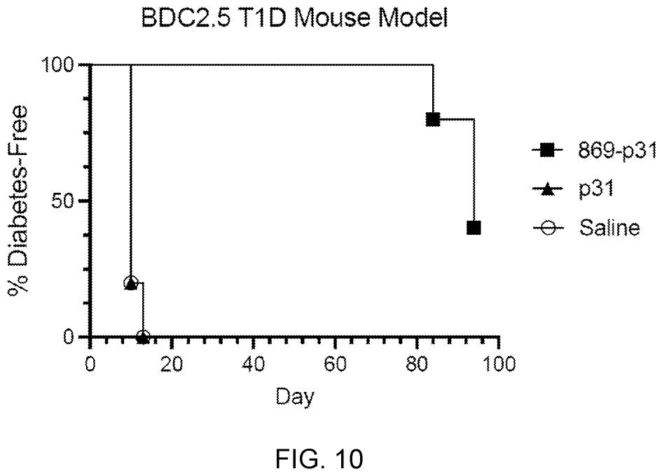

The anti-ASGR1 antibody tolerogens were administered to the NOD.SCID T1D mouse model adoptively transferred with BCD2.5 splenocytes according to the methods described in Example 2. As depicted in FIGS. 4, 7 and 10, mAb-60819, mAb-60856 and mAb-60869 significantly delayed the incidence of T1D in the NOD.SCID model compared to unconjugated p31 peptide or saline control.

### Example 5: Induction of Immune Tolerance in the EAE Mouse Model of Multiple Sclerosis

The tolerogens disclosed herein were also investigated for their impact on immune tolerance in a mouse model of human multiple sclerosis (MS). The animal model was an experimental autoimmune encephalomyelitis (EAE) adoptive transfer mouse model.

Readouts for immune tolerance include changes in the following parameters: 1. Disease severity measured by disease score (paralysis), 2. Body weight loss, 3. Inflammatory cytokine production by myelin-specific CD4+ T cells.

EAE is an established MS mouse model that has been used extensively in preclinical development of other MS therapies, in which the transfer of activated autoreactive CD4+ T cells induces MS-like disease. Given that the transferred cells being treated by the therapy are highly activated, this model closely mimics a therapeutic setting in which patients have circulating activated T cells that recognize autoantigens. The findings herein are considered generalizable to other autoimmune diseases where immunopathology is driven by autoreactive T cells, such as celiac disease.

In brief, in the EAE adoptive transfer model, donor mice are immunized to generate antigen-specific encephalitogenic T cells. The spleen cells from these mice are then activated and expanded in cell culture and transferred intraperitoneally (i.p.) into recipient mice, which induces symptomatic EAE. The antigen used for vaccination in this model is the immunodominant peptide fragment of Myelin Oligodendrocyte Glycoprotein (MOG), amino acid sequence 35-55. The analogous region of human MOG is highly homologous to mouse MOG and is also an identified T cell epitope in human MS that spans amino acids 34-56 of human MOG as in mouse EAE. As disclosed herein, other fragments of human MOG may be used in several embodiments, such as MOG 1-60, MOG 33-62, or any other MOG fragment disclosed herein or otherwise known in the art.

Efficacy in the EAE model is measured by the reduction in several clinical parameters including:

1) EAE incidence, which is the percent of mice that develop disease at any point during the study regardless of disease severity or subsequent remission.

2) The mean maximum score (MMS), which is calculated as the average of the highest clinical score reached at any point during the study for each individual subject. This is considered the primary measure of diseases severity. The theoretical range is from 0 to 5 based on extent of mobility.

3) Weight loss, which is expected to positively correlate with increasing disease scores.

4) Median day of onset of disease, which is the median day on which animals in a group exhibit a clinical score greater than 0.

5) Average end score, which is the average clinical score for a group at the end of the study. As disease scores spontaneously decrease at later times during the study, this measure is considered less relevant to overall efficacy.

Antigen design: The EAE induction model involves vaccination of mice with the immunodominant peptide domain of mouse MOG (MOG 35-55; SEQ ID NO: 79); vaccination induces encephalitogenic CD4+ T cells that are subsequently expanded ex vivo and adoptively transferred into and mediate disease in recipient mice. The tolerogen may include an/the immunodominant peptide domain of MOG fused with a linker, such as a cysteine-containing linker, for conjugation to an ASGR1-binding polypeptide, or other linkers for fusing to an antibody, such as a linker containing glycine and serine residues, as contemplated herein. The homologous immunodominant peptide domain of human MOG (MOG 35-55; SEQ ID NO: 78) may be used.

EAE was induced at day 0 in C57BL/6 recipient mice by i.p. injection of encephalitogenic T cells derived from B6.SJL donor mice immunized with MOG 35-55. Test condition mice were also treated with liver-targeted MOG 35-55 antigen.

The negative control group for treatment was vehicle (saline)-treated mice, wherein mice are expected to develop EAE.

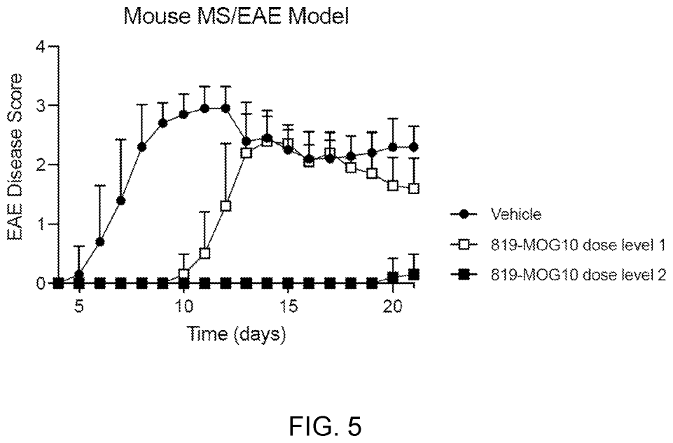

EAE model mice administered with encephalitogenic T cells were treated with MOG 33-62 (“MOG10”) conjugated to mAb-60819 (819-MOG10). 819-MOG10 was administered at either 2 pmol/g or 10 pmol/g dose magnitudes. As seen in FIG. 5, administration of 819-MOG10 at 10 pmol/g results in >90% efficacy up to 20 days after induction of EAE. This efficacy is dose dependent. These data illustrate that a myelin-derived autoantigen (MOG) delivered to the liver by way of the αASGR1 mAb-60819 induces antigen-specific immune tolerance and prevents an unwanted immune response associated with an autoimmune disease (here, as a non-limiting example, MS).

As disclosed herein, in several embodiments, targeting an antigen to which tolerance is desired, such as an antigen associated with MS, celiac disease, or type 1 diabetes, as non-limiting examples, to the liver by way of an anti-ASGR1 antibody leads to antigen-specific immune tolerance to prevent an unwanted immune response associated with a disease to which the antigen to which tolerance is desired is associated, such as MS, celiac disease, or type 1 diabetes, as non-limiting examples. As disclosed herein, in several embodiments, targeting an antigen to which tolerance is desired to the liver by way of an anti-ASGR1 antibody leads to antigen-specific immune tolerance to prevent an unwanted immune response associated with a disease to which the antigen to which tolerance is desired is associated.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” is typically interpreted as “including but not limited to,” the term “having” is typically interpreted as “having at least,” the term “includes” is typically interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases is typically construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” is typically interpreted to mean “at least one” or “one or more”); the same holds true for the use of definite articles used to introduce claim recitations. In addition, even if a specific number of an introduced claim recitation is explicitly recited, those skilled in the art will recognize that such recitation is typically interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, is typically understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

A Sequence Listing in electronic format is submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Bromilow et al. “A curated gluten protein sequence database to support development of proteomics methods for determination of gluten in gluten-free foods” J. Proteomics (2017) 163:67-75.
Delong, T., T. A. Wiles, R. L. Baker, B. Bradley, G. Barbour, R. Reisdorph, M. Armstrong, R. L. Powell, N. Reisdorph, N. Kumar, C. M. Elso, M. DeNicola, R. Bottino, A. C. Powers, D. M. Harlan, S. C. Kent, S. I. Mannering and K. Haskins (2016). Pathogenic CD4 T cells in type 1 diabetes recognize epitopes formed by peptide fusion. Science 351(6274): 711-714.
Hellings, N., M. Baree, C. Verhoeven, B. D′Hooghe M, R. Medaer, C. C. Bernard, J. Raus and P. Stinissen (2001). T-cell reactivity to multiple myelin antigens in multiple sclerosis patients and healthy controls. J Neurosci Res 63(3): 290-302.
Judkowski, V., C. Pinilla, K. Schroder, L. Tucker, N. Sarvetnick and D. B. Wilson (2001). Identification of MHC class II-restricted peptide ligands, including a glutamic acid decarboxylase 65 sequence, that stimulate diabetogenic T cells from transgenic BDC2.5 nonobese diabetic mice. J Immunol 166(2): 908-917.
Katz, J. D., B. Wang, K. Haskins, C. Benoist and D. Mathis (1993). Following a diabetogenic T cell from genesis through pathogenesis. Cell 74(6): 1089-1100.
Mendel, I., N. Kerlero de Rosbo and A. Ben-Nun (1995). A myelin oligodendrocyte glycoprotein peptide induces typical chronic experimental autoimmune encephalomyelitis in H-2b mice: fine specificity and T cell receptor V beta expression of encephalitogenic T cells. Eur. J. Immunol. 25(7): 1951-1959.
Wilson, D. S., M. Damo, S. Hirosue, M. M. Raczy, K. Brunggel, G. Diaceri, X. Quaglia-Thermes and J. A. Hubbell (2019). Synthetically glycosylated antigens induce antigen-specific tolerance and prevent the onset of diabetes. Nat Biomed Eng 3(10): 817-829.

1. An asialoglycoprotein receptor 1 (ASGR1) binding polypeptide comprising a heavy chain variable (VH) region comprising a first heavy chain complementarity determining region (HCDR1), a second heavy chain complementarity determining region (HCDR2), and a third heavy chain complementarity determining region (HCDR3) and a light chain variable region (VL) comprising a first light chain complementarity determining region (LCDR1), a second light chain complementarity determining region (LCDR2), and a third light chain complementarity determining region (LCDR3);
wherein the HCDR1 comprises the sequence of SEQ ID NO: 9;
wherein the HCDR2 comprises the sequence of SEQ ID NO: 10; and
wherein the HCDR3 comprises the sequence of SEQ ID NO: 11;
wherein the LCDR1 comprises the sequence of SEQ ID NO: 25;
wherein the LCDR2 comprises the sequence of SEQ ID NO: 26; and
wherein the LCDR3 comprises the sequence of SEQ ID NO: 27.

2. The ASGR1 binding polypeptide of claim 1, wherein the heavy chain variable region comprises a sequence having at least 95%, 96%, 97%, 98%, 99%, or 100% identity to SEQ ID NO: 15.

3. (canceled)

4. The ASGR1 binding polypeptide of claim 1, wherein the light chain variable region comprises a sequence having at least 95%, 96%, 97%, 98%, 99%, or 100% identity to SEQ ID NO: 31.

5. The ASGR1 binding polypeptide of claim 1, wherein the heavy chain variable region comprises the sequence of SEQ ID NO: 15 and the light chain variable region comprises the sequence of SEQ ID NO: 31.

6. The ASGR1 binding polypeptide of claim 1, wherein the ASGR1 binding polypeptide is an antibody, an Fab′ fragment, an F(ab′)2 fragment, a domain antibody (dAb), or an scFv.

7. The ASGR1 binding polypeptide of claim 1, wherein the ASGR1 binding polypeptide comprises an Fc domain, optionally wherein the Fc domain is silenced.

8. A polynucleotide encoding for the ASGR1 binding polypeptide of claim 1, the polynucleotide comprising one or more of the sequences of SEQ ID NO: 12-14 and comprising one or more of the sequences of SEQ ID NO: 25-27.

9. The polynucleotide of claim 8, wherein the polynucleotide comprises the sequence of SEQ ID NO: 16 and comprises the sequence of SEQ ID NO: 32.

10. (canceled)

11. (canceled)

12. (canceled)

13. A tolerogenic compound comprising an ASGR1 binding polypeptide according to claim 1, wherein the ASGR1 binding polypeptide is conjugated or fused to an antigen to which tolerance is desired.

14. The tolerogenic compound of claim 13, wherein the ASGR1 binding polypeptide and the antigen are conjugated or fused with a linker, optionally wherein the linker is a polypeptide linker or a chemical conjugation linker.

15. The tolerogenic compound of claim 14, wherein the linker is a cleavable linker.

16. The tolerogenic compound of claim 15, wherein the linker comprises glycine and/or serine, optionally wherein the linker comprises the sequence of SEQ ID NO: 37.

17. The tolerogenic compound of claim 13, wherein the antigen is conjugated or fused to the N-terminus or C-terminus of the ASGR1 binding polypeptide.

18. The tolerogenic compound of claim 13, wherein the antigen comprises a food antigen.

19. The tolerogenic compound of claim 18, wherein the food antigen is associated with celiac disease.

20. The tolerogenic compound of claim 19, wherein the antigen comprises a sequence having at least 95%, 96%, 97%, 98%, 99%, or 100% sequence identity to SEQ ID NO: 41.

21. The tolerogenic compound of claim 19, wherein the antigen comprises SEQ ID NO: 41.

22. (canceled)

23. (canceled)

24. (canceled)

25. (canceled)

26. (canceled)

27. The tolerogenic compound of claim 13, wherein the antigen comprises an antigen associated with an autoimmune disease.

28. (canceled)

29. (canceled)

30. The tolerogenic compound of claim 29, wherein the autoimmune disease is multiple sclerosis and wherein the antigen comprises a sequence having at least 80%, 85%, 90%, 95%, 96%, 97%, 98%, 99%, or 100% sequence identity to any one of SEQ ID NO: 100, 71-99, 101-106 or 157-159, or a fragment thereof.

31. (canceled)

32. The tolerogenic compound of claim 31, wherein the autoimmune disease is type 1 diabetes and wherein the antigen comprises a sequence having at least 80%, 85%, 90%, 95%, 96%, 97%, 98%, 99%, or 100% sequence identity to any one of SEQ ID NO: 55-70 or 153-156, or a fragment thereof.

33-95. (canceled)
